(19) United States
(12) Patent Application Publication
(10) Pub. No.: US 2025/0270587 A1
(43) Pub. Date: Aug. 28, 2025

(54) Vector

(71) Applicants: Ospedale San Raffaele S.r.l., Milan (IT); Fondazione Telethon, Rome (IT)

(72) Inventors: Thomas Kerzel, Milan (IT); Mario Leonardo Squadrito, Milan (IT); Luigi Naldini, Milan (IT)

(21) Appl. No.: 18/039,734

(22) PCT Filed: Dec. 3, 2021
(86) PCT No.: PCT/EP2021/084276

§ 371 Date: Jun. 1, 2023

(30) Foreign Application Priority Data
Dec. 3, 2020 (GB) 2019108.6

(51) Int. Cl. C12N 15/86; A61K 38/21; A61K 48/00; A61P 35/00; C07K 14/56

(52) U.S. Cl. CPC C12N 15/86; A61K 38/212; A61K 48/0058; A61P 35/00; C07K 14/56; C12N 2740/15043; C12N 2830/008

(57) ABSTRACT

A vector for liver and/or splenic phagocyte-specific expression, wherein the vector comprises a transgene operably linked to one or more expression control sequence.

### CROSS-REFERENCE TO RELATED APPLICATIONS

This application is a U.S. National Phase of International Patent Application No. PCT/EP2021/084276, filed on Dec. 3, 2021, which claims priority from Application No. 2019108.6, filed on Dec. 3, 2020, in the United Kingdom.

### INCORPORATION BY REFERENCE FOR MATERIAL SUBMITTED ELECTRONICALLY

Incorporated by reference in its entirety is a computer-readable nucleotide/amino acid sequence listing identified as follows: “59163_Seqlisting.txt”, which was created on May 30, 2023, and is 72,004 bytes in size.

### FIELD OF THE INVENTION

The present invention relates to vectors for phagocyte-specific expression, particularly liver and/or splenic phagocyte-specific expression. The invention also relates to cells, pharmaceutical compositions and cancer vaccines comprising said vector and their use in therapy, including treatment or prevention of cancer, for example liver metastases.

### BACKGROUND TO THE INVENTION

The liver is involved in several biological functions, including detoxification, clearance of protein and cells, and metabolic functions among others (Robinson, M. W., et al., Cell Mol Immunol, 2016. 13(3): p. 267-76). In order to preserve the liver from immunological reactions that might damage it, the liver is characterized by an immunosuppressive environment that limits immune activation (Horst, A. K., et al., Cell Mol Immunol, 2016. 13(3): p. 277-92). Due to its immunosuppressive environment several tumour types are prone to spread towards the liver giving rise to liver metastases (Grakoui, A. and I. N. Crispe, Cell Mol Immunol, 2016. 13(3): p. 293-300; and Thomson, A. W. and P. A. Knolle, Nat Rev Immunol, 2010. 10(11): p. 753-66).

The liver is one of the most common sites for cancer metastasis, accounting for nearly 25% of all cases. A variety of primary tumors may be the source for metastasis, however, colorectal adenocarcinomas are the most common considering the overall number of patients affected (Griscom, J. T. and Wolf, P. S., 2020. Cancer, Liver Metastasis. In StatPearls. StatPearls Publishing). Liver metastases are linked to poor prognosis and often constitute the cause of death of cancer patients.

Surgical resection remains the gold standard for anatomically resectable liver metastases. Strategies to improve the chances of resection include neoadjuvant chemotherapy, portal vein embolization to increase the future liver remnant, or a two-stage resection versus a combined one-stage resection of the primary tumor, and hepatic lesions (Griscom, J. T. and Wolf, P. S., 2020. Cancer, Liver Metastasis. In StatPearls. StatPearls Publishing). However, the five-year survival after curative resection of hepatic lesions for patients with colorectal metastases has been reported as only 25% to 58% with a median survival length of 74 months (Pawlik, T. M., et al., 2005. Annals of surgery, 241(5), p. 715).

Thus, there is a demand for improved treatments for cancers such as liver metastases.

### SUMMARY OF THE INVENTION

The inventors have found that a vector (e.g. a lentiviral vector) driving transgene expression from a phagocyte-specific promoter (e.g. a M2-like macrophage-specific promoter such as the MRC1 promoter) can be used to drive selective transgene expression in Kupffer cells (KCs), and also in MRC1+ splenic macrophages and liver sinusoidal endothelial cells (LSECs).

The inventors have also found that miRNA target sequences (e.g. miRNA target sequences for miR-126-3p and miR-122-5p) can be used to increase the specificity of the transgene expression in the desired cells by abating transgene expression in other cells. For example, miRNA target sequences can be used to abate transgene expression in hepatocytes and/or LSECs.

The inventors have found that a vector driving transgene expression from a phagocyte-specific promoter in combination with miRNA target sequences can be used to selectively promote transgene expression in Kupffer cells, especially in the presence of experimental liver metastases (LMS), and to a lesser extent in splenic MRC1-positive macrophages.

The inventors have found that such vectors can be used to deliver molecules with anti-tumour activity (e.g. interferon-α, IFNα) to liver metastases. Mice treated with such a vector expressed robust and sustained levels of IFNα with no sign of hepatotoxicity, neutropenia or strong leukopenia. When the vector was administered to mice hosting experimental LMS, IFNα expression delayed tumour growth, reprogrammed tumour-associated macrophages (TAMs) and promoted adaptive immunity. The vector can be used to reduce the systemic toxic effects associated with non-selective transgene expression, by selectively delivering a therapeutic transgene to tumours (e.g. liver metastases).

The inventors have also found that such vectors can be used as tumour vaccines to promote adaptive immunity against tumour antigens (TAs) by driving expression of tumour antigens in antigen presenting cells (APCs). The inventors have found that a vector expressing a surrogate tumour antigen (e.g. chicken ovalbumin, OVA) strongly increased the number of cancer cell specific T cells indicating that the platform can be used to promote adaptive immunity against specific TAs. The vector may therefore be used as a cancer vaccine.

In one aspect, the present invention provides a vector for liver and/or splenic phagocyte-specific expression.

Suitably, the phagocytes targeted in the present invention are selected from one or more of: a macrophage, such as a M2-like macrophage and/or MRC1+ macrophage; a dendritic cell; and an endothelial cell, such as a liver sinusoidal endothelial cell.

Suitably, the phagocytes targeted in the present invention are selected from one or more of: a resident macrophage (e.g., a Kupffer cell); a liver sinusoidal endothelial cell; a splenic macrophage; a tumour-associated macrophage; and/or a monocyte-derived macrophage.

In some embodiments, the phagocytes targeted in the present invention are Kupffer cells.

The vector may comprise a transgene operably linked to one or more expression control sequence.

In another aspect, the invention provides a vector for liver and/or splenic phagocyte-specific expression, wherein the vector comprises a transgene operably linked to one or more expression control sequence.

In another aspect, the invention provides a vector for Kupffer cell-specific expression, wherein the vector comprises a transgene operably linked to one or more expression control sequence.

In another aspect, the invention provides a vector comprising a transgene operably linked to one or more liver and/or splenic phagocyte-specific expression control sequence.

In some embodiments, the one or more expression control sequence comprises:
(a) a phagocyte-specific promoter and/or enhancer; and/or
(b) one or more miRNA target sequence.

In some embodiments, the one or more expression control sequence comprises a phagocyte-specific promoter and/or enhancer. In some embodiments, the one or more expression control sequence comprises one or more miRNA target sequence. In some embodiments, the one or more expression control sequence comprises a phagocyte-specific promoter and/or enhancer, and one or more miRNA target sequence.

In some embodiments, the phagocyte-specific promoter and/or enhancer is a liver and/or splenic phagocyte-specific promoter and/or enhancer.

In some embodiments, the one or more miRNA target sequence suppresses expression in non-phagocyte (e.g. non-liver and/or splenic phagocyte) cells. In preferred embodiments, the one or more miRNA target sequence suppresses expression in non-liver phagocyte cells (i.e. in cells other than liver phagocyte cells).

In some embodiments, the phagocyte is a macrophage, optionally an M2-like macrophage and/or MRC1+ macrophage; dendritic cell; or liver sinusoidal endothelial cell.

In preferred embodiments, the phagocyte is a liver-resident phagocyte. In preferred embodiments, the phagocyte is a liver-resident macrophage. In preferred embodiments, the phagocyte is a Kupffer cell.

In some embodiments, vector comprises from 5′ to 3′: the phagocyte-specific promoter and/or enhancer—the transgene—the one or more miRNA target sequence.

In some embodiments, phagocyte-specific promoter and/or enhancer is selected from the group consisting of: a MRC1 promoter and/or enhancer; a TIE2 promoter; an ITGAM promoter and/or enhancer; a CD86 promoter and/or enhancer; a CD274 promoter and/or enhancer; a CD163 promoter and/or enhancer; a LYVE1 promoter and/or enhancer; a STAB1 promoter and/or enhancer; a ITGAX promoter and/or enhancer; a SIRPA promoter and/or enhancer; a TIE2 promoter and/or enhancer; a CHIL3 promoter and/or enhancer; a CD68 promoter and/or enhancer; a CSF1R promoter and/or enhancer; a VCAM1 promoter and/or enhancer; a PTGS1 promoter and/or enhancer; and a C1QA promoter and/or enhancer; a fragment thereof, or a combination thereof. In preferred embodiments, the phagocyte-specific promoter and/or enhancer is a MRC1 promoter and/or enhancer or a fragment thereof. In some embodiments, the phagocyte-specific promoter and/or enhancer comprises a nucleotide sequence having at least 70% identity to SEQ ID NO: 1, or a fragment thereof. Suitably, the phagocyte-specific promoter and/or enhancer is inducible.

The one or more miRNA target sequence may suppress expression in some liver and/or spleen cell populations.

In some embodiments, the one or more miRNA target sequence suppresses transgene expression in hepatocytes. In some embodiments, the one or more miRNA target sequence suppresses transgene expression in liver sinusoidal endothelial cells (LSECs). In some embodiments, the one or more miRNA target sequence suppresses transgene expression in splenic phagocytes. In some embodiments, the one or more miRNA target sequence suppresses transgene expression in splenic macrophages. In some embodiments, the one or more miRNA target sequence suppresses transgene expression in hepatocytes, liver sinusoidal endothelial cells (LSECs) and/or splenic phagocytes. In some embodiments, the one or more miRNA target sequence comprises: (a) one or more miR-126 target sequence; and/or (b) one or more miR-122 target sequence. In some embodiments, the one or more miRNA target sequence comprises four miR-126 target sequences and/or four miR-122 target sequences.

In some embodiments, the miR-126 target sequence comprises or consists of SEQ ID NO: 3.

In some embodiments, the miR-122 target sequence comprises or consists of SEQ ID NO: 4.

In a preferred embodiment, the vector comprises a transgene operably linked to (a) a MRC1 promoter and/or enhancer, or a fragment thereof; and (b) one or more miR-126 target sequence and/or one or more miR-122 target sequence.

In some embodiments, the transgene encodes a therapeutic polypeptide. In some embodiments, the transgene encodes an antigenic polypeptide.

In some embodiments, the transgene encodes a cytokine, optionally wherein the cytokine is interferon-alpha, interferon-beta, interferon-gamma, IL2, IL2, TNF-alpha, CXCL9 or IL1-beta. In some embodiments the transgene encodes interferon-alpha. In some embodiments, the transgene encodes an amino acid sequence having at least 70% identity to SEQ ID NO: 8. In some embodiments, the cytokine is IL10, IL15 or IL18. In some embodiments, the cytokine is IL10. In some embodiments, the transgene encodes an amino acid sequence having at least 70% identity to SEQ ID NO: 38. In some embodiments, the cytokine is IL12. In some embodiments, the transgene encodes an amino acid sequence having at least 70% identity to SEQ ID NO: 37 or 46.

In some embodiments, the transgene encodes a tumour-associated antigen, optionally wherein the tumour-associated antigen is carcinoembryonic antigen (CEA), melanoma associated antigen (MAGE) family, cancer germline (CAGE) family, B melanoma antigen (BAGE-1), synovial sarcoma x breakpoint 20 (SSX-2), Sarcoma antigen (SAGE) family, LAGE1, NY-ESO-1, HER2, EGFR, MUC-1 or GAST.

In some embodiments, the transgene is further operably linked to one or more regulatory elements. In some embodiments, the transgene is further operably linked to a Woodchuck Hepatitis Virus Posttranscriptional Regulatory Element (WPRE). In some embodiments, the transgene is further operably linked to a destabilising domain (e.g. a dihydrofolate reductase destabilising domain).

In some embodiments, the vector is a viral vector. For example, the vector may be a lentiviral vector, a retroviral vector, an adenoviral vector, an adeno-associated viral vector, or a herpes simplex viral vector. In some embodiments the vector is a lentiviral vector. In some embodiments, the vector is an integrating viral vector. In some embodiments, the vector is a non-integrating viral vector. For example, the vector may be integrase proficient or integrase deficient.

In some embodiments, the vector is a viral vector particle. In some embodiments, the viral vector particle is VSV-G pseudotyped. In some embodiments, the vector is a VSV-G pseudotyped lentiviral vector. In some embodiments the viral vector particle is substantially devoid of surface-exposed CD47 and/or HLA molecules. Suitably, the viral vector is produced in a viral particle producer or packaging cell which has been genetically engineered to decrease expression of CD47 and/or HLA molecules on the surface of the cell.

The vector may specifically express the transgene in phagocytes. In some embodiments:
(i) expression of the transgene in phagocytes transduced by the vector is greater than expression of the transgene in other cells transduced by the vector; and/or
(ii) the transgene is substantially not expressed in cells other than the phagocytes, when transduced by the vector; and/or
(iii) the transgene is substantially not expressed in lung cells, bone marrow cells and/or blood cells, when transduced by the vector; and/or
(iv) the transgene is substantially only expressed in some liver cells and/or some splenic cells; and/or
(v) expression of the transgene in Kupffer cells is at least ten times greater than expression in hepatocytes, when transduced by the vector; and/or
(vi) the transgene is substantially not expressed in hepatocytes when transduced by the vector.

In some embodiments, expression of the transgene in phagocytes transduced by the vector is greater than expression of the transgene in other cells transduced by the vector. In some embodiments, the transgene is substantially not expressed in cells other than the phagocytes, when transduced by the vector. In some embodiments, the transgene is substantially not expressed in lung cells, bone marrow cells and/or blood cells, when transduced by the vector. In some embodiments, expression of the transgene in Kupffer cells is at least ten times greater than expression in hepatocytes, when transduced by the vector. In some embodiments, the transgene is substantially not expressed in hepatocytes when transduced by the vector.

In some embodiments, the transgene is substantially only expressed in liver cells and/or splenic cells, and optionally substantially not expressed in hepatocytes when transduced by the vector.

In another aspect, the present invention provides a cell comprising the vector of the invention.

In another aspect, the present invention provides a pharmaceutical composition comprising the vector or cell of the invention.

In another aspect, the present invention provides a cancer vaccine comprising the vector or cell of the invention.

In another aspect, the present invention provides a method of making the viral vector particle of the invention, comprising introducing the vector of invention into a viral particle producer or packaging cell.

In another aspect, the present invention provides use of a vector of the invention for transducing or transfecting a cell. The use may, for example, be an in vitro or in vivo use.

In another aspect, the present invention provides a method of making the cell of the invention, comprising introducing the vector of invention into a cell. The method may, for example, be an in vitro or in vivo method.

In another aspect, the present invention provides the vector, cell or pharmaceutical composition of the invention for use in therapy. In another aspect, the present invention provides the vector, cell or pharmaceutical composition of the invention for use in the treatment or prevention of cancer.

In another aspect, the present invention provides a method of treating a disease comprising introducing the vector, cell or pharmaceutical composition of the invention into a cell. The method may, for example, be an ex vivo or in vivo method.

In another aspect, the present invention provides a method of treating or preventing cancer comprising administering the vector, cell or pharmaceutical composition of the invention to a subject in need thereof.

In some embodiments, the vector, cell or pharmaceutical composition is administered by intravenous injection, intraportal injection or intrahepatic artery injection.

### DESCRIPTION OF DRAWINGS

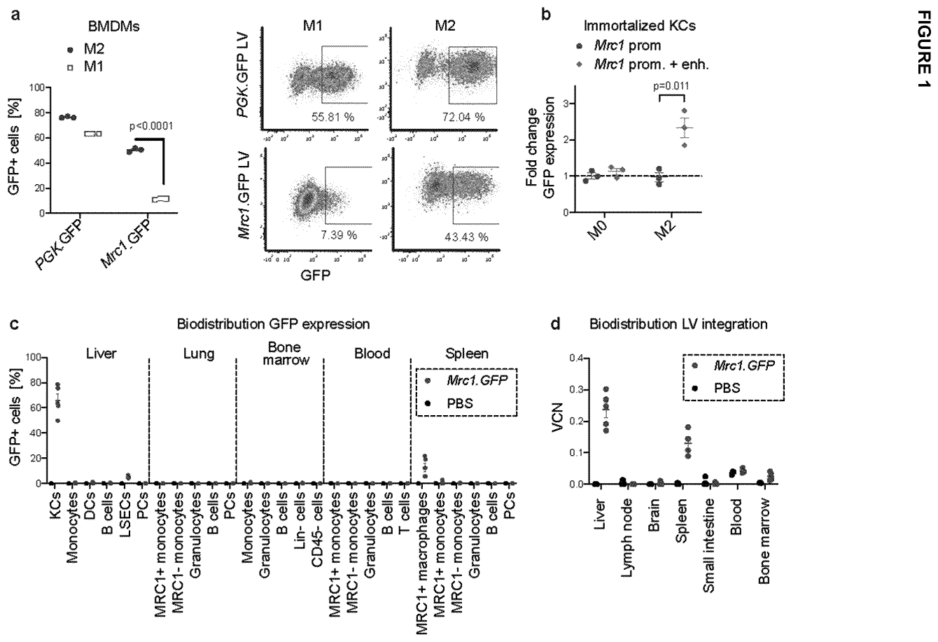

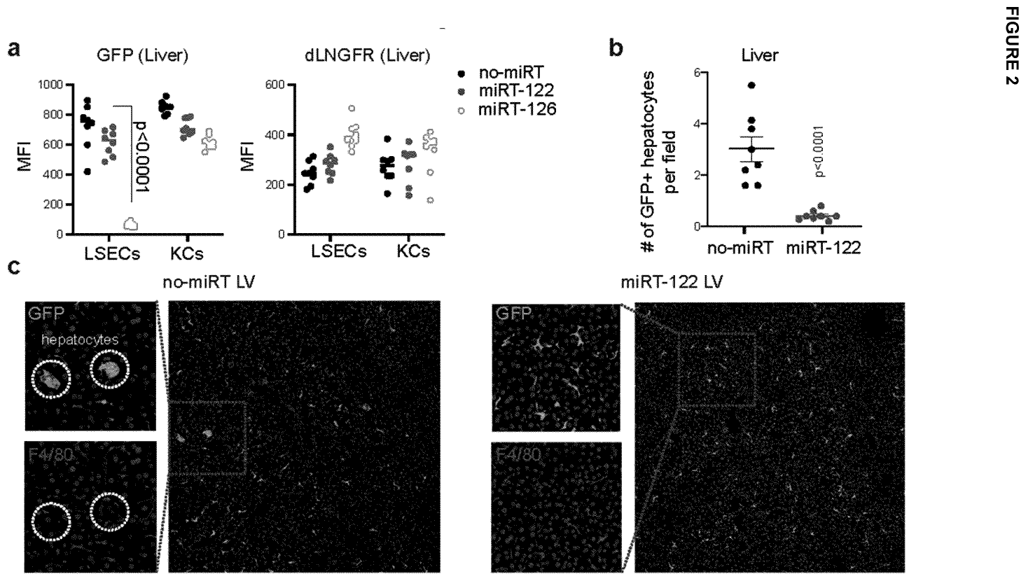

FIG. 1
(a) Flow cytometry analysis of BMDMs previously transduced with the indicated LVs and polarized with LPS+IFNγ(M1) or IL4 (M2). Left, GFP percentage (n=3 biological replicates, unpaired t test). Right, representative FACS plots of GFP expression for PGK.GFP (top) or Mrc1.GFP (bottom) transduced BMDMs gated on all viable cells.
(b) Mouse enhancer 6 (SEQ ID NO: 27) was inserted upstream to a vector expressing GFP from the Mrc1 promoter. Immortalized Kupffer cells (iKCs) were then transduced with the resulting LV and polarized with 50 ng of IL4 for 7 days. Panel shows fold change of GFP expression (median fluorescence intensity, MFI) in iKCs transduced with the enhancer containing construct (Mrc1 prom.+enh.) vs. promoter only construct (Mrc1 prom) after 7 days of IL4 stimulation (M2) or unstimulated (M0, n=3 independent experiments, unpaired t test).
(c) Flow cytometry analysis showing GFP expression of the indicated cell types in liver, lung, bone marrow, blood and spleen from mice treated with either the Mrc1.GFP LV or PBS (n=5 mice/group).
(d) VCN analysis by digital droplet PCR of different organs from mice treated with the Mrc1.GFP LV or PBS (n=4-5 mice/group from 2 independent experiments).

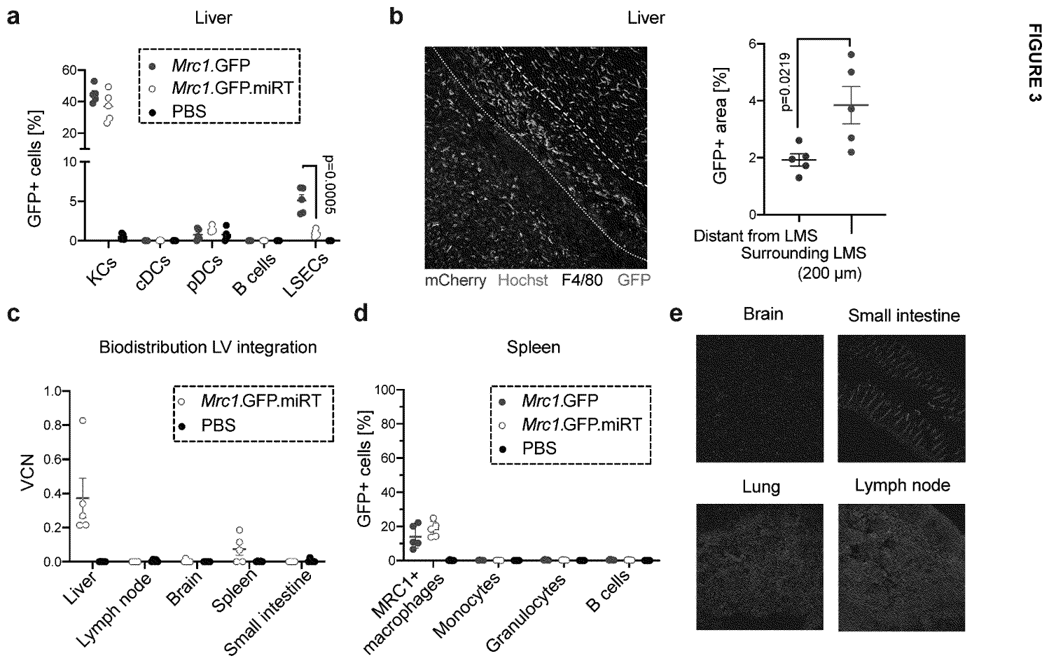

FIG. 2
(a) Mean fluorescence intensity (MFI) of GFP (left) and LNGFR (right) in LSECs and KCs measured by flow cytometry (n=8 mice/group, unpaired t test)
(b) Number of GFP expressing hepatocytes per frame identified by immunofluorescent analysis of liver sections. Per mice, the mean count of 5-6 images taken with a 10× magnification was measured (n=8 mice/group, Unpaired t test)
(c) Representative confocal images of liver sections from mice injected with the no-miRT (left) or the miRT-122 LV (right) stained for GFP (green), F4/80 (red) and nuclei (blue). Images were taken with a 10× magnification. Additional magnified images, in the area indicated by the dashed lines, show GFP staining (upper part) or F4/80 staining (lower part) in combination with nucleus staining.

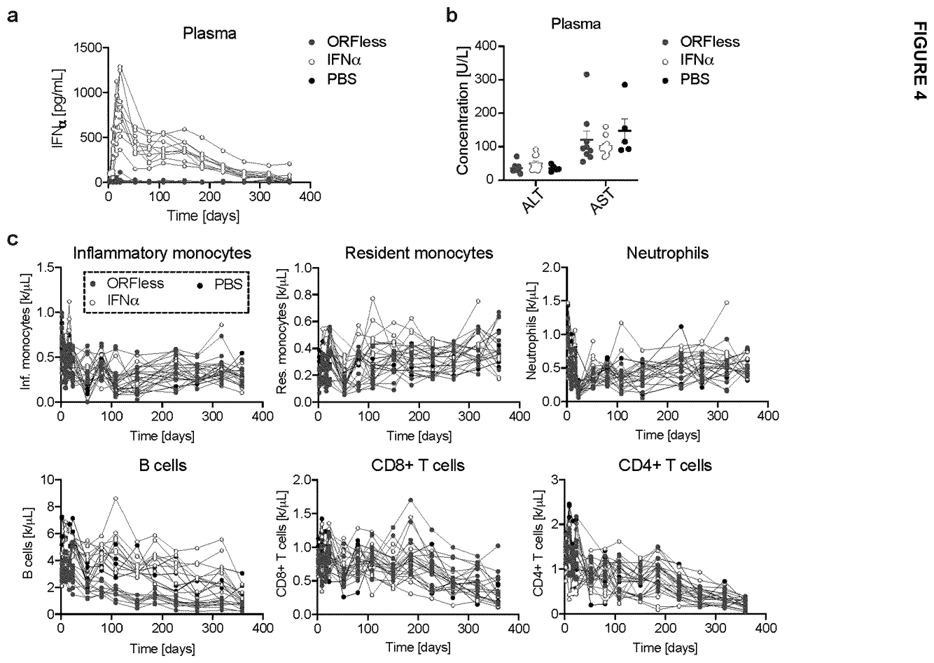

FIG. 3
(a) Flow cytometry analysis of the liver of mice treated with either the Mrc1.GFP LV, the Mrc1.GFP.miRT LV or PBS (n=5 mice/group, unpaired t test).
(b) Confocal imaging analysis of GFP expression of in the border area of mCherry MC38 liver metastases from Mrc1.GFP.miRT LV-treated mice. Liver sections were stained for GFP (green), mCherry (red), F4/80 (grey) and nuclei (blue). The border area was defined as the tumor surrounding area up to a distance of 200 μm. Pictures were taken with a 10× magnification. A representative image is shown on the left indicating the interface between tumor and liver tissue with a dotted line and the distance of 200 μm to the liver metastasis is marked by a dashed line. Right, quantification of the GFP area in percent in the tumor boarder area and the area distant to the tumor (n=5 mice/group, unpaired t test).
(c) VCN analysis by digital droplet PCR of different organs from mice treated with the indicated LV or PBS (n=5 mice per group).
(d) Flow cytometry analysis of spleens of mice treated with the indicated LVs showing percentage of distinct types of cells expressing GFP (n=5 mice per group).
(e) Representative immunofluorescent analysis of sections of the indicated organs stained for GFP (green) and nuclei (blue) taken with a 10× magnification (n=5).

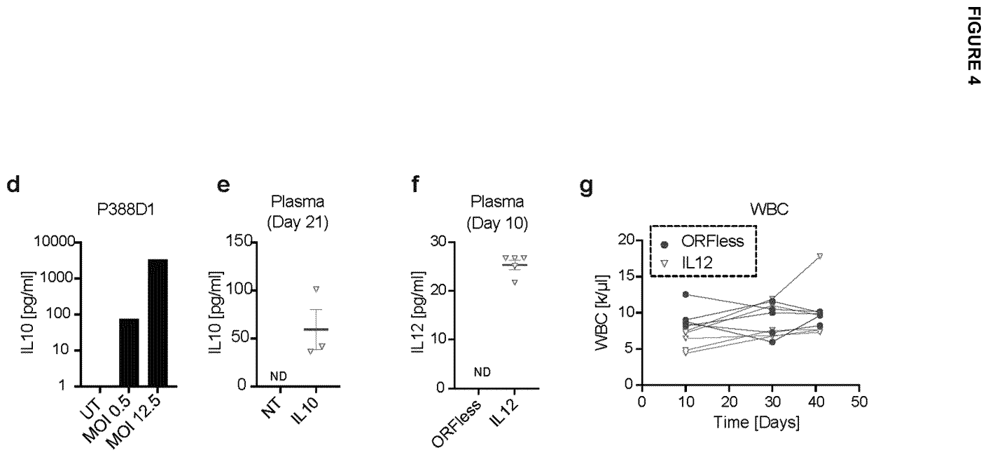

FIG. 4
(a) ELISA analysis showing IFNα concentration in the plasma of mice treated with the Mrc1.IFNα.miRT (IFNα), the Mrc1.ORFless.miRT (ORFless) LV or PBS over a period of 1 year (n=5-10 mice/group).
(b) Concentration of the indicated hepatic enzymes in the plasma of mice treated with the Mrc1.IFNα.miRT (IFNα), the Mrc1.ORFless.miRT (ORFless) LV or PBS (n=5-10 mice-group).
(c) Hemacytometer-normalised flow cytometry analysis of blood from mice treated with the Mrc1.IFNα.miRT (IFNα), the Mrc1.ORFless.miRT (ORFless) LV or PBS showing cell count of the indicated populations over a period of 1 year (n=5-10 mice-group).
(d) IL10 cDNA (SEQ ID NO: 39) was introduced at the place of the IFNα in the Mrc1.IFNα.miRT LV originating the Mrc1.IL10.miRT LV. The resulting LV was used to transduce the P388D1 monocytic cell line at distinct multiplicity of infection (MOI). IL10 concentration in the transduced P388D1-conditioned cell culture medium was measured by using ELISA.
(e) A single dose ranging from 1*10⁷ to 5*10⁷ TU per mouse of Mrc1.IL10.miRT (IL10) LV was delivered to 5 week old mice i.v. Plasma was collected after 21 days from treatment. Panel shows IL10 concentration in the plasma of untreated (NT) or Mrc1.IL10.miRT-treated mice by using ELISA (n=3 mice-group; ND indicates undetected levels in NT mice).
(f) The DNA encoding (SEQ ID NO: 40) for a 557 amino acid protein forming a single chain functional IL12 molecule, where: 1) amino acids from 1 to 23 are the signal peptide of mouse IL12 beta subunit, 2) amino acids from 24 to 28 are a linker sequence composed of the amino acidic sequence AGQLM (SEQ ID NO: 41), 3) amino acids from 29 to 340 are amino acids 23 to 335 of beta subunit of mouse IL12, 4) amino acids from 341 to 360 are a linker sequence composed of the amino acidic sequence RRAGGGGSGGGGSGGGGSRT (SEQ ID NO: 42), 5) amino acids from 361 to 553 are mouse IL12 subunit alpha (isoform 1) from amino acids 44 to 236, 6) amino acids from 554 to 557 are a termination sequence composed of the amino acidic sequence TRAS (SEQ ID NO: 43). Single chain IL12 cDNA was then inserted at the place of the IFNα in the Mrc1.IFNα.miRT LV originating the Mrc1.IL12.miRT LV. A single dose of 2*10⁶ TU per mouse of Mrc1.IL12.miRT LV was delivered to 6 week old mice i.v. Plasma was collected after 10 days from treatment. Panel shows IL12 concentration in the plasma of Mrc1.ORFless.miRT (ORFless) or Mrc1.IL12.miRT (IL12) LV-treated mice measured by using ELISA (n=5 mice-group; ND indicates undetected levels in Mrc1.ORFless.miRT-treated mice).
(g) Panel shows whole blood cell (WBC) count at the indicated time points from treatment with either Mrc1.ORFless.miRT LV (ORFless) or Mrc1.IL12.miRT LV (IL12) i.v. Of note, mice were challenged with MC38.OVA cancer cells subcutaneously at day 14 from LV treatment.

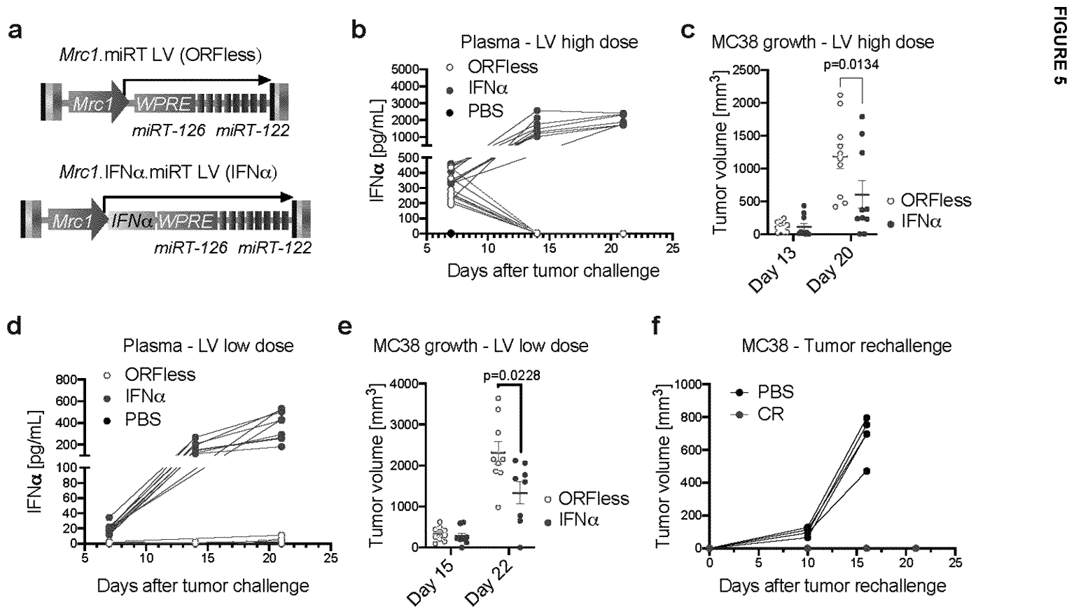

FIG. 5
(a) Schematics of the LVs used in therapeutic experiments.
(b) ELISA analysis showing IFNα concentration in the plasma of mice treated with Mrc1.IFNα.miRT (IFNα) or the Mrc1.ORFless.miRT (ORFless) LV at day 6 from tumor challenge at a dose of 3×10\hat{ }8 TU/mouse or PBS (n=5-10 mice/group).
(c) Magnetic resonance imaging (MRI) analysis showing the cumulative volume of liver metastases at the indicated time points from challenge with MC38 metastases in mice treated with the Mrc1.IFNα.miRT (IFNα) or the Mrc1.ORFless.miRT (ORFless) LV (n=10 mice/group, 2 way ANOVA with Sidak correction).
(d) ELISA analysis showing IFNα concentration in the plasma of mice treated with the Mrc1.IFNα.miRT (IFNα) or the Mrc1.ORFless.miRT (ORFless) LV at day 5 from tumor challenge at a dose of 3×10\hat{ }7 TU/mouse or PBS (n=5-10 mice/group).
(e) MRI analysis showing the cumulative volume of liver metastases at the indicated time points from challenge with MC38 metastases in mice treated with the Mrc1.IFNα.miRT (IFNα) or the Mrc1.ORFless.miRT (ORFless) LV (n=8, 9 mice/group, unpaired t test).
(f) Tumor growth of subcutaneous MC38 tumors in a complete responder (CR) or in PBS-treated mice (n=1, 5).

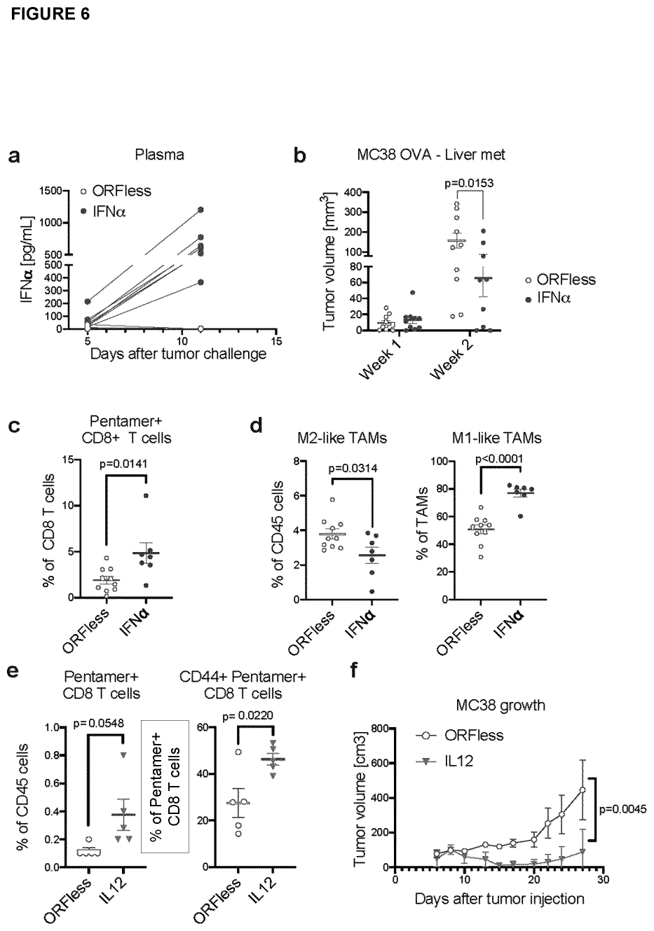

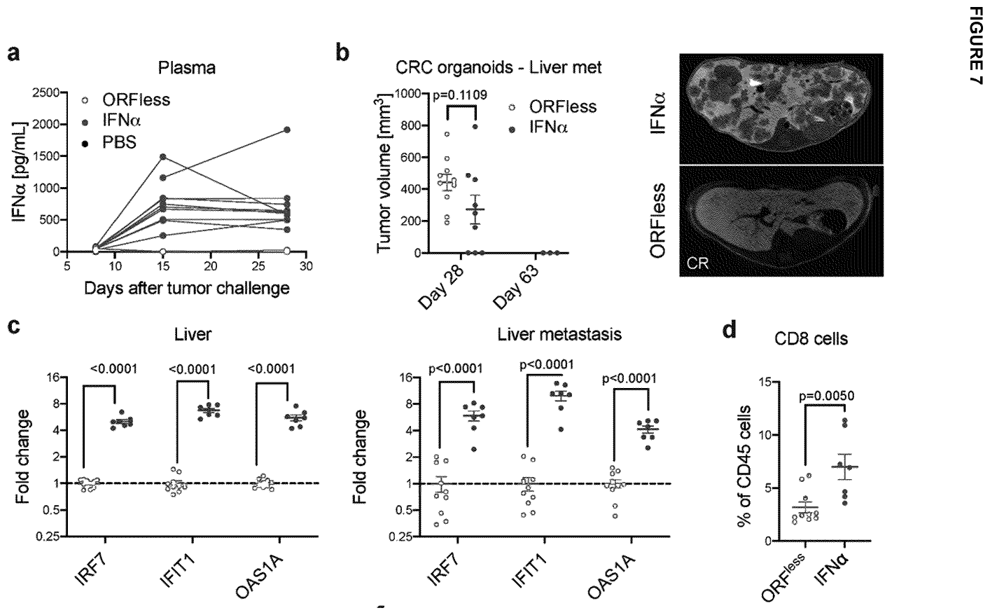

FIG. 6
(a) ELISA analysis showing IFNα concentration in the plasma of mice treated with Mrc1.IFNα.miRT (IFNα) or the Mrc1.ORFless.miRT (ORFless) LV at day 3 from tumor challenge (n=6-9 mice/group).
(b) MRI analysis showing the cumulative volume of liver metastases at the indicated time points from challenge with MC38 OVA metastases in mice treated with the Mrc1.IFNα.miRT (IFNα) or the Mrc1.ORFless.miRT (ORFless) LV (n=9, 10 mice/group, 2 way ANOVA with Sidak correction).
(c) Flow cytometry analysis of liver metastases from mice treated with the Mrc1.IFNα.miRT (IFNα) or the Mrc1.ORFless.miRT (ORFless) LV showing percentage of pentamer+(OVA specific CD8 T cells) out of total CD8+ T cells (n=7, 10 mice-group).
(d) Flow cytometry analysis of liver metastases from mice treated with the Mrc1.IFNα.miRT (IFNα) or the Mrc1.ORFless.miRT (ORFless) LV showing percentage of TAMs out of the indicated cell populations (n=7, 10 mice-group).
(e) Flow cytometry analysis of organs from mice that were implanted with subcutaneous MC38.OVA tumors 27 days before analysis. Mice were treated with the Mrc1.ORFless.miRT (ORFless) or the Mrc1.IL12.miRT (IL12) LV 14 days before tumor implantation. Left panel, percentage of liver pentamer+(OVA specific CD8 T cells) out of total CD45+ cells; right panel, percentage of CD44+ pentamer+CD8 T cells out of pentamer+CD8 T cells in the spleen (n=5 mice-group, unpaired t test).
(f) Tumor growth of MC38.OVA tumors implanted subcutaneously in mice that were treated with the Mrc1.ORFless.miRT (ORFless) or the Mrc1.IL12.miRT (IL12) LV 14 days before tumor implantation (n=5 mice-group, unpaired t test).

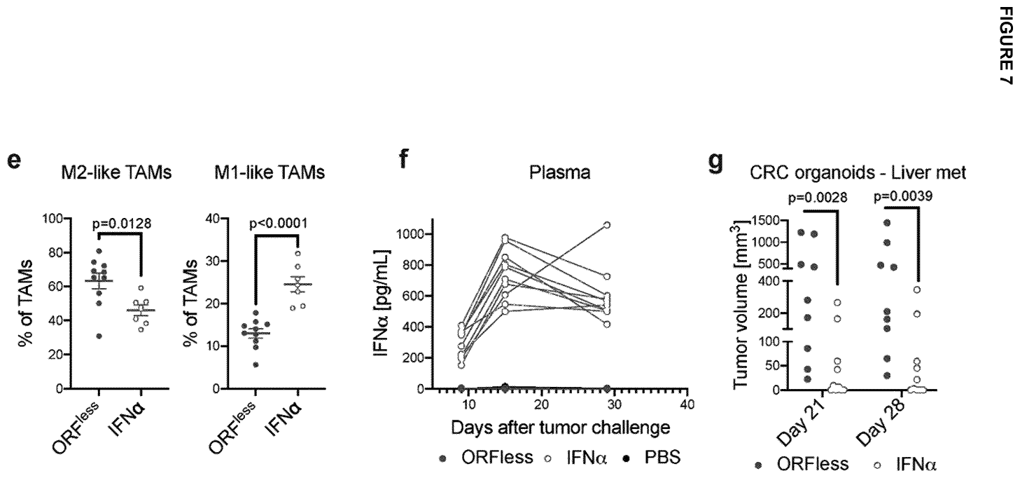

FIG. 7
(a) ELISA analysis showing IFNα concentration in the plasma of mice treated with Mrc1.IFNα.miRT (IFNα), the Mrc1.ORFless.miRT (ORFless) LV or PBS at day 7 from tumor challenge (n=5-10 mice/group).
(b) MRI analysis showing the cumulative volume of liver metastases at the indicated time points from challenge with CRC organoids originating metastases in mice treated with the Mrc1.IFNα.miRT (IFNα) or the Mrc1.ORFless.miRT (ORFless) LV (n=9, 10 mice/group, unpaired t test). Top right, representative MRI images. Arrows indicate single metastasis. Bottom right, a complete responder (CR) mouse from the Mrc1.IFNα.miRT (IFNα)-treated group.
(c) Gene expression analysis by digital droplet PCR of bulk liver and liver metastases showing, for the indicated genes, fold change in Mrc1.IFNα.miRT (IFNα) vs Mrc1.ORFless.miRT (ORFless, n=7, 10 mice/group, unpaired t test).
(d) Flow cytometry analysis of liver metastases from mice treated with the Mrc1.IFNα.miRT (IFNα) or the Mrc1.ORFless.miRT (ORFless) LV showing percentage of CD8+ T cells out of total CD45 cells (n=7, 10 mice-group).
(e) Flow cytometry analysis of liver metastases from mice treated with the Mrc1.IFNα.miRT (IFNα) or the Mrc1.ORFless.miRT (ORFless) LV showing percentage TAMs out of the indicated cell populations (n=7, 10 mice-group).
(f) ELISA analysis showing IFNα concentration in the plasma of mice treated with Mrc1.IFNα.miRT (IFNα), the Mrc1.ORFless.miRT (ORFless) LV or PBS at day 7 from tumor challenge (n=5-10 mice/group).
(g) MRI analysis showing the volume of cumulative liver metastases at the indicated time points from challenge with CRC organoids originating metastases in mice treated with the Mrc1.IFNα.miRT (IFNα) or the Mrc1.ORFless.miRT (ORFless) LV (n=9, 10 mice/group, unpaired t test).

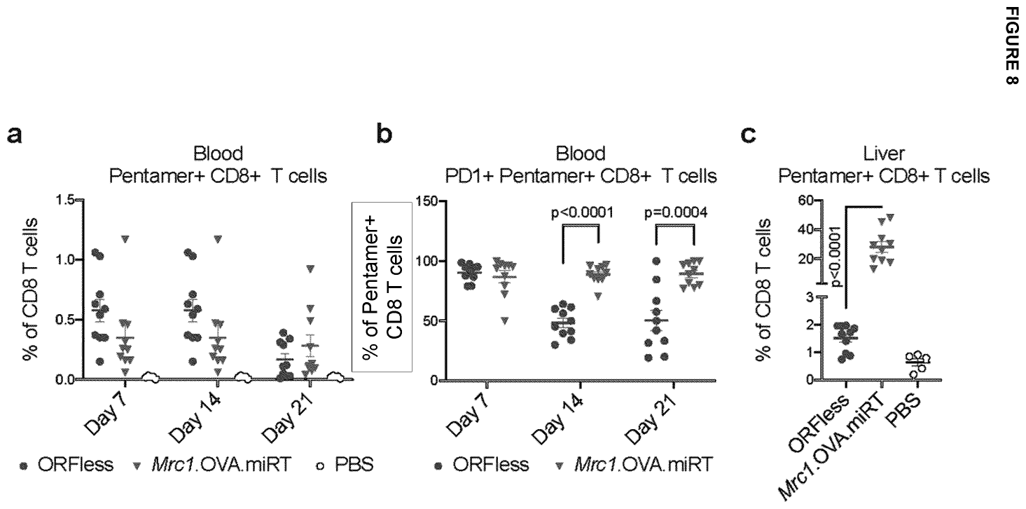

FIG. 8
(a) Flow cytometry analysis of blood from mice treated with the Mrc1.OVA.miRT, the Mrc1.ORFless.miRT (ORFless) LV or PBS showing percentage of pentamer+(OVA specific CD8 T cells) out of total CD8+ T cells (n=5, 10 mice-group).
(b) Flow cytometry analysis of blood from mice treated with the Mrc1.OVA.miRT or the Mrc1.ORFless.miRT (ORFless) LV showing percentage of PD1+ cells out of total pentamer+CD8+ T cells (n=5, 10 mice-group, unpaired t test).
(c) Flow cytometry analysis of liver from mice treated with the Mrc1.OVA.miRT, the Mrc1.ORFless.miRT (ORFless) LV or PBS showing percentage of pentamer+(OVA specific CD8 T cells) out of total CD8+ T cells (n=5, 10 mice-group, unpaired t test).

### DETAILED DESCRIPTION

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

### Phagocytes

The present invention relates to phagocyte-specific transgene expression, particularly liver and/or splenic phagocyte-specific transgene expression.

As used herein, a “phagocyte” is a specialised cell which is capable of phagocytosis. Phagocytosis may consist in recognition and ingestion of particles larger than 0.5 μm into a plasma membrane derived vesicle, known as phagosome. Phagocytes can ingest microbial pathogens and apoptotic cells. Thus, phagocytosis is essential not only for microbial elimination, but also for tissue homeostasis (Rosales, C. and Uribe-Querol, E., 2017. BioMed research international, 2017).

Suitably, the phagocytes targeted in the present invention are liver and/or splenic phagocytes.

As used herein, “liver phagocytes” may be phagocytes which are predominantly present in liver tissue and “splenic phagocytes” may be phagocytes which are predominantly present in spleen tissue.

Suitably, the phagocytes may be monocytes, macrophages, neutrophils, dendritic cells, eosinophils, fibroblasts, epithelial cells and/or endothelial cells.

Suitably, the phagocytes may be macrophages, dendritic cells and/or liver sinusoidal endothelial cells. For example, the phagocytes may be liver and/or splenic macrophages, liver and/or splenic dendritic cells, and/or liver sinusoidal endothelial cells.

Suitably, the phagocytes may be professional phagocytes (e.g. liver and/or splenic professional phagocytes), such as monocytes, macrophages, neutrophils, dendritic cells and eosinophils. In some embodiments, the phagocytes are macrophages and/or dendritic cells.

Suitably, the phagocytes may be non-professional phagocytes, such as fibroblasts, epithelial cells and/or endothelial cells. In some embodiments, the phagocytes are endothelial cells.

“Professional phagocytes” include monocytes, macrophages, neutrophils, dendritic cells, osteoclasts and eosinophils. These cells are in charge of eliminating microorganisms and of presenting them to cells of the adaptive immune system. In addition, fibroblasts, epithelial cells and endothelial cells can also perform phagocytosis. These “non-professional” phagocytes cannot ingest microorganisms but are important in eliminating apoptotic bodies (Rosales, C. and Uribe-Querol, E., 2017. BioMed research international, 2017).

### Macrophages

In some embodiments, the phagocytes are macrophages (e.g. liver and/or splenic macrophages).

Macrophages are innate immune cells that clear tissue from pathogens or other biological material. In adult mammals, macrophages are found in all tissues where they display great anatomical and functional diversity. In tissues, they are organized in defined patterns with each cell occupying its own territory. Macrophages have roles in almost every aspect of an organism's biology ranging from development, homeostasis, to repair through to immune responses to pathogens. In particular, tumours are abundantly populated by macrophages and they play an important role in tumour initiation, progression, and metastasis. (Ta, W., Chawla, A. and Pollard, J. W., 2013. Nature, 496, pp. 445-455).

Liver macrophages may include liver-resident macrophages, infiltrating macrophages (e.g. bone marrow (BM)-derived macrophages), avascular peritoneal macrophages, and splenic-derived monocytes. Splenic macrophages may include marginal zone macrophages (MZMΦs), marginal metallophilic macrophages (MMMΦs), and red pulp macrophages (RpMΦs).

In some embodiments, the phagocytes are M2-like macrophages and/or MRC1+ macrophages (e.g. liver and/or splenic M2-like and/or MRC1+ macrophages).

According to the activation state and functions of macrophages, they can be divided into M1-like (classically activated macrophage) and M2-like (alternatively activated macrophage). The M1 activation is induced by intracellular pathogens, bacterial cell wall components, lipoproteins, and cytokines such as interferon gamma and tumour necrosis factor alpha. M1-like macrophages are characterized with inflammatory cytokine secretion and production of nitric oxide (NO), resulting in an effective pathogen killing mechanism.

M2 activation is induced by fungal cells, parasites, immune complexes, complements, apoptotic cells, macrophage colony stimulating factor, IL-4, IL-13, IL-10, tumour growth factor beta. M2-like macrophages have high phagocytosis capacity, producing extracellular matrix (ECM) components, angiogenic and chemotactic factors, and IL-10. In addition to the pathogen defence, M2-like macrophages clear apoptotic cells, can mitigate inflammatory response, and promote wound healing. M2-like macrophages are commonly known as anti-inflammatory, pro-resolving, wound healing, tissue repair, and trophic or regulatory macrophages (Röszer, T., 2015. Mediators of inflammation, 2015).

M2-like macrophages may be identified based on the gene transcription or protein expression of a set of M2 markers as described in Röszer, T., 2015. Mediators of inflammation, 2015. These markers include transmembrane glycoproteins, scavenger receptors, enzymes, growth factors, hormones, cytokines, and cytokine receptors. Suitably, M2-like macrophages express one or more M2 macrophage markers such as MRC1 (CD206), CD163, CD209, Arginase-1, Chi313, FIZZ1, MGL-1, and Dectin-1.

In some embodiments, the phagocytes are MRC1+ macrophages.

Mannose receptor C-type 1 (MRC1) is also known as CD206, CLEC13D, and CLEC13DL. MRC1 is a C-type lectin primarily present on the surface of macrophages, immature dendritic cells and liver sinusoidal endothelial cells and mediates the endocytosis of glycoproteins. An example human MRC1 sequence is described under accession number UniProtKB P22897. An example mouse MRC1 sequence is described under accession number UniProtKB Q61830.

In mouse and humans, M2-like polarized macrophages, including tumour-associated macrophages (TAMs), or some resident macrophage populations such as Kupffer cells (KCs), some splenic macrophages, and adipose tissue macrophages express high levels of MRC1. MRC1 is also expressed by some dendritic cell (DC) populations and liver sinusoidal endothelial cells (LSECs) (Pandey, E., A. S. Nour, and E. N. Harris, Front Physiol, 2020. 11: p. 873).

In some embodiments, the phagocytes are resident macrophages (e.g. liver-resident macrophages or splenic-resident macrophages).

The majority of tissues in the body contain tissue-resident macrophage populations. Tissue-resident macrophages are known for their role as immune sentinels in the frontline of tissue defence where they are discretely positioned and transcriptionally programmed for the encounter with pathogens or environmental challenges (Davies, L. C., et al., 2013. Nature immunology, 14(10), p. 986).

Liver-resident macrophages (also called “liver macrophages”) include Kupffer cells and motile liver macrophages. Kupffer cells are maintained in the adult independently of the bone marrow and function to clear microorganisms and cell debris from the blood, and clear aged erythrocytes. Kupffer cell phenotypic markers may include F4/80^{hi}, CD11b^{lo}, CD169⁺, CD68⁺, Galectin-3⁺, and CD80^{lo/-}. Motile liver macrophages have an immune surveillance function and phenotypic markers may include F4/80⁺, CD11b⁺, and CD80^{hi} ((Davies, L. C., et al., 2013. Nature immunology, 14(10), p. 986).

Splenic-resident macrophages include marginal zone macrophages (MZMΦs), marginal metallophilic macrophages (MMMΦs), and red pulp macrophages (RpMΦs). Microanatomically, the spleen is divided into the white pulp and the red pulp (Rp), separated by the marginal zone (MZ). RpMΦs form a vast network inside the Rp and are characterized in mice by expression of F4/80^{high}CD68⁺CD11 b^{low/-} and intense autofluorescence. Inside the MZ, two populations of macrophages can be discerned. The MZMΦs typically express in their surface the C-type lectin SIGN-related 1 (SIGNR1) and a type I scavenger receptor called Macrophage Receptor with Collagenous structure (MARCO). MMMΦs are defined, among other molecules, by the expression of Sialic acid-binding Ig-like Lectin-1 (Siglec-1, Sialoadhesin, CD169) and MOMA-1.

In some embodiments, the phagocytes are infiltrating macrophages (e.g. liver-infiltrating macrophages or splenic-infiltrating macrophages), e.g. bone marrow (BM)-derived macrophages.

In some embodiments, the phagocytes are avascular peritoneal macrophages (PMs).

PMs reside in the peritoneal cavity with self-renewal abilities and exist as two distinct PM subsets i.e., large peritoneal macrophages (LPMs) and small peritoneal macrophages (SPMs). LPMs originate from embryonic precursors and represent the most abundant subset under steady conditions that display F4/80^{high}CD11 b^{high} MHCII^{low} phenotype. While SPMs are the minor subset with F4/80^{low} CD11b^{low} MHCII^{high} phenotype and originate from BM-derived myeloid precursors and predominantly appear during infection.

In some embodiments, phagocytes are monocyte-derived macrophages (e.g. liver and/or splenic monocyte-derived macrophages).

Monocytes circulate in the blood and are recruited to mucosal tissues or inflammation sites, where they can differentiate into monocyte-derived macrophages or monocyte-derived dendritic cells. MerTK, CD68, CD163, and the transcription factor MAFB are considered robust markers of macrophages, while dendritic cells express CD1a, CD1b, FcεRI, and CD226. Macrophages are large cells containing many phagocytic vesicles. By contrast, dendritic cells are smaller and display dendrites on their surface (Segura, E. and Coillard, A., 2019. Frontiers in immunology, 10, p. 1907).

In some embodiments, the phagocytes are tumour-associated macrophages (e.g. liver and/or splenic tumour-associated macrophages).

Tumour-associated macrophages (TAMs) are a class of macrophage present in high numbers in the microenvironment of solid tumours. Tumour-associated macrophages (TAMs) contribute to tumour progression at different levels: by promoting genetic instability, nurturing cancer stem cells, supporting metastasis, and taming protective adaptive immunity. TAMs can have a dual supportive and inhibitory influence on cancer, depending on the disease stage, the tissue involved, and the host microbiota (Mantovani, A., et al., 2017. Nature reviews Clinical oncology, 14(7), p. 399).

In some embodiments, the phagocytes are MRC1+ liver macrophages (e.g. Kupffer cells) and/or MRC1+ splenic macrophages.

In some embodiments, the phagocytes are Kupffer cells.

### Dendritic Cells

In some embodiments, the phagocytes are dendritic cells (e.g. liver and/or splenic dendritic cells).

Dendritic cells (DCs) are antigen-presenting cells of the mammalian immune system. Their main function is to process antigen material and present it on the cell surface to the T cells of the immune system.

In normal liver, DCs typically reside only around portal triads and, like DC in other peripheral sites, are able to efficiently capture, process, and transport antigens to regional lymphoid tissues. Compared to LSECs and KCs, freshly isolated hepatic DC are predominantly immature cells, expressing surface MHC but few costimulatory molecules necessary for T cell activation (Lau, A. H. and Thomson, A. W., 2003. Gut, 52(2), pp. 307-314).

Both conventional/myeloid DCs (cDC) and plasmacytoid DCs (pDC) at different maturation stages and different subsets are present in human spleen (Velásquez-Lopera, M. M., et al., 2008. Clinical & Experimental Immunology, 154(1), pp. 107-114).

### Endothelial Cells

In some embodiments, the phagocytes are endothelial cells (e.g. liver and/or splenic endothelial cells). For example, the phagocytes may be liver sinusoidal endothelial cells (LSECs).

LSECs have one of the highest endocytic capacities in the human body and can clear soluble macromolecules and small particles through endocytic receptors. Features used to identify LSECs include: (a) their high and rapid endocytic capacity, (b) fenestrae without diaphragm and organized in sieve plate, and (c) surface markers such as VEGFR3⁺CD34⁻ VEGFR2⁺ VE-Cadherin⁺ FactorVIII⁺ CD45⁻ or CD31⁺, LYVE-1⁺, L-SIGN⁺, Stabilin-1⁺, CD34⁻, PROX-1⁻ (Poisson, J., et al., 2017. Journal of hepatology, 66(1), pp. 212-227).

In some embodiments, the phagocytes are LSECs.

In another aspect, the invention provides a vector for LSEC-specific expression, wherein the vector comprises a transgene operably linked to one or more expression control sequence

### Vector

In one aspect, the present invention provides a vector for phagocyte-specific expression, particularly liver and/or splenic phagocyte-specific expression.

### Phagocyte-Specific Expression

The vector may be a phagocyte-specific expression vector, particularly a liver and/or splenic phagocyte-specific expression vector. The terms “phagocyte-specific expression”, “liver phagocyte-specific expression” and “splenic phagocyte-specific expression”, as used herein, may refer to the preferential or predominant expression of a transgene (e.g., as polypeptide or RNA) in the phagocytes as compared to other cells (e.g., blood, lung and bone marrow cells). In some embodiments, at least 50% of transgene expression occurs in the phagocytes. In some embodiments, at least 60%, 70%, 80%, 90% or 95% of transgene expression occurs in the phagocytes. In some embodiments, the transgene is substantially exclusively expressed in the phagocytes.

For example:
(i) expression of the transgene in phagocytes transduced by the vector may be greater than expression of the transgene in other cells transduced by the vector; and/or (ii) the transgene may be substantially not expressed in cells other than the phagocytes, when transduced by the vector; and/or
(iii) the transgene may be substantially not expressed in lung cells, bone marrow cells and/or blood cells, when transduced by the vector; and/or
(iv) the transgene may be substantially only expressed in some liver cells and/or some splenic cells; and/or
(v) expression of the transgene in Kupffer cells may be at least ten times greater than expression in hepatocytes, when transduced by the vector; and/or
(vi) the transgene may be substantially not expressed in hepatocytes when transduced by the vector.

Expression of the transgene may be determined by any suitable method known to the skilled person. For example, if the transgene is a reporter gene (e.g., GFP) flow cytometry analysis may be used to determine expression levels in different cell types. Alternatively, if the transgene is a reporter gene (e.g., GFP) immunofluorescent analysis (e.g., by confocal imaging analysis) may be used to determine expression levels in different cell types.

Suitably, expression of the transgene in phagocytes transduced by the vector may be greater than expression of the transgene in other cells transduced by the vector. For example, expression of the transgene in phagocytes transduced by the vector may be at least 10 times, at least 20 times, or at least 50 times, or at least 100 times greater than in other cells transduced by the vector.

Suitably, the transgene is substantially not expressed in cells other than the phagocytes, when transduced by the vector. For example, the percentage of the cells other than the phagocytes which express the transgene may be 5% or less, 2% or less, 1% or less, or 0%. For example, expression of the transgene in cells other than the phagocytes may be undetectable.

Suitably, the transgene is substantially not expressed in lung cells, bone marrow cells and/or blood cells, when transduced by the vector. For example, the percentage of lung cells, bone marrow cells and/or blood cells which express the transgene may be 5% or less, 2% or less, 1% or less, or 0%. For example, expression of the transgene in lung cells, bone marrow cells and/or blood cells may be undetectable.

Suitably, the transgene is substantially only expressed in liver cells and/or splenic cells. For example, the percentage of the cells types other than liver cells and/or splenic cells which express the transgene may be 5% or less, 2% or less, 1% or less, or 0%. For example, expression of the transgene in cell types other than liver cells and/or splenic cells may be undetectable.

Suitably, expression of the transgene in Kupffer cells may be at least ten times greater than expression in hepatocytes, when transduced by the vector. For example, expression of the transgene in Kupffer cells may be at least ten times greater, at least twenty times greater, or at least fifty times greater than expression in hepatocytes.

Suitably, the transgene may be substantially not expressed in hepatocytes when transduced by the vector. For example, the percentage of hepatocytes which express the transgene may be 5% or less, 2% or less, 1% or less, or 0%. For example, expression of the transgene in hepatocytes may be undetectable.

Suitably, expression of the transgene in Kupffer cells may be at least ten times greater than expression in LSECs, when transduced by the vector. For example, expression of the transgene in Kupffer cells may be at least ten times greater, at least twenty times greater, or at least fifty times greater than expression in LSECs.

Suitably, the transgene may be substantially not expressed in LSECs when transduced by the vector. For example, the percentage of LSECs which express the transgene may be 5% or less, 2% or less, 1% or less, or 0%. For example, expression of the transgene in LSECs may be undetectable.

If the vector is an integrating vector (e.g., integrase proficient) then copies of the vector may be, for example, specifically integrated into phagocytes, particularly liver and/or splenic phagocytes. For example:
(i) integration of the vector in liver and spleen may be greater than integration of the vector in other organs (e.g., lymph node, brain, small intestine, blood, bone marrow); and/or
(ii) integration of the vector may substantially occur in liver, spleen, optionally blood and optionally bone marrow; and/or
(iii) integration of the vector may substantially not occur in lymph node, brain, small intestine.

Integration of the vector may be determined by any suitable method known to the skilled person. For example, viral copy number analysis, e.g., by quantitative digital droplet PCR of different organs.

Suitably, integration of the vector in liver and spleen is greater than integration of the vector in other organs, such as lymph node, brain, small intestine, blood, bone marrow. For example, the viral copy number of liver and spleen may be at least 10 times, at least 20 times, or at least 50 times, or at least 100 times greater than in other organs.

Suitably, integration of the vector substantially occurs in liver, spleen, optionally blood and optionally bone marrow. For example, integration of the vector in the liver and spleen, optionally blood and optionally bone marrow, may be at least detectable.

Suitably, integration of the vector substantially does not occur in lymph node, brain, small intestine. For example, integration of the vector in in lymph node, brain, small intestine may be undetectable. All these biological compartments host resident macrophage populations that could potentially express the transgene upon systemic delivery of the vector.

### Viral Vector

Suitably, the vector of the present invention is a viral vector. The vector of the invention may be a lentiviral vector, although it is contemplated that other viral vectors may be used.

Other suitable viral vectors include those described in Lundstrom, K., 2018. Diseases, 6(2), p. 42. For example, other suitable viral vectors include a retroviral vector, an adenoviral vector, an adeno-associated viral vector, a herpes simplex viral vector, an alphaviral vector, a flaviviral vector, a rhabdoviral vector, a measles viral vector, a Newcastle disease viral vector, a poxviral vector, and a picornaviral vector.

The vector of the present invention may be in the form of a viral vector particle. Suitably, the viral vector of the present invention is in the form of a lentiviral vector particle.

The vector may be an integrating viral vector or a non-integrating viral vector. An “integrating viral vector” is capable of integrating into the host cell genome following transduction into the host cell. A “non-integrating viral vector” is not capable of integrating into the host cell genome following transduction into the host cell or demonstrates very weak integration capability.

Methods of preparing and modifying viral vectors and viral vector particles, such as lentiviral vectors, are well known in the art. Suitable methods are described in Merten, O. W., et al., 2016. Molecular Therapy-Methods & Clinical Development, 3, p. 16017; Nadeau, I. and Kamen, A., 2003. Biotechnology advances, 20(7-8), pp. 475-489; Ayuso, E., et al., 2010. Current gene therapy, 10(6), pp. 423-436; and Goins, W. F., et al., 2008. Methods Mol Biol. 433, pp. 97-113.

### Retroviral and Lentiviral Vectors

The vector of the present invention may be a retroviral vector or a lentiviral vector. The vector of the present invention may be a retroviral vector particle or a lentiviral vector particle.

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV).

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells. In contrast, other retroviruses, such as MLV, are unable to infect non-dividing or slowly dividing cells such as those that make up, for example, muscle, brain, lung and liver tissue.

A “lentiviral vector”, as used herein, is a vector which comprises at least one component part derivable from a lentivirus. Suitably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Optionally, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Optionally, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Optionally, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integrase-defective (i.e., integrase-deficient). Integration defective lentiviral vectors can be produced, for example, either by packaging the vector with catalytically inactive integrase (such as an HIV integrase bearing the D64V mutation in the catalytic site) or by modifying or deleting essential att sequences from the vector LTR, or by a combination of the above.

In some embodiments, the vector is an integrase-defective lentiviral vector. In some embodiments, the vector is an integrase-proficient lentiviral vector.

Vesicular stomatitis virus glycoprotein (VSV-G)-pseudotyped lentiviral vectors (LV) upon systemic delivery may efficiently and specifically target the liver and are preferentially internalized by liver and splenic phagocyte populations, although other cell types including endothelial cells and, hepatocytes, are also transduced (Milani, M., et al., Sci Transl Med, 2019. 11(493)). Thus, VSV-G-pseudotyped LVs constitute excellent tools to deliver genes of interest to the liver cell populations.

Suitably, the vector is VSV-G-pseudotyped. In some embodiments, the vector is a VSV-G-pseudotyped lentiviral vector particle.

Gene transfer into professional phagocytes and antigen presenting cells (APCs) is constrained by the presence of the CD47 molecules on LV particles. CD47-free LV show preserved infectivity and substantially increased susceptibility to phagocytosis. CD47-free LV more efficiently transduce professional phagocytes both ex vivo and in vivo, and induce a substantially higher rise in cytokine response upon systemic administration to mice, compared to CD47-bearing LV. CD47-free LV allow increased gene transfer efficiency into human primary monocytes, and have increased susceptibility to phagocytosis both ex vivo by primary human macrophages and in vivo when administered systemically to mice, compared to previously available LV. For example, VSV-G-pseudotyped LVs lacking CD47 molecules on their surface are even more efficiently uptaken by professional phagocytes of liver and spleen than CD47-bearing VSV-G-pseudotyped LVs.

An allogeneic human leukocyte antigen (HLA) e.g. MHC-I may also be recognised by the immune system. For example, antibodies may bind HLA epitopes directly. As a result, cells and enveloped viruses that comprise HLA proteins originating from an allogeneic source may be targeted and neutralised by the immune system. A decreased number or lack of surface-exposed HLA molecules is advantageous in viruses for use as vaccines, as the viruses will be less likely to be neutralised by antibodies binding to HLA.

Suitable methods of producing CD47-free and/or HLA-free vectors are described in WO 2019/219836.

In some embodiments, the vector is substantially devoid of surface-exposed CD47 and/or HLA molecules. In some embodiments the vector is a VSV-G-pseudotyped lentiviral vector particle substantially devoid of surface-exposed CD47 and/or HLA molecules.

The term “substantially devoid” as used herein means that there is a substantial decrease in the number of molecules that are expressed on the surface, in comparison to the number of molecules that are expressed on the surface of a vector produced in cells which have not been genetically engineered to reduce expression of the molecule (but under otherwise substantially identical conditions), such that the vectors exhibit a therapeutically useful increase in ability to transduce macrophages, phagocytes, antigen-presenting cells and/or monocytes, and/or induce a cytokine response upon systemic administration.

In some embodiments, the vector does not comprise any surface-exposed CD47 molecules and/or HLA molecules. In some embodiments, the vector is a VSV-G-pseudotyped lentiviral vector particle which does not comprise any surface-exposed CD47 molecules and/or HLA molecules.

### Adenoviral Vector

The vector of the present invention may be an adenoviral vector. The vector of the present invention may be an adenoviral vector particle.

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹².

Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Adeno-Associated Viral Vector

The vector of the present invention may be an adeno-associated viral (AAV) vector. The vector of the present invention may be in the form of an AAV vector particle.

The AAV vector or AAV vector particle may comprise an AAV genome or a fragment or derivative thereof. An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

AAVs occurring in nature may be classified according to various biological systems. The AAV genome may be from any naturally derived serotype, isolate or clade of AAV.

AAV may be referred to in terms of their serotype. A serotype corresponds to a variant subspecies of AAV which, owing to its profile of expression of capsid surface antigens, has a distinctive reactivity which can be used to distinguish it from other variant subspecies. Typically, an AAV vector particle having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. ITRs may be the only sequences required in cis next to the therapeutic gene. Suitably, one or more ITR sequences flank the transgene.

The AAV genome may also comprise packaging genes, such as rep and/or cap genes which encode packaging functions for an AAV particle. A promoter may be operably linked to each of the packaging genes. Specific examples of such promoters include the p5, p19 and p40 promoters. For example, the p5 and p19 promoters are generally used to express the rep gene, while the p40 promoter is generally used to express the cap gene. The rep gene encodes one or more of the proteins Rep78, Rep68, Rep52 and Rep40 or variants thereof. The cap gene encodes one or more capsid proteins such as VP1, VP2 and VP3 or variants thereof.

The AAV genome may be the full genome of a naturally occurring AAV. For example, a vector comprising a full AAV genome may be used to prepare an AAV vector or vector particle.

Suitably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This may reduce the risk of recombination of the vector with wild-type virus, and avoid triggering a cellular immune response by the presence of viral gene proteins in the target cell.

Typically, a derivative will include at least one inverted terminal repeat sequence (ITR), optionally more than one ITR, such as two ITRs or more. One or more of the ITRs may be derived from AAV genomes having different serotypes, or may be a chimeric or mutant ITR. A suitable mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The AAV genome may comprise one or more ITR sequences from any naturally derived serotype, isolate or clade of AAV or a variant thereof. The AAV genome may comprise at least one, such as two, AAV1, AAV2, AAV3, AAV4, AAV5, AAV6, AAV7, AAV8, AAV9, AAV10, or AAV11 ITRs, or variants thereof.

The one or more ITRs may flank the transgene at either end. The inclusion of one or more ITRs is can aid concatamer formation of the AAV vector in the nucleus of a host cell, for example following the conversion of single-stranded vector DNA into double-stranded DNA by the action of host cell DNA polymerases. The formation of such episomal concatamers protects the AAV vector during the life of the host cell, thereby allowing for prolonged expression of the transgene in vivo.

Suitably, ITR elements will be the only sequences retained from the native AAV genome in the derivative. Suitably, a derivative may not include the rep and/or cap genes of the native genome and any other sequences of the native genome. This may reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the AAV vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector particle may be encapsidated by capsid proteins. Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). The AAV vector may be in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery and/or decreased immunogenicity (humoral or cellular) compared to an AAV vector comprising a naturally occurring AAV genome. Increased efficiency of gene delivery, for example, may be effected by improved receptor or co-receptor binding at the cell surface, improved internalisation, improved trafficking within the cell and into the nucleus, improved uncoating of the viral particle and improved conversion of a single-stranded genome to double-stranded form.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle.

The capsid protein may be an artificial or mutant capsid protein. The term “artificial capsid” as used herein means that the capsid particle comprises an amino acid sequence which does not occur in nature or which comprises an amino acid sequence which has been engineered (e.g. modified) from a naturally occurring capsid amino acid sequence. In other words the artificial capsid protein comprises a mutation or a variation in the amino acid sequence compared to the sequence of the parent capsid from which it is derived where the artificial capsid amino acid sequence and the parent capsid amino acid sequences are aligned.

### Herpes Simplex Viral Vector

The vector of the present invention may be a herpes simplex viral vector. The vector of the present invention may be a herpes simplex viral vector particle.

Herpes simplex virus (HSV) is a neurotropic DNA virus with favourable properties as a gene delivery vector. HSV is highly infectious, so HSV vectors are efficient vehicles for the delivery of exogenous genetic material to cells. Viral replication is readily disrupted by null mutations in immediate early genes that in vitro can be complemented in trans, enabling straightforward production of high-titre pure preparations of non-pathogenic vector. The genome is large (152 Kb) and many of the viral genes are dispensable for replication in vitro, allowing their replacement with large or multiple transgenes. Latent infection with wild-type virus results in episomal viral persistence in sensory neuronal nuclei for the duration of the host lifetime. The vectors are non-pathogenic, unable to reactivate and persist long-term. The latency active promoter complex can be exploited in vector design to achieve long-term stable transgene expression in the nervous system.

HSV vectors transduce a broad range of tissues because of the wide expression pattern of the cellular receptors recognized by the virus. Increasing understanding of the processes involved in cellular entry has allowed targeting the tropism of HSV vectors.

### Other Viral Vectors

Other suitable viral vectors include those described in Lundstrom, K., 2018. Diseases, 6(2), p. 42.

The vector of the present invention may be an alphaviral vector. The vector of the present invention may be an alphaviral vector particle. The vector of the present invention may be a flaviviral vector. The vector of the present invention may be a flaviviral vector particle.

Self-amplifying ssRNA viruses comprise of alphaviruses (e.g. Semliki Forest virus, Sindbis virus, Venezuelan equine encephalitis virus, and M1) and flaviviruses (e.g. Kunjin virus, West Nile virus, and Dengue virus) possessing a genome of positive polarity. Alphaviruses have been mainly applied in preclinical gene therapy studies for cancer treatment. Alphavirus vectors can be delivered in the form of naked RNA, layered plasmid DNA vectors and recombinant replication-deficient or -proficient particles.

The vector of the present invention may be a rhabdoviral vector. The vector of the present invention may be a rhabdoviral vector particle. The vector of the present invention may be a measles viral vector. The vector of the present invention may be a measles viral vector particle.

Rhabdoviruses (e.g. rabies and vesicular stomatitis virus) and measles viruses carry negative strand genomes. Among rhabdoviruses, recombinant vesicular stomatitis virus (VSV) has been applied for preclinical gene therapy studies. Measles viruses (e.g. MV-Edm) have found a number of gene therapy applications.

The vector of the present invention may be a Newcastle disease viral vector. The vector of the present invention may be a Newcastle disease viral vector particle.

The ssRNA paramyxovirus Newcastle disease virus (NDV) replicates specifically in tumour cells and has therefore been frequently applied for cancer gene therapy.

The vector of the present invention may be a poxviral vector. The vector of the present invention may be a poxviral vector particle.

The characteristic feature of poxviruses is their dsDNA genome, which can generously accommodate more than 30 kb of foreign DNA. Poxviruses have found several applications as gene therapy vectors. For instance, vaccinia virus vectors have demonstrated potential for treatment of cancer. Vaccinia virus is large enveloped poxvirus that has an approximately 190 kb linear, double-stranded DNA genome. Vaccinia virus can accommodate up to approximately 25 kb of foreign DNA, which also makes it useful for the delivery of large genes. A number of attenuated vaccinia virus strains are known in the art that are suitable for gene therapy applications, for example the MVA and NYVAC strains.

The vector of the present invention may be a picornaviral vector. The vector of the present invention may be a picornaviral vector particle.

Picornoviruses are non-enveloped ssRNA viruses. Coxsackieviruses belonging to Picornaviridae, have been applied as oncolytic vectors.

### Expression Control Sequences

The vector of the present invention may comprise one or more expression control sequence. Suitably, the transgene is operably linked to one or more expression control sequence.

As used herein an “expression control sequence” is any nucleotide sequence which controls expression of a transgene, e.g., to facilitate and/or increase expression in some cell types and/or decrease expression in other cell types.

The expression control sequence and the transgene may be in any suitable arrangement in the vector, providing that the expression control sequence is operably linked to the transgene. The term “operably linked”, as used herein, means that the parts (e.g., transgene and one or more expression control sequence) are linked together in a manner which enables both to carry out their function substantially unhindered.

The expression control sequence may be a phagocyte-specific expression control sequence, particularly a liver and/or splenic phagocyte-specific expression control sequence (e.g., such that the vector specifically expresses a transgene in phagocytes, particularly liver and/or splenic phagocytes). Expression control sequences include promoters, enhancers, and 5′ and 3′ untranslated regions (e.g., miRNA target sequences).

The one or more expression control sequence may comprise: (a) a phagocyte-specific promoter and/or enhancer; and/or (b) one or more miRNA target sequence.

In some embodiments, the one or more expression control sequence comprises a phagocyte-specific promoter and/or enhancer, and, optionally, one or more miRNA target sequence.

The vector may, for example, comprise from 5′ to 3′: a phagocyte-specific promoter and/or enhancer—a transgene—one or more miRNA target sequence.

### Mrc1-Derived Expression Control Sequences

Suitably, the vector of the present invention may comprise one or more MRC1-derived expression control sequence.

As used herein, a “MRC1-derived expression control sequence” is an expression control sequence which includes any of the regulatory features present in the MRC1 gene. An example human MRC1 gene is NCBI gene ID: 4360 and GeneCard GCID: GC10P017809. Aliases include CLEC13D. In assembly GRCh38.p13, the human MRC1 gene is located at Chr 10: 17809348 . . . 17911164. The MRC1 gene is conserved in chimpanzee, Rhesus monkey, dog, cow, mouse, rat, chicken, zebrafish, and frog.

Regulatory features which are present in the MRC1 gene may be identified by any suitable method known to the skilled person. For example, regulatory elements can be identified in GeneHancer which is a database of genome-wide enhancer-to-gene and promoter-to-gene associations. Regulatory features which are present in the MRC1 gene include a MRC1 promoter, a MRC1 enhancer, and MRC1 5′ and 3′ UTRs. Mannose receptor regulatory sequences are located, at least in part, immediately upstream to the site of transcriptional start (Eichbaum, Q., et al., Blood, 1997. 90(10): p. 4135-43).

### Phagocyte-Specific Promoters

The vector of the present invention may comprise a phagocyte-specific promoter, particularly a liver and/or splenic phagocyte-specific promoter. Suitably, the transgene is operably linked to a phagocyte-specific promoter, particularly a liver and/or splenic phagocyte-specific promoter.

A “promoter” is a region of DNA that leads to initiation of transcription of a gene. Promoters are located near the transcription start sites of genes, upstream on the DNA (towards the 5′ region of the sense strand).

As used herein, a “phagocyte-specific promoter” may be a promoter that enables phagocyte-specific expression of a transgene which is operably coupled to the promoter

Exemplary phagocyte-specific promoters include a MRC1 promoter; an ITGAM promoter; a CD86 promoter; a CD274 promoter; a CD163 promoter; a LYVE1 promoter; a STAB1 promoter; a ITGAX promoter; a SIRPA promoter; a TIE2 promoter; a CHIL3 promoter; a CD68 promoter; a CSF1R promoter; a VCAM1 promoter; a PTGS1 promoter; and a C1QA promoter.

An engineered promoter variant derived from any of these promoters may be used, provided that the variant retains the capacity to drive phagocyte-specific expression of a transgene which is operably coupled to the promoter. A skilled person will be arrive at such variants using methods known in the art. The variant may have at least 70%, at least 80%, at least 90%, at least 95%, or at least 99% identity to any of the promoters.

A fragment of any of these promoters (or variants thereof) may be used, provided that the fragment retains the capacity to drive phagocyte-specific expression of a transgene which is operably coupled to the promoter. A skilled person will be able to arrive at such fragments using methods known in the art. The fragment may be, for example, at least 200 nucleotides, at least 300 nucleotides, at least 400 nucleotides, at least 500 nucleotides, or at least 1000 nucleotides in length.

In some embodiments, the phagocyte-specific promoter is selected from the group consisting of: a MRC1 promoter; an ITGAM promoter; a CD86 promoter; a CD274 promoter; a CD163 promoter; a LYVE1 promoter; a STAB1 promoter; a ITGAX promoter; a SIRPA promoter; a TIE2 promoter; a CHIL3 promoter; a CD68 promoter; a CSF1R promoter; a VCAM1 promoter; a PTGS1 promoter; and a C1QA promoter; or a variant and/or fragment thereof.

In preferred embodiments, the phagocyte-specific promoter is a MRC1 promoter or a variant and/or fragment thereof.

### Mrc1 Promoter

In one aspect, the present invention provides a vector comprising an MRC1 promoter. Suitably, the transgene is operably linked to an MRC1 promoter.

Any suitable method may be used to identify an MRC1 promoter, for example by using promoter prediction tools or by using a sequence immediately upstream to the MRC1 open reading frame. Suitably, an MRC1 promoter may be about a 0.2-5 kb, 0.5-5 kb, 1-2 kb, or about 1.8 kb sequence immediately upstream to the MRC1 open reading frame.

In some embodiments, the MRC1 promoter comprises or consists of a nucleotide sequence which is at least 70% identical to SEQ ID NO: 1 or a fragment thereof. Suitably, the MRC1 promoter comprises or consists of a nucleotide sequence which is at least 80%, at least 85%, at least 90%, at least 95%, at least 98%, or at least 99% identical to SEQ ID NO: 1 or a fragment thereof.

In some embodiments of the invention, the MRC1 promoter comprises or consists of the nucleotide sequence SEQ ID NO: 1 or a fragment thereof.

Exemplary human MRC1 promoter
(SEQ ID NO: 1)
CCCTGAATGTGATTATATACATAATTCAATTAAATGTATTTGCTT
CTGAAATATATATAAATGTAAATTAGGCAGTCACTTTTGTATATG
ATTTATTTATATTTGAAAGCCACAAATGACCCATTTAAACTATTA
TTTTCATAAGCCAGTGAAACAATGTCTGAGAAACATTTTTGTTTT
GTCTGTTCTGTTCTATAACCATCATTTTTTTTTTCAGTCATGTAC
AGCCTTAGTGACAAAGAAACTTTGGTCCTCTGTCCTACATTTTCA
CTATCTTTTTCCCTCCGGTCAGGATAATCTCAAATTTACATGTTA
AAAACAATCAGTAAGAGAACTACATCACATTTCTAATAGGATGGA
AACTTTTCAACTTTATCACAAAGACAACGAATGTGGAGGCTTTCC
GTTTGAAGATAAAACTATTCATTTAAAAAATTTTAAAAATTACAA
TGTTTCCAGTAGCTTCTTTTTGAATTACTAACATATTCCACACTC
TAGTAACGGTTTGGCCAGCTAATCGTTAGTTTCTGCTTTAAAATG
TTCTAAATTCCTGTTCTACTTTTGAAAAATGACAACATAAATGTT
TGGAGGGTTATTTTCTGCTTAATGAAAGATCTAGAAACATATTTT
ATTCTAAGAAAGAATTCCACTTGCCTTTAAATAAAGATATACCTT
TTGACCAAACAATCAGATTTTCTTTTTCTTTTTTTTCTTTTCTTT
TTTTTTTTTGAGATGGAGTTTCGCGTCTGTCGCCCAGGCTGGAGT
GTAGTGGTGCGATCCTGACTCACTGTAACTTCCACTTCCCAGGTT
CAAACGATTCTGTTGCCTCAGCCTCCTGAGTAGCTGGGCTTACAG
GTGTGCATGATCACACCCGGCTAACTTTTGTATTTTTAGTAGAGA
CGGGTTTTTGCCATGTTGACCAGGCTGGTTTCAAACTCCTGACCT
CGGGTGATCTGACTGCCTCGGCCTCCCAAACTGCTGGGATTGCAG
GCGTGAGCCATTGTGCCTGGCCAGATTTTCTTTTTCTAGCAAGGG
GACCCACTTAAACTTGAAGAGGACCGGGATGGTTGAGGCTGGGCA
GCAAGGCTTTACTGCAAATCCTTTACCACTGTTTTTTCTGGCTTT
CTAGAGAACGTTCTAGCAAAAGGTTTCTAGAACTTTCTCCTTCCT
GGCCTGACTGACATTCCCTCTTAGGTGTAGCCTCCTTTTCACTTT
TCTTCTGCCTGGAGGAAATGAAGCTCCACGGAACTTTCTGTTGAA
ACTTTCCAAGAAAAAAAAGAAAGGCTCTAAGCACTGAATGTGGAA
ACTGAAGGGGATGAGCTTCAACTCTGAAGTGTTTCCAGCGTAAAA
CTGTCCTTTCCAGGGCCCGTGTGGCTGTCACTTCAGAGTGGAGGT
TGTCTGCTGAGGGACCCCTGACTCAGCTGCTTCCCAGGGGAAGCT
CCGTCTTCCGGCACAGGTAATGGCCTGCAGCTTGATCTCCACCCA
GCCCCATCTGAGCAGGCCGGGAGCTCCCAGGCTGTTTCACTTCTC
TCCTTCCTGACTCCTCACCATCACCATCGCCCTCTCTCCTCCCCA
CCCCGCCACTCCTCTCCCACACGTGTCCCTTTCTCCCCTTCCTCT
GCGTCTGCTCTTCTCAGAAGTTAGCTTACGAAGCAAAGTTGTTAC
TTTGAATTCCTGTTTTTCCAGCCACCCTCATGTGACAGGATGTCT
CCTCAGTAGAGGCTTTCCCTAAATTCAGGAGCCCTTTAAAAGGGA
GGGCTTCCTCTGTAGTTCTTTTCAGCTGGGCAGCTCTGGGAACTT
GGATTAGGTGGAGAGGCAGTTGGGGGGCCTCGTTGTTTTGCGTCT
TAGTTCCGCCCTCCTGTCCATCAGGAGAAGGAAAGGATAAACCCT
GGGCC

In some embodiments of the invention, the MRC1 promoter comprises or consists of a nucleotide sequence which is at least 70% identical to SEQ ID NO: 2 or a fragment thereof. Suitably, the MRC1 promoter comprises or consists of a nucleotide sequence which is at least 80%, at least 85%, at least 90%, at least 95%, at least 98%, or at least 99% identical to SEQ ID NO: 2 or a fragment thereof.

In some embodiments of the invention, the MRC1 promoter comprises or consists of the nucleotide sequence SEQ ID NO: 2 or a fragment thereof.

Exemplary mouse MRC1 promoter
(SEQ ID NO: 2)
CTCGAGCCGAGCTCTGAAATGGATGCTTCAAGGATTTGAAGAGAC
ACCAGAAGTGAAAAACGTGCTATTTTCCCACAGTTCCTGGCAATA
CAAAGATTGTTTTAAGGCCTATGGAAATTCCTCTTCCTCCGTTAC
CTGAAATTACAGATTTGTGTTGACTTGCTCACCCCTCCTAACCTG
ATAAAATCTTCCAATAAGATAAAAATGATGGAGACAAATCCTTTG
TGGGATGTTGGACTTCACTTTATATCACATCCAGCGTCTCGTTAC
TGATTCTGATTTTATTCCTGTGCATGTAAGACACGTTGACATAAT
AAAACCATGGATATACAGATGCCTGCAATTCAGTTAACTCTTTTT
TTTCCTCTTCAAATAAGTCAAAGCAAACCCCAATTAGGCAAAACA
ATTTGAATGGCTTGCATTTAAAAGACCAATTAAAACATTTTTTGG
TCAGCAAGCATGATGGGACACACTTATAATCCCAGCTCTCAGAAA
GTCAAAACAGAGGAACCAAGAATTCAAGGCCAGCCTGCGCTACAA
ACGCAAGACTGTTTCGGTGTTCCTGTGATAAGTCAGTTACGCAGT
GATTGAAAAGGAAACGTTTGCAGCCTCTCACCAGTTGTGGGAGAA
TTTTCTTTGTCAGTTAAGCCTTGATAGAATGAAAAAGAACGGTGG
GTCCCTTCTCAGAATCTTCCTAATTTAGGCTTTTTAAAAAGAAAA
TTCTTGAGAGAAACCACAGCTTATTGGGAAATGAGTGTGTACCTG
CCTCAGCGTGGATGGGTCTGAACAGCTTTTCACTTGAAGGTAAAC
CATCTGTTTACAACTTCTAAGTCGCCAGTGTTTCCAGAGCTTCTT
TTTGAAACGATGACATTTCCCACGCTCCAGTTTCAGGTCTTCCCT
GACTAACCACAAATATCCATTTCTAAATATTCTTAATTCTTGTTG
AACGTCTGGAAAAAAAAAATCAGTGTTTAGGTGGGTTGTGTGGTG
CTTTGTGAACGACCCTGCAAAATCATGAAGACGAAACCCCACTGT
CATCGAATCAACAAGCAACTTTTGGACTCAAGCCAGGCTTTCTTT
TGCAAGAGAGAGAGAGAGGTCTTCCCTTTTTCAAACTCTGAGGAC
TGTAATGGTTGAGGCCTGGCAGCGAACCGACAACAAAGCTATTGC
CACTATTTCCTCTGGCTTTCTAAGGAAAGCTGCTAGAACTTTCTA
TCCCTGGGCTTCATTGAGGTTGTCTTAAAATTAACTTCTGTCATT
TTCCTTCTAGAGACAGGGGCAAAACTCTACGTGAACCATACCTTT
GATCCTTTCCAAGGAGTGTGTGTGTGTGTGTGTGTGTGTGTGTGT
GTGTGTGTGTGTGTGTGTGTTGGTGCTCGGGCTCTAAGCCTGAGC
AGGAAGAGCTTCTGATGCTTTCCAGCGAGTGTCCTCCCTTTCTGA
CTGTAGAATTGTGGGTGAGAGCCTCCACAGCTGCCTCCTGGAGAC
TTTTTCCCACCCAGATAATGGCCTCCGTTTGGTTACTGCCCAGCA
CCTGTGGAGAGCTCAGCAGGGCTGCCTCTCCCTGCTGCTCATGGC
CTGGGTCCTCACTTCTCCCCACTTCCTGCGTTTTCTCCTCTCCTA
CACATGTTCCTCTCTCCCCTTCCTCCTGTGCCTTAGCTTACGAAG
CAAAGTTGTAACTTTGAATTCCTGTTTTTCTAACCGCCCCCATGT
GACAGGATATCTCTCAATTGGAGGGTTTTCCTAAATTCAGGAGTC
CTTTAAAAGGGACAGCTTCCTCTGTCCTCCTTTTCAGTCAGGCAG
CTCCCAGACCTTGGACTGAGCAAAGGGGCAACCTGGGGACCTGGT
TGTATTCTTTGCCTTTCCCAGTCTCCCTCTTCTCCCTCATTGGAA
CCGGT

In some embodiments of the invention, the MRC1 promoter comprises or consists of a nucleotide sequence which is at least 40% identical to SEQ ID NO: 1 and SEQ ID NO: 2 or a fragment thereof. Suitably, the MRC1 promoter comprises or consists of a nucleotide sequence which is at least 50%, at least 60%, or at least 70% identical to SEQ ID NO: 1 and SEQ ID NO: 2 or a fragment thereof.

In some embodiments, the MRC1 promoter comprises or consists of a nucleotide sequence which is at least 70% identical to SEQ ID NO: 31 or a fragment thereof. Suitably, the MRC1 promoter comprises or consists of a nucleotide sequence which is at least 80%, at least 85%, at least 90%, at least 95%, at least 98%, or at least 99% identical to SEQ ID NO: 31 or a fragment thereof.

In some embodiments of the invention, the MRC1 promoter comprises or consists of the nucleotide sequence SEQ ID NO: 31 or a fragment thereof.

Exemplary XhoI-Human.MRC1.promoter
(SEQ ID NO: 31)
CTCGAGCCCTGAATGTGATTATATACATAATTCAATTAAATGTATTTGCT
TCTGAAATATATATAAATGTAAATTAGGCAGTCACTTTTGTATATGATTT
ATTTATATTTGAAAGCCACAAATGACCCATTTAAACTATTATTTTCATAA
GCCAGTGAAACAATGTCTGAGAAACATTTTTGTTTTGTCTGTTCTGTTCT
ATAACCATCATTTTTTTTTTCAGTCATGTACAGCCTTAGTGACAAAGAAA
CTTTGGTCCTCTGTCCTACATTTTCACTATCTTTTTCCCTCCGGTCAGGA
TAATCTCAAATTTACATGTTAAAAACAATCAGTAAGAGAACTACATCACA
TTTCTAATAGGATGGAAACTTTTCAACTTTATCACAAAGACAACGAATGT
GGAGGCTTTCCGTTTGAAGATAAAACTATTCATTTAAAAAATTTTAAAAA
TTACAATGTTTCCAGTAGCTTCTTTTTGAATTACTAACATATTCCACACT
CTAGTAACGGTTTGGCCAGCTAATCGTTAGTTTCTGCTTTAAAATGTTCT
AAATTCCTGTTCTACTTTTGAAAAATGACAACATAAATGTTTGGAGGGTT
ATTTTCTGCTTAATGAAAGATCTAGAAACATATTTTATTCTAAGAAAGAA
TTCCACTTGCCTTTAAATAAAGATATACCTTTTGACCAAACAATCAGATT
TTCTTTTTCTTTTTTTTCTTTTCTTTTTTTTTTTTGAGATGGAGTTTCGC
GTCTGTCGCCCAGGCTGGAGTGTAGTGGTGCGATCCTGACTCACTGTAAC
TTCCACTTCCCAGGTTCAAACGATTCTGTTGCCTCAGCCTCCTGAGTAGC
TGGGCTTACAGGTGTGCATGATCACACCCGGCTAACTTTTGTATTTTTAG
TAGAGACGGGTTTTTGCCATGTTGACCAGGCTGGTTTCAAACTCCTGACC
TCGGGTGATCTGACTGCCTCGGCCTCCCAAACTGCTGGGATTGCAGGCGT
GAGCCATTGTGCCTGGCCAGATTTTCTTTTTCTAGCAAGGGGACCCACTT
AAACTTGAAGAGGACCGGGATGGTTGAGGCTGGGCAGCAAGGCTTTACTG
CAAATCCTTTACCACTGTTTTTTCTGGCTTTCTAGAGAACGTTCTAGCAA
AAGGTTTCTAGAACTTTCTCCTTCCTGGCCTGACTGACATTCCCTCTTAG
GTGTAGCCTCCTTTTCACTTTTCTTCTGCCTGGAGGAAATGAAGCTCCAC
GGAACTTTCTGTTGAAACTTTCCAAGAAAAAAAAGAAAGGCTCTAAGCAC
TGAATGTGGAAACTGAAGGGGATGAGCTTCAACTCTGAAGTGTTTCCAGC
GTAAAACTGTCCTTTCCAGGGCCCGTGTGGCTGTCACTTCAGAGTGGAGG
TTGTCTGCTGAGGGACCCCTGACTCAGCTGCTTCCCAGGGGAAGCTCCGT
CTTCCGGCACAGGTAATGGCCTGCAGCTTGATCTCCACCCAGCCCCATCT
GAGCAGGCCGGGAGCTCCCAGGCTGTTTCACTTCTCTCCTTCCTGACTCC
TCACCATCACCATCGCCCTCTCTCCTCCCCACCCCGCCACTCCTCTCCCA
CACGTGTCCCTTTCTCCCCTTCCTCTGCGTCTGCTCTTCTCAGAAGTTAG
CTTACGAAGCAAAGTTGTTACTTTGAATTCCTGTTTTTCCAGCCACCCTC
ATGTGACAGGATGTCTCCTCAGTAGAGGCTTTCCCTAAATTCAGGAGCCC
TTTAAAAGGGAGGGCTTCCTCTGTAGTTCTTTTCAGCTGGGCAGCTCTGG
GAACTTGGATTAGGTGGAGAGGCAGTTGGGGGGCCTCGTTGTTTTGCGTC
TTAGTTCCGCCCTCCTGTCCATCAGGAGAAGGAAAGGATAAACCCT

### Inducible Promoter

Suitably, the phagocyte-specific promoter may be an inducible promoter

As used herein, an “inducible promoter” is a promoter which is only active under specific conditions. For example, expression of the transgene may be induced by a small molecule or drug (e.g., which binds to a promoter, regulatory sequence or to a transcriptional repressor or activator molecule) or by using an environmental trigger. Types of inducible promoter include chemically-inducible promoters (e.g. a Tet-on system); temperature-inducible promoters (e.g. Hsp70 or Hsp90-derived promoters); and light-inducible promoters. Suitably, the promoter is chemically-inducible.

Any suitable method for engineering an inducible phagocyte-specific promoter may be used.

Alternatively, the phagocyte-specific promoter may be a constitutive promoter. As used herein, a “constitutive promoter” is a promoter which is always active.

### Phagocyte-Specific Enhancers

The vector of the present invention may comprise a phagocyte-specific enhancer. Suitably, the transgene is operably linked to a phagocyte-specific enhancer.

An “enhancer” is a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site.

As used herein, a “phagocyte-specific enhancer” may be an enhancer that enables phagocyte-specific expression of a transgene which is operably linked to the enhancer.

Exemplary phagocyte-specific enhancers include a MRC1 enhancer; an ITGAM enhancer; a CD86 enhancer; a CD274 enhancer; a CD163 enhancer; a LYVE1 enhancer; a STAB1 enhancer; a ITGAX enhancer; a SIRPA enhancer; a TIE2 enhancer; a CHIL3 enhancer; a CD68 enhancer; a CSF1R enhancer; a VCAM1 enhancer; a PTGS1 enhancer; and a C1QA enhancer.

An engineered enhancer variant derived from any of these enhancers may be used, provided that the variant retains the capacity to drive phagocyte-specific expression of a transgene which is operably coupled to the enhancer. A skilled person will arrive at such variants using methods known in the art. The variant may have at least 70%, at least 80%, at least 90%, at least 95%, or at least 99% identity to any of the enhancers.

A fragment of any of these enhancers (or variants thereof) may be used, provided that the fragment retains the capacity to drive phagocyte-specific expression of a transgene which is operably coupled to the enhancer. A skilled person will be able to arrive at such fragments using methods known in the art. The fragment may be at least 200 nucleotides, at least 300 nucleotides, at least 400 nucleotides, at least 500 nucleotides, or at least 1000 nucleotides in length.

In some embodiments, the phagocyte-specific enhancer is selected from the group consisting of: a MRC1 enhancer; an ITGAM enhancer; a CD86 enhancer; a CD274 enhancer; a CD163 enhancer; a LYVE1 enhancer; a STAB1 enhancer; a ITGAX enhancer; a SIRPA enhancer; a TIE2 enhancer; a CHIL3 enhancer; a CD68 enhancer; a CSF1R enhancer; a VCAM1 enhancer; a PTGS1 enhancer; and a C1 QA enhancer; or a variant and/or fragment thereof.

In preferred embodiments, the phagocyte-specific enhancer is a MRC1 enhancer or a variant and/or fragment thereof.

The vector of the present invention may comprise a phagocyte-specific promoter and/or a phagocyte-specific enhancer, i.e. a phagocyte specific promoter and/or enhancer. Suitably, the transgene is operably linked to a phagocyte-specific promoter and/or enhancer.

In some embodiments, the phagocyte-specific promoter and/or enhancer is selected from the group consisting of: a MRC1 promoter and/or enhancer; an ITGAM promoter and/or enhancer; a CD86 promoter and/or enhancer; a CD274 promoter and/or enhancer; a CD163 promoter and/or enhancer; a LYVE1 promoter and/or enhancer; a STAB1 promoter and/or enhancer; a ITGAX promoter and/or enhancer; a SIRPA promoter and/or enhancer; a TIE2 promoter and/or enhancer; a CHIL3 promoter and/or enhancer; a CD68 promoter and/or enhancer; a CSF1R promoter and/or enhancer; a VCAM1 promoter and/or enhancer; a PTGS1 promoter and/or enhancer; and a C1 QA promoter and/or enhancer; or a variant and/or fragment thereof.

The phagocyte-specific promoter and the phagocyte-specific enhancer may be a combination of any of the above, for example a MRC1 promoter and an ITGAM enhancer.

In preferred embodiments, the phagocyte-specific promoter and/or enhancer is a MRC1 promoter and/or enhancer or a variant and/or fragment thereof.

### Exemplary MRC1 Enhancers May Include:

Mouse Mrc1 enhancer 1
(SEQ ID NO: 17)
ACAGAACCAGCAGTATAGGGAAGGCCGTGGTGTTGTGGGACTCACATGATATTATTTATGATATCTTGGAAA
TTAGAGCAAAGACAGGTTAGGCATTGTGGTCAGAGGAGCTGGGTTATGACACCGAGGAAACAAGCTGACCCT
TGAATTAAAACATATTGACGCCATAGCAATAAGAGGATGGAACCACATTGCCCTCTGCTGTTGGGGAATCAT
GGCCGCTGCCCCCATTCTGCAGTTAAGAGACCCGGTACTGCCCTCTGCTGGCTGGATGCACATGTTTCCACA
TTCTGGATTAGTATCCTTTTGAATTTAAATTTAAAAACAGTCTCCTGCTGCCTGCCAGTGACTCACTGTGGC
CTCTTTATGTTGTTAGTAGCTTTGTTTTACTCTGGCAGATAGAAAATATGTTACAGGTCGCCATCTTGGTTC
CGGGACTCAGCA
Human MRC1 enhancer 1
(SEQ ID NO: 18)
AGCCCCACCATGTTATTGATGGCCAAACAATACGCATGCTGACAGCCATTATCTGTGGCCTCTGATGCTATT
AGCCAAACCATGTTATTGATGGTCAAACAATACGCATGCTGACAGCCATTATCTGGGACTCAGAAAGTTCTG
CATATTCAAGTCAGGCCAGAGGATCCGAGTTCTAATGTTAAGAGAAACCAACACACCAACAAGCAAATAAAC
AAACCTACCCTTGAACCAAAATATACATCAATACCTCCGTTGCAAATGGATAAATGGAACTGCATTGCCCTC
TGCTGTTGGGGAATCTTGGCAACCATTTCAACTCTATGGCTGGAGATGACTTACTGCTCTGTTTATTTTCCA
TCCTCCTGCTTAGATTATTGCTTTCAAAGTTTCCAGAATAGAAGAAGTCAGTGGTGGCCAGTTGTCCTTTAA
TGGTCTCTTATCTACCAATGGCTAGTATCCTTTTTGCATTATCGTAGCTCTACTCTTGTAGATGTTAAATT
Mouse Mrc1 enhancer 2
(SEQ ID NO: 19)
ACATGGGAGGCAAGGCGGAAGGAGCATGAGGCTGACCTAGCAGGCAGGAAGCACAGAAATCACATTTTGAGC
TACATAGAAGAAGGAGAGAGAGAGAGAGAGAGAGAGAGAGAGAGAGAGAGAGAGAGAGAGAGAGAGAGAGAG
AGAGAGAGAGAAATCAGGAAGTGAGACTAGTCTATAAAACTGCAAAGCCTACTCCCACTGACATACTTCCTT
TAGCAATGCACAGCTGCCACAACCCTCCCAAATCCTGCCACCAACTGGAGACCAAGGGTTACAATAAGTAGA
CCTAAGGGAGGGGTACTTTTCTTTTCAACCACTGCAGTGGAGCACACCTCTATGTCCAACATGAAGGAAATA
GAGGCTGGAAGACCAGAAATTCAAGGTCACCCACCAGCTCATCGCCAGTTGCAGATCAGTTTGAGCTACAGG
CTATCTGCCTCAAATATAAAACTAAACAGAAAGTCAATAAAAAGGCCACACTTGGGGAAGTGGATAATAGGG
TCAAATATTAGTAAACACCTCTTCTTCCCCATTGTTAAAGCCTGCTCCCTCCAGTTCCTCTGACTTTACTGT
TACATAACAGATCTTGGACCTGTGACTGCTGTGTTTACAACATACTCAGTGACCCCTAACTTCTAATCATGA
AACACATTTACCCGGTTCCAGGATGCCATCTCTCCACCTACAGCTCACCATGGAAGCATTTTGCCTCTTAGC
AAAGGTCTTTGGTTTCTCGTGGGTGGCA
Human MRC1 enhancer 2
(SEQ ID NO: 20)
TTTATTAGATTGTGAAGACTAAAAGGGCTAAAGTTTGCTTGAGAAGAGAGTATCCGTTTTAGCTTCTAAAAT
ATACACTTGGGGAAACAGAGAGATTAGGATCAGAAATTAGTGTGGGAGCAAAAGCATCCCCGATTTCTTGCC
CCCAAAATCTCCTCCCTCCAGTTTTTCTGGACACGGCTATATTAATATTAGTCTGTCTCTGCTCCTATGACT
GCCATGTTCAAAAAACCCCTTGTGGCTTCTCATTTCCCATCAAATAATTAACAAAATACATTTTGCCTGACA
TTCAAGATTGCCACCTGCTGAACATCTGTACTATTCCCGGTCTTGCATTTTGCGGTCACAACAAAAGAAAAG
TCAGTCTTGGATTCCTTGTTTTTCCTCATCCATTCTCACCCTCACATCTTTTCCCATGTCATTTCTTTTGCC
TGGAATTCTCACGATTGTGTACAGTTGAAATGCAATTCTATTTCCATCTGCATTTCTCTTATTTGCTTATTA
CCACTTTTATTAGTATAATAGCTACTTACACATATGTCCTGCTTCCTTGACAACACTATAAATACATTAATT
TGTTATTATTTATTTACTTATTCATTCATTCCCAAGATATCTTGTACCCATGTGCCAGGCTTTGTGCTAGGT
CCTGGGGACATCAAAGCTGACCCTGTCTTGTCCACATGCAGCTTAAAAGTCCAATGGGAAGAAACAGATGTA
AACAAGAAAACAAACAAATGAATAGGTACCAATTATAATAAGTCCTGCATGGAAAGCAAACTGAAGGCCATG
TGGAGATCGCGTCAGAGGGGGAAATAGCTGAAAACGGGGAATGTCTTGTGAATATTTATGACCCCTCTTCCC
CCAAGGAGGTTCACACAGAGCCTTCCAAGGAAGGGTCTCACCAAACATTTGCTGACTGACATGTTAACATGC
AACAAAATAAAAATACTTAGAAATTCAGTCTCCTGTTTGGAAAACTAGACAGTCATGGCAAGAAGACAGTAA
ATGCAGTGGTTCTTCTGTGTGAAGTGGCTTATTCATCACCTTGGAATTCTGTTAAAGTGATGATGCCTGGGC
TGAGATCCCGAAGACTCTAGATCAGTAGGTGTGACACAGGGCACAAGAATCTGCCTTTTGTCAAACTCCTGG
GTGATTCTGGGGCAGCTGGTTGAAAGACCATATCTGGCAAAACACTGTGTTAGTTCCTGCTGTATCCAGGTG
CTAACACCAGGCAGCAGAGCAGAGAGGCTGAGAGCAAAGATTCTGCAGCCAGACTGCCTAGGTCCCAGCTCT
GTGAA
Mouse Mrc1 enhancer 3
(SEQ ID NO: 21)
GGTCAACTATATAGTAATGAACACCTATCAATTATTTTCCTTAATATATTAGATTTTATTCCTCTTAATTCA
GCATCACTTGCATTCTAATGAAGATCTCTATGTCCTTCCAGCCATGTACTCCTTACTGGGCAATGCAAATGG
AGCCGTCTGTGCATTTCCATTCAAGTTTGAAAACAAGTGGTATGCAGACTGCACCTCTGCCGGGCGCTCGGA
CGGATGGCTCTGGTGTGGAACCACCACTGACTACGACAAAGACAAGCTGTTTGGATTTTGTCCATTGCACTG
TAAGTAACTGAAAACAGCACACCTGGGACATTCAGTATGGTCACATGATGGTAGGGTGGACTTTATGTACCC
TCTATCTACCTTTCTTTGTTTCTTGTTTCACTTTCACTTCTCTCTCTCTCTCTCTTTCCTTCCCCATCTTTC
TGTTTGCTAAGGATCAAACCCAGCCTTGCACATGCTAAACAAACACTCTACTACTGAGCTACATATCCTGAC
CTTATTAGTTATTTGCTAAGACTTTAGGGCAAATTATACTGAATATAGCATTATATATAGTCAGTGCTGGAG
GTAGGTACATCGTTCTCCCAAACCCCAAGTGTTTTAGTTTTAAAAAGCCATAGGTTAAAGCAGGAATTTAAA
TAACACCACAAACGAGTTTGGTGGGAGTCTGTGAAGGCTCTTGCATTTTGCATCACCATGTGCTGGAGCTCC
TTCTAGTACAGATGATACTGATGGAGGTTTGTGAATCTCAACGTAAGAAGGGTGGAATTCAGCTGAGTTGGC
AATCAAGGGAAGTGAGTAATCTATGCTTCAGTCCTTTGAAAGCAGAAGTTTGGTGTTACTAGAGCATGCAAG
ACCACATAAAGTACCAGAACTTGAATTCTTGAGGTTTTATCCATTCGTAAGAATCTGTAAGAAAATATGTGG
CAGCTTAGGTGGGGCTAGGGAGGGCAGCTGGGAGTCAGAGCTAGGGCTGAGGGAGGAGAAGGTTGAGGTCTT
GGCTTAACTTCTGTATCTCTGAACATACTTTCTGAA
Human MRC1 enhancer 3
(SEQ ID NO: 22)
CAGCCTCCCGAGTAGCTGGGACTACAGGCGCCCGCCACCACGCCTGGCTAATTTTTTGTATTTTTAGTAGAG
ACGGGGTTTCACTGTGTTAGCCAGGATGGTCTCGATCTGCTAACCTCGTGATCCGCCCACCTCGCCTCCCAA
AGTGTTGGGTTTACAGGCATGAGCCGGCGCGCCCGACTATGACCTCTTTATTTTTACCAACTATCAGTTACT
CAGCGAAAATTATCCTGTACACAATATAGTATATACTTAACACGGGAGGTAGGTAGGCATGCTATTATTCCC
TCATCTCTGAAATGCCTTACCTTTATGAAACCATAGATCAAAAACAGAATTCAAAGGAAGCCACAAGCATGT
GGTTTGATGGAGAAAGACGTGAAGGTCCTGAATTTTGTGTCAATGTCATCAAAAGTGTTCTTCACAGTGAAA
ATGATAGTCAGGATACTCTTCTTATGTTTAAATTAAGAAGGGTGCCATTTAGTTTCACACAGCTAGAAATCA
ATGGAACTCGGTAATTTATATGCTTCAAGGTTTCTTAAAAATGAAAATTCAGGTTTTATTGCAAGGATGCAA
AATAAGCAGTATCGCATTATCTTAGTGGGAAAGCACCAGAACAGATGAATTCTTGGTGTTCTTCCACGCTTA
AAAATCTGTAGGCAAATAGTGAAGCACATTTTAGGTGGAGTGGAGGAGGGCGTCATGTTGAAGGCAGAGCTG
GGGCTGAGGAAAAAGGAAAAGGAAGTATTCCTCTTAGCTTCACGTTCCCCATCACCAGACACCCTCCTCCTG
ATGCTGGCTCCACCCTTCCCAAACTTCTTACCCCCGACCTCTCACCTGCTACTTTAGACCAGATCAGAGTAG
CTCTTGTTTCCTGGTTATCACCCAGAACTCTTTCTCCTGCTGCCCTGCAAAGGGACTGGGCAGAGCAAAGAG
CATTCGATATGGTCTGGGATGATTGTGACACCACCTGAGTAACAATAGAATCACGACTATCACAACTCAACT
TTCCAGACCACAAATCCACAAGTAATCACACTATTTCAAGCATTATTGTAAAACAGAACAACTTAAAAAATA
CCTGAATTTGACGAACAAAAGCCAGAATTCTAAGAATTGTACTTATTTATCTCTCTGGATTTATAATCCCTA
ATTATCACACTAAAAGTAAATTTAATTTCTGAGCCCCATATACTATTGTAATTGTCTTCAGAGTGCAGTCTC
TCCAATCCGAATGAATACTCACAAAAGCCCATAGGCTTTCTGTTCATAGCGACACTGCTGCCTCGGTCTTAA
CTGAGGTAGTTCTATTTGTCTCTCTTATGTCAATCTTTAGAAAGACATTTGATTTCCATTCAAGGTTTTTAG
ATGTCGAATTTTGTATTCGAAGTATTTTTGTCTGAAACACATTGAGCAATTTTTTTCTAAGATAAAGCAATA
CTTGGTTTTCAAGTGATTGAAAGTGTCTTTCTCCTTTACTTAATAGGAATGATATTTTCTTAATCTGTTTCA
TGGACTTTCTTAAGGGTATATATTTCATGGGTCCAACTATAGCATCCTCCACATCCTTTGAAATTGACAAAG
GAGTTAGATGAATGTGTGATTTCCTGAATGAAATGTGGAGGACAAGTGGTAAGTTACTAATCACAAAGAAAA
CTCACAATCTTGGAAATCCTTGGATGTGTGTTGGAGACGTATCTTGAGTTTGTTCAGTGGAATAATTTTTTA
GTCTTATTACTTGTATTTATGCCTTCACTGTCAAATTATATATTTTTTCCTGTTAAATGTAAAATAATCGTA
GAAAATAAATTGATTTGGTTTCAATATGCATTAAAATTTTAAATCACGTTTTGTACATTTAATATCTTTCTT
AAAGGGCTTTATAGTCTTCCAGTCTGTTTCATTTTGTGTTCTTTTCAAAAGAGTTTTTATTGTATTTATTTA
TTTATTTATTTTTGAGACAGGGTCTCACTCTGTCATCCAGACTGGAGTGCATTAGCATGATCTTGGCTCACC
ACAACCTCTGCCTCCCAGGCTCAAGTGATTCTCCTGCCTCAGCCTCCTGAGTAGCTGGGAT
Mouse Mrc1 enhancer 4
(SEQ ID NO: 23)
AATAAACGTCTAGGAACATTTACCCTAAAGTACTGCCCTCTCTATGTGAACAAACTTAAGCCTGTGTTCTTT
CCTTTTTGTGAACAGACGCGAGGCAATTTTTAATCTATAATGAAGATCACAAGCGCTGCGTGGACGCTCTAA
GTGCCATCTCAGTTCAGACGGCAACTTGCAACCCGGAAGCTGAATCCCAGAAATTCCGCTGGGTGTCAGATT
CTCAGATCATGAGTGTTGCTTTCAAATTATGTTTGGGAGTGCCATCAAAAACTGACTGGGCTTCCGTCACCC
TGTATGCCTGTGATTCGAAAAGTGAATATCAGA
Human MRC1 enhancer 4
SEQ ID NO: 24)
TGGAAGAGTTGGAAACTTTTGACCTAAAAGATCGTCCTTGTTACATGAATCCACTTAGCCATGCTTGCTTTC
TTCTTCTTTTCCTGCTTCTTTCTTTTTAAACAGACACCAGGCAATTTTTAATCTATAATGAAGATCACAAGC
GCTGCGTGGATGCAGTGAGTCCCAGTGCCGTCCAAACCGCAGCTTGCAACCAGGATGCCGAATCACAGAAAT
TCCGATGGGTGTCCGAATCTCAGATTATGAGTGTTGCATTTAAATTATGCCTGGGAGTGCCATCAAAAACGG
ACTGGGTTGCTATCACTCTCTAT
Mouse Mrc1 enhancer 5
(SEQ ID NO: 25)
TGTCAGGTTCTCTGGAGCACCCTCTCACCTGTTCAGACTAATTTCCTAAGTTCGGCGGGTCCCGGACCAAGA
TGGCGACCCGCTACATTTCATTCTTACATGCAGGGGATGAGCGCACTGTTTCACCACTTTGATTGCCTTTTT
TGAGCATGGTAGATATTCAGTAAGCAACCCATGGATTGAATTCTACTTTATGTTTAATGCAGGACGAAAGGC
GGGATGTGTTGCCATGAAAACCGGAGTGGCAGGTGGCTTATGGGATGTTTTGAGTTGTGAAGAAAAGGCAAA
ATTTGTGTGCAAACATTGGGCAGAAGGAGTGACTCGCCCACCAGAGCCCACAACAACTCCTGAACCCAAATG
TCCAGAAAACTGGGGTACCACCAGTAAAACCAGCATGTGTTTCAAAGTAAGGATCACTCGCCAAAT
Human MRC1 enhancer 5
(SEQ ID NO: 26)
CATCCTCATTTTATTTTATGTACTTCTTTGTTCGTTAAAGCTGGCATTCCTTACAGTTCTATGAGGCAGGTC
TTGGTATTTGCATTTGGAGAGGAGAAAGCAAGTTCAGAGCGTTTGAGTAACTTACCTAAAATCTCTAGTTGA
GACGTGTCTCATTTTGAAATCTGTGAAAAACTTTGGTCCTGGAAAACCTACGTAGACCTTAGTGGGAGGAAA
AGAATCTTAAAAAATTAAAAAAGAAAGAAAGCAGAGAAAACCAGAAAGGGGAGGAAGGGAAGAAGGAAGGAA
AAAGGGAAGGAAGGAGGGAGGGAGAGAGAAGCAGTAAACTATTTTTGCCATTATGGTGAATTTGATAATATA
AAATATTTTATCATTAAATGCCTGTGTAGGGGGCACTTTGCCAAATGTTAGAAATATAAAGTGTTACAAACC
CCCCTGCATCTGAGATCATAATTGGGCATCAGAACCCTGATGCTCGGTTCTGAGTGCCTTCTGTGAGCACGG
CAGGCCTTCAGCAGGCACCTGTCAAGTGAATTCTACTTCATATATTTAATGCAGGGCGAAAGCCAGGGTGTG
TTGCCATGAGAACCGGGATTGCAGGGGGCTTATGGGATGTTTTGAAATGTGATGAAAAGGCAAAATTTGTGT
GCAAGCACTG
Mouse Mrc1 enhancer 6
(SEQ ID NO: 27)
GAGTGATTGTGCATGAACTTGTGGAGACCTCAATTGTTCTTGCAACTTGTCTCTTCTATTACTATTGCAAAA
GGAATGGCTAAGTCTTTCTTGAAAGAATTCATATAGTTCTCTTTCAGAGACCTGCAGCAGTTACCACTTTGG
GGAACTAGAGAAAAGTTATTTTTAAGTTTCTCTGGAATGAAAGGCACAATTCTATAATTTGGCCTTATTGCT
TAATCCACCAGTTTTAAGTTCCTTGTTTGTAAAATATGAATGTTAGTAACTCTTCTTCTTTAAAATCTCGTT
ATATCATCAAGCTTG
Human MRC1 enhancer 6
(SEQ ID NO: 28)
TTTGGCCAAGATCCTAAATAGATATAGATGCGGGACCTGGATGTTGGGTTTGATTATCCTTTACAGGCTCTC
CATAGTGACGGTGGGTATCTTTAGAGAAAGCTCACCATTTTTGCATTTTACCTCTACTATTCCTCCTCTAGG
AGAAATAGTGTATTTTTTCCTTTTTTGGAAGCCTTCATTACAATTCTCTTTCTAAGACTTGGAATTTCCATG
TTGCCAAAGAGGAGAATAGTTACTTTATAGTTTCTCTGGTACAGCACTCAGAATTCTGTAACTTGGCCTTAC
TGCTTAACCTGCCGGTGCTGGGATCCTCATGTGTAAAATGGAAATGTTCATGACTCTTCTTCCTGAGATAAA
ATTTTGTTCATTTCATCAAACACTCAGTACATTCTTATTCCTCCATGATGCCTTCTTCACTCGCTAACCACT
TGATGTCAGTTTCTGAACATCTCTATACTCCCTGGATTAATGATTCTGTTTTATCTATAAACTCAAATAAAC
CAGAGCTTGGAAAAGCGTATCAGAGTTCAAATTATGCAGCATACGGGATTAGCAACAGCCTTAGGCAAGAAT
TCAACCTCAAACCCTGTGAATTATTGTAACACTAACCCGTTTGTCCATAATCCTCAGGTCTCTAGGGCTGTA
CTCTCTGGCTTAGCAGCCACTTAACCGCATGTGGCCACTGAGCACATGAGCTGTGGCTAGAGGAACAAATCA
TCTTCTGTGGCTGCCCCAGGGAACTCCCCTTCATTTCACTCAAGTTGGTTGTTTTCATGCTTTCAAATATGT
TTAAAGTTCATCATTTCAGTTTTTGAAGGACAGCATTGGCTGATACTATTTTCAATTTCCTAGGTAGCAAAA
TTAAAATAACCCACCAGAGGGCTCCAAAGCTGTACTAAGCTTGCTTTCTTTTTCTTTTTCTTCTTTTTTTTT
TTGTAACCACTGGAAGTGCACAGAATCTAAATTGTATTGAGGGAATAGAATTTTTTTAAATATGC
Mouse Mrc1 enhancer 7
(SEQ ID NO: 29)
ATGAGACGTCTGTCCTGGTTTGAACTTTGCCAACTGAGCCTTATTGCCAGCCTGACTGTTACTAGGAATGGG
TCATGAAATAAATGCTTCTGTCAGAATAGTTTATTCGGATTGAATGTGCTCTGCAACCTCTGCTGACAGCCA
TCTTCCCGAGTGTGTGCAGCAACCAGCCCGAATGTGTCAGCAATGGCTTTCAGGCACCTGTGACACACGTAT
CACAAGTAGGATGTTTTGATGTTTGCAGGGTTATAGCTTCTTCAGGCAAAACTTGCAGGGCATGAAGAAAGC
AGATTCAGCAAGGACCTTAGCCTGAGCAGCTGACTCGAATCGACTGCCAAGTAGCAAGGAATCTGGCACGCG
TTCTGAGCTCCTTGGCCAGCCTGAACCGGCTGAAGCTCAAGCCTCAAGCTCGCCTCTGCACCCCCGCACCTC
CCCCCGCCCCCCACCAGTGCAGACAGTTTTCCTGCTTTTTGTTGTTGTTCCTTTCTTGTTTTATTTAAAAGC
CAGATTCCTTTCATGAAGGGCAGCAAACATGTGAGCTCTGCACATGCGCAGCAGTGAAGAAATTAGCTGAGG
AAGTTGAGGCTGTGTCAGGGCACCTTTCCTGAAGTGGATCCTTGGACATCCAAGCCACTGTGTTCTTTTGGC
CTGTCTTCAACGGAGTACGTTGTATGGTGCCAAGCCTCAGGATACCAAAGAACTGCTTACAAAACACTTGCT
CCTTCACAAGAAGCACAGCAGTTTAGCCAAGATAACATCGCTGCCAAGAACTCTCACATAGGCTAAGATAAA
AACTGAAAGCCCCAGCACATGAGAGTGAGTTCTTGGTAGGAAAAAGCACAGTAAGTTCTTCTCAGCCCTCAC
CGTCAAGATGGCTGGCACGTGCCACCTACTCAGCAGAGATCTGGATGTCTCAACAGTATAGTTCACCTTTCT
GTGTGGATGGGCATCTCCCTGTCCACTGAGGGCCTAGGAAGAACAAAAGCAGCAGCAGGAGGAGTCCACTCT
TTTGCTTCTAGTCTTCCTGTTTAAGCCGGGAGCTTCCCACCTCACTGTCCTTGGAATAGGTTTCACGCCACT
GGCTGGCTTCCCTGCTTTGTGTGTCTTAGAACTTAGATTGAATTCTACCACCTGCTCCTCTGGGTCTTCAAC
TTGCAGGGAGGTCTTGGAATTATCAGCCTTCACAATTTGGCCAGCCTGAACCGCTCAAGCTCAAGCCTCAAG
CTCGCCTCTGCAACCCCGCACCTCCCCCCCCCCGCCCCAGTGCAGACAGTTTTCCTGTTTTTGTTGTTGTTC
CTTTCTGAATTAATTT
Human MRC1 enhancer 7
(SEQ ID NO: 30)
GAAGAAAACACATCCAGTTCTTGGAGGAAAATTGCAATAAATATTTTGAAGAGAGTTCCATCTCTTATTCTC
CCTCAATTTTCTGAAAGTCAGAGTAACACTTGGCTATAAAAGTGATAGGGAAACTAAGTGCCTATCATATAC
CAGGCACAGTGGCATGCAATCAAGTGGGATTTCATGTATTTCCCAAGTGTGTTTTGCTGGCTGCCATGTAAG
ACCCTAGTGTTAATTCCAAAACTCAGAGGTCCTGGCTCTTGAATGGGTGGGGACAGGAGGTGGATTTAAAGT
TTCCAGCAGAGAAGAAGTGTGGGACTGATCGTCTGCTGGAACCAGTTCTCTGAATATGATGGTTTATCTGGC
AAGGTTTGATTCCCTCAAGGAAGTTCCAGGCTAAAAGAGGAGCTAAGCTTCTACAGTCTCTGAGCTTTTTGT
GTTACTGATCTTGAGTCTTATTAAAAAAAAAAAAAAAAAAAAAAAAACCCTGACTTTATCTGGCCGTGCCAG
GCTCCCTTCTGGGCCCTTGCTGCCGTGTGTCAGTACGCTGTAACTAGAGATGGATTATGTAAGAAATGTTTT
TGTCAGAAGAGGCTGTTGCAGTATTTTATGTGCCCTGGTGCACAGCACCTCTGCTGTGAGTCCCCTCCCCGT
GTGAGTTGCAGCCTCGCTGGATACACCTGCTACTGGTCTCAAGCACCTGTGATTTATTGGTCATGAAGCAGG
TACTGGGACTCCTGCTTTTATTATCTGCACGGCCATCCCTCAGCCAGTATAGATGCCCAGGCTAGACTTGCA
GAGCATGAAGAAGGAAGCAGAACCAGTTTGGGACCTTCGCCTGAGCAGCTGACTCAACTCCACTGCCAAGCA
GCCAGGAATCTGGCACAGTTTCTGAGATGTCTCAGCCAACTGGTATTTCTGAAGCCATAGTTTTCCTCTGAG
CTCCCCCTCAACATGGGTATAGTCATTGTGCTTGTTTTGCTTCTTCCTTGTTCTTGTTTAAAAGCCAGTTTT
CTTCCTTGAAAGGCAGTGAAATCTCTGGGTTTTCCATGTGGGTAGAGAGGAGCAGGCGGAACAAGCTTAGGG
AGGCCAGGCGGTAACAGGTAACCATTTCTTGAAGTTGATCCTCAGAGCATCCAAGCCAGTGGGTTCATTTGG
ATGATCTTCAGCCAGGCATAGTCCCTGGTGCCAAGCCTCAGGATATCAAAGAACTACTTACAAAATATGTGT
TCCTTCATAAGGAAAAGAATGGTTTAGCTCAGAGGGCGTGCCTGCCAATAAATCTCACATAGGTTAAGACAC
AAACTGAAAAAATGCTCAAGGACCATGAGCCACAGTCACTGGAGAAGCCACAGTCATTCATTCTCCAGCAGT
TCCCCTTAACCACCTACCA

In some embodiments, the MRC1 enhancer comprises or consists of a nucleotide sequence which is at least 70% identical to any one of SEQ ID NOs: 17-30 or a fragment thereof. Suitably, the MRC1 enhancer comprises or consists of a nucleotide sequence which is at least 80%, at least 85%, at least 90%, at least 95%, at least 98%, or at least 99% identical to any one of SEQ ID NOs: 17-30 or a fragment thereof.

In some embodiments of the invention, the MRC1 enhancer comprises or consists of a nucleotide sequence which is at least 70% identical to SEQ ID NO: 32 or a fragment thereof. Suitably, the MRC1 enhancer comprises or consists of a nucleotide sequence which is at least 80%, at least 85%, at least 90%, at least 95%, at least 98%, or at least 99% identical to SEQ ID NO: 32 or a fragment thereof.

In some embodiments of the invention, the MRC1 enhancer comprises or consists of the nucleotide sequence SEQ ID NO: 32 or a fragment thereof.

Exemplary XhoI-Human.MRC1.enhancer
(SEQ ID NO: 32)
CTCGAGAGCCCCACCATGTTATTGATGGCCAAACAATACGCATGCTGACA
GCCATTATCTGTGGCCTCTGATGCTATTAGCCAAACCATGTTATTGATGG
TCAAACAATACGCATGCTGACAGCCATTATCTGGGACTCAGAAAGTTCTG
CATATTCAAGTCAGGCCAGAGGATCCGAGTTCTAATGTTAAGAGAAACCA
ACACACCAACAAGCAAATAAACAAACCTACCCTTGAACCAAAATATACAT
CAATACCTCCGTTGCAAATGGATAAATGGAACTGCATTGCCCTCTGCTGT
TGGGGAATCTTGGCAACCATTTCAACTCTATGGCTGGAGATGACTTACTG
CTCTGTTTATTTTCCATCCTCCTGCTTAGATTATTGCTTTCAAAGTTTCC
AGAATAGAAGAAGTCAGTGGTGGCCAGTTGTCCTTTAATGGTCTCTTATC
TACCAATGGCTAGTATCCTTTTTGCATTATCGTAGCTCTACTCTTGTAGA
TGTTAAATT

miRNA Target Sequence

The vector of the present invention may comprise one or more miRNA target sequences. Suitably, the transgene is operably linked to one or more miRNA target sequences.

MicroRNA (miRNA) genes are scattered across all human chromosomes, except for the Y chromosome. They can be either located in non-coding regions of the genome or within introns of protein-coding genes. Around 50% of miRNAs appear in clusters which are transcribed as polycistronic primary transcripts. Similar to protein-coding genes, miRNAs are usually transcribed from polymerase-II promoters, generating a so-called primary miRNA transcript (pri-miRNA). This pri-miRNA is then processed through a series of endonucleolytic cleavage steps, performed by two enzymes belonging to the RNAse Type III family, Drosha and Dicer. From the pri-miRNA, a stem loop of about 60 nucleotides in length, called miRNA precursor (pre-miRNA), is excised by a specific nuclear complex, composed of Drosha and DiGeorge syndrome critical region gene (DGCR8), which crops both strands near the base of the primary stem loop and leaves a 5′ phosphate and a 2 bp long, 3′ overhang. The pre-miRNA is then actively transported from the nucleus to the cytoplasm by RAN-GTP and Exportin. Then, Dicer performs a double strand cut at the end of the stem loop not defined by the Drosha cut, generating a 19-24 bp duplex, which is composed of the mature miRNA and the opposite strand of the duplex, called miRNA*. In agreement with the thermodynamic asymmetry rule, only one strand of the duplex is selectively loaded into the RNA-induced silencing complex (RISC), and accumulates as the mature microRNA. This strand is usually the one whose 5′ end is less tightly paired to its complement, as was demonstrated by single-nucleotide mismatches introduced into the 5′ end of each strand of siRNA duplexes. However, there are some miRNAs that support accumulation of both duplex strands to similar extent.

MicroRNAs trigger RNAi, very much like small interfering RNAs (siRNA) which are extensively used for experimental gene knockdown. The main difference between miRNA and siRNA is their biogenesis. Once loaded into RISC, the guide strand of the small RNA molecule interacts with mRNA target sequences preferentially found in the 3′ untranslated region (3′UTR) of protein-coding genes. It has been shown that nucleotides 2-8 counted from the 5′ end of the miRNA, the so-called seed sequence, are essential for triggering RNAi. If the whole guide strand sequence is perfectly complementary to the mRNA target, as is usually the case for siRNAs and plant miRNAs, the mRNA is endonucleolytically cleaved by involvement of the Argonaute (Ago) protein, also called “slicer” of the small RNA duplex into the RNA-induced silencing complex (RISC). DGRC (DiGeorge syndrome critical region gene 8) and TRBP (TAR (HIV) RNA binding protein 2) are double-stranded RNA-binding proteins that facilitate mature miRNA biogenesis by Drosha and Dicer RNase Ill enzymes, respectively. The guide strand of the miRNA duplex gets incorporated into the effector complex RISC, which recognises specific targets through imperfect base-pairing and induces post-transcriptional gene silencing. Several mechanisms have been proposed for this mode of regulation: miRNAs can induce the repression of translation initiation, mark target mRNAs for degradation by deadenylation, or sequester targets into the cytoplasmic P-body.

On the other hand, if only the seed is perfectly complementary to the target mRNA but the remaining bases show incomplete pairing, RNAi acts through multiple mechanisms leading to translational repression. Eukaryotic mRNA degradation mainly occurs through the shortening of the polyA tail at the 3′ end of the mRNA, and de-capping at the 5′ end, followed by 5′-3′ exonuclease digestion and accumulation of the miRNA in discrete cytoplasmic areas, the so called P-bodies, enriched in components of the mRNA decay pathway.

Expression of the transgene may be regulated by one or more endogenous miRNAs using one or more corresponding miRNA target sequences. Using this method, one or more miRNAs endogenously expressed in a cell prevent or reduce transgene expression in that cell by binding to its corresponding miRNA target sequence positioned in the vector or polynucleotide (Brown, B. D. et al. (2007) Nat Biotechnol 25: 1457-1467).

Suitable miRNA target sequences which suppress transgene expression in specific cells will be known to the skilled person. Any suitable method can be used to identify suitable miRNA target sequences, for example by performing microarrays containing known miRNAs, for example from miRbase.

More than one copy of a miRNA target sequence included in the vector may increase the effectiveness of the system. Also it is envisaged that different miRNA target sequences could be included. For example, the transgene may be operably linked to more than one miRNA target sequence, which may or may not be different. The miRNA target sequences may be in tandem, but other arrangements are envisaged. The vector may, for example, comprise 1, 2, 3, 4, 5, 6, 7 or 8 copies of the same or different miRNA target sequence. Suitably, the vector comprises 4 miRNA target sequences of each miRNA target sequence.

The target sequence may be fully or partially complementary to the miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA.

Copies of miRNA target sequences may be separated by a spacer sequence. The spacer sequence may comprise, for example, at least one, at least two, at least three, at least four or at least five nucleotide bases.

The present inventors have found that a vector driving transgene expression from a M2-like macrophage-specific promoter (e.g. the MRC1 promoter) can be used to drive selective transgene expression in Kupffer cells (KCs), and to a lesser extent in MRC1+ splenic macrophages and liver sinusoidal endothelial cells (LSECs). miRNA target sequences can be used to further increase the specificity of the vector.

The one or more miRNA target sequences may suppress transgene expression in some liver cell populations and/or some spleen cell populations. The one or more miRNA target sequences may suppress transgene expression in some liver macrophages and/or some spleen macrophages. For example, expression may be targeted to LSECs.

The term “suppress expression” as used herein may refer to a reduction of expression in the relevant cell type(s) of a transgene to which the one or more miRNA target sequence is operably linked as compared to transgene expression in the absence of the one or more miRNA target sequence, but under otherwise substantially identical conditions. In some embodiments, transgene expression is suppressed by at least 50%. In some embodiments, transgene expression is suppressed by at least 60%, 70%, 80%, 90% or 95%. In some embodiments, transgene expression is substantially prevented.

Suitably, the one or more miRNA target sequence suppresses transgene expression in liver sinusoidal endothelial cells (LSECs) and/or hepatocytes.

In some embodiments, the one or more miRNA target sequence suppresses transgene expression in hepatocytes and/or LSECS. For example, the vector may comprise (i) one or more copies of a miRNA target sequence that suppresses transgene expression in LSECs; and/or (ii) one or more copies of a miRNA target sequence that suppresses transgene expression in hepatocytes.

Suitably, the one or more miRNA target sequence comprises: (i) one or more (e.g. 1, 2, 3, 4, 5, 6, 7, 8) miR-126 target sequence; and/or (ii) one or more (e.g. 1, 2, 3, 4, 5, 6, 7, 8) miR-122 target sequence.

The miR-126 target sequence is an exemplary miRNA target sequence that suppresses transgene expression in LSECs. miR-126 is a microRNA that is expressed in endothelial cells (e.g. LSEC), and when it binds to its target sequence it reduces the expression of the target gene.

In some embodiments of the invention, the miR-126 target sequence comprises or consists of a nucleotide sequence which is at least 70% identical to SEQ ID NO: 3 or a fragment thereof. Suitably, the miR-126 target sequence comprises or consists of a nucleotide sequence which is at least 80%, at least 90% or at least 95% identical to SEQ ID NO: 3 or a fragment thereof.

In some embodiments of the invention, the miR-126 target sequence comprises or consists of the nucleotide sequence SEQ ID NO: 3 or a fragment thereof.

Exemplary miRT-126
(SEQ ID NO: 3)
CGCATTATTACTCACGGTACGA

The miR-122 target sequence is an exemplary miRNA target sequence that suppresses transgene expression in hepatocytes. miR-122 is the most abundant microRNA in hepatocytes, and when it binds to its target sequence it reduces the expression of the target gene.

In some embodiments of the invention, the miR-122 target sequence comprises or consists of a nucleotide sequence which is at least 70% identical to SEQ ID NO: 4 or a fragment thereof.

Suitably, the miR-122 target sequence comprises or consists of a nucleotide sequence which is at least 80%, at least 90% or at least 95% identical to SEQ ID NO: 4 or a fragment thereof.

In some embodiments of the invention, the miR-122 target sequence comprises or consists of the nucleotide sequence SEQ ID NO: 4 or a fragment thereof.

Exemplary miRT-122
(SEQ ID NO: 4)
ACAAACACCATTGTCACACTCCA

Further miRNA target sequences that suppresses transgene expression in LSECs and/or hepatocytes can be identified by any suitable method, for example miRNA expression analysis as described in Oda, S., et al., 2018. The American journal of pathology, 188(4), pp. 916-928.

In some embodiments, the one or more miRNA target sequence comprises: (i) two or more miR-126 target sequences; and/or (ii) two or more miR-122 target sequences. In some embodiments, the one or more miRNA target sequence comprises: (i) four miR-126 target sequences; and/or (ii) four miR-122 target sequences. Suitably, the target sequences are separated by spacer sequences.

In some embodiments of the invention, the one or more miRNA target sequence comprises or consists of a nucleotide sequence which is at least 70% identical to one or more of SEQ ID NOs: 5-7 or a fragment thereof. Suitably, the one or more miRNA target sequence comprises or consists of a nucleotide sequence which is at least 80%, at least 90% or at least 95% identical to one or more of SEQ ID NOs: 5-7 or a fragment thereof.

In some embodiments of the invention, the one or more miRNA target sequence comprises or consists of the nucleotide sequence of one or more of SEQ ID NOs: 5-7 or a fragment thereof.

Exemplary miRT-122 4 x miRT
(SEQ ID NO: 5)
TCTAGATAAACAAACACCATTGTCACACTCCATTCGAAACAAACACCATT
GTCACACTCCAACGCGTACAAACACCATTGTCACACTCCAATGCATACAA
ACACCATTGTCACACTCCACCCGGGTCGAGCTCGGTACC
Exemplary miRT-126 4 x miRT
(SEQ ID NO: 6)
GGTACCAGCAAACGCATTATTACTCACGGTACGACCATCGCATTATTACT
CACGGTACGAACTTCGCATTATTACTCACGGTACGACGAACGCATTATTA
CTCACGGTACGACACGTGTCGGTACC
Exemplary miRT-122 and miR126 4 x miRT
(SEQ ID NO: 7)
GGTACCAGCGCTACAAACACCATTGTCACACTCCAACATACAAACACCAT
TGTCACACTCCAGATTACAAACACCATTGTCACACTCCACAGAACAAACA
CCATTGTCACACTCCAGTTTAAACGCATTATTACTCACGGTACGACCATC
GCATTATTACTCACGGTACGAACTTCGCATTATTACTCACGGTACGACGA
ACGCATTATTACTCACGGTACGACACGTGTCGGTACC

In some embodiments of the invention, the one or more miRNA target sequence comprises or consists of a nucleotide sequence which is at least 70% identical to SEQ ID NO: 36 or a fragment thereof. Suitably, the one or more miRNA target sequence comprises or consists of a nucleotide sequence which is at least 80%, at least 85%, at least 90%, at least 95%, at least 98%, or at least 99% identical to SEQ ID NO: 36 or a fragment thereof.

In some embodiments of the invention, the one or more miRNA target sequence comprises or consists of the nucleotide sequence SEQ ID NO: 36 or a fragment thereof.

Exemplary AfeI-4xmiRT122-4xmiRT126-PmII
(SEQ ID NO: 36)
AGCGCTACAAACACCATTGTCACACTCCAACATACAAACACCATTGTCAC
ACTCCAGATTACAAACACCATTGTCACACTCCACAGAACAAACACCATTG
TCACACTCCAGTTTAAACGCATTATTACTCACGGTACGACCATCGCATTA
TTACTCACGGTACGAACTTCGCATTATTACTCACGGTACGACGAACGCAT
TATTACTCACGGTACGACACGTGTC

In some embodiments, the one or more miRNA target sequence suppresses transgene expression in some liver and/or some splenic macrophages. For example, the one or more miRNA target sequence may suppress transgene expression in M2-like macrophages. For example, the one or more miRNA target sequence may suppress transgene expression in Kupffer cells and/or MRC1+ splenic macrophages.

In some embodiments, the one or more miRNA target sequence suppresses transgene expression in splenic phagocytes (e.g., splenic macrophages).

miRNA target sequences that suppresses transgene expression in some liver and/or some splenic macrophages can be identified by any suitable method, for example miRNA expression analysis as described in Zhang, Y., et al., 2013. International journal of molecular medicine, 31(4), pp. 797-802.

### Other Expression Control Sequences

The vector of the present invention may further comprise one or more regulatory elements which may act pre- or post-transcriptionally. Suitably, the transgene is operably linked to one or more regulatory elements which may act pre- or post-transcriptionally. The one or more regulatory elements may facilitate expression of the transgene in phagocytes.

A “regulatory element” is any nucleotide sequence which facilitates expression of a polypeptide, e.g. acts to increase expression of a transcript or to enhance mRNA stability. Suitable regulatory elements include for example promoters, enhancer elements, post-transcriptional regulatory elements and polyadenylation sites.

### Post-Transcriptional Regulatory Elements

The vector of the present invention may comprise one or more post-transcriptional regulatory elements. Suitably, the transgene is operably linked to one or more post-transcriptional regulatory elements. The post-transcriptional regulatory element may improve gene expression.

The vector of the present invention may comprise a Woodchuck Hepatitis Virus Post-transcriptional Regulatory Element (WPRE). Suitably, the transgene is operably linked to a WPRE.

In some embodiments of the invention, the WPRE comprises or consists of a nucleotide sequence which is at least 70% identical to SEQ ID NO: 35 or a fragment thereof. Suitably, the WPRE comprises or consists of a nucleotide sequence which is at least 80%, at least 85%, at least 90%, at least 95%, at least 98%, or at least 99% identical to SEQ ID NO: 35 or a fragment thereof.

In some embodiments of the invention, the WPRE comprises or consists of the nucleotide sequence SEQ ID NO: 35 or a fragment thereof.

Exemplary SalI-WPRE
(SEQ ID NO: 35)
GTCGACCCGACAGTTTCGACAATCAACCTCTGGATTACAAAATTTGTGAA
AGATTGACTGGTATTCTTAACTATGTTGCTCCTTTTACGCTATGTGGATA
CGCTGCTTTAATGCCTTTGTATCATGCTATTGCTTCCCGTATGGCTTTCA
TTTTCTCCTCCTTGTATAAATCCTGGTTGCTGTCTCTTTATGAGGAGTTG
TGGCCCGTTGTCAGGCAACGTGGCGTGGTGTGCACTGTGTTTGCTGACGC
AACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGC
CTTGCCCGCTGCTGGACAGGGGCTCGGCTGTTGGGCACTGACAATTCCGT
GGTGTTGTCGGGGAAATCATCGTCCTTTCCTTGGCTGCTCGCCTGTGTTG
CCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCT
TCCGCGTCTTCGCCTTCGCCCTCAGACGAGTCGGATCTCCCTTTGGGCCG
CCTCCCCGCCTGGAATTCGAGCTCGGTACC

### Destabilising Domain

The vector of the present invention may comprise a nucleotide sequence encoding a destabilising domain. Suitably, the transgene is operably linked to a destabilising domain, i.e. in frame with the transgene product, such that when the transgene is translated a fusion protein is produced comprising the destabilising domain fused to the transgene product.

Destabilization domains (DDs) represent a fusion protein component that is intrinsically unstable and destabilizes other proteins upon incorporation, leading to protein degradation. A well-known example of DDs is the Shield system, which incorporated a rampamycin-binding protein (FKBP12) into proteins as a build-in destabilising domain to cause protein degradation in cells. In the absence of its specific ligand (Shield-1), the protein is degraded by the proteasome (Banaszynski, L. A., et al., 2006. Cell, 126(5), pp. 995-1004).

Another exemplary destabilization domains is dihydrofolate reductase (DHFR), or a variant thereof. In mammalian cells, fusion proteins containing the DHFR protein are rapidly ubiquitinated and degraded by the proteasome system. The antibiotic trimethoprim (TMP) or a TMP-based small molecule can bind to the DHFR protein and prevent the protein from being degraded, which allows the fusion protein to escape degradation (Peng, H., et al., 2019. Molecular Therapy-Methods & Clinical Development, 15, pp. 27-39).

The vector of the present invention may comprise a dihydrofolate reductase coding sequence, or a variant or derivative thereof. Suitably, the transgene is operably linked to a dihydrofolate reductase coding sequence (or a variant or derivative thereof), i.e. in frame with the transgene product, such that when the transgene is translated a fusion protein is produced comprising the dihydrofolate reductase coding sequence (or a variant or derivative thereof) fused to the transgene product.

### Polyadenylation Sequence

The vector of the present invention may comprise a polyadenylation sequence. Suitably, the transgene is operably linked to a polyadenylation sequence. A polyadenylation sequence may be inserted after the transgene to improve transgene expression.

A polyadenylation sequence typically comprises a polyadenylation signal, a polyadenylation site and a downstream element: the polyadenylation signal comprises the sequence motif recognised by the RNA cleavage complex; the polyadenylation site is the site of cleavage at which a poly-A tails is added to the mRNA; the downstream element is a GT-rich region which usually lies just downstream of the polyadenylation site, which is important for efficient processing.

### Kozak Sequence

The vector of the present invention may comprise a Kozak sequence. Suitably, the transgene is operably linked to a Kozak sequence. A Kozak sequence may be inserted before the start codon to improve the initiation of translation.

In some embodiments of the invention, the Kozak sequence comprises or consists of a nucleotide sequence which is at least 70% identical to SEQ ID NO: 33 or a fragment thereof. Suitably, the Kozak sequence comprises or consists of a nucleotide sequence which is at least 80%, at least 85%, at least 90%, at least 95%, at least 98%, or at least 99% identical to SEQ ID NO: 33 or a fragment thereof.

In some embodiments of the invention, the Kozak Sequence comprises or consists of the nucleotide sequence SEQ ID NO: 33 or a fragment thereof.

Exemplary BamHI-KOZAC
(SEQ ID NO: 33)
GGATCCGCCACC

### Transgene

The vector of the present invention may comprise one or more transgenes. Suitably, the one or more expression control sequence is operably linked to a transgene.

The transgene is not particularly limited and any suitable transgene may be used.

The transgene may encode a naturally-occurring human gene, or a variant and/or fragment thereof.

The transgene may be a therapeutic transgene.

The transgene may encode a therapeutic polypeptide and/or an antigenic polypeptide.

In some embodiments, the transgene comprises a nucleotide sequence encoding a signal peptide, preferably wherein the signal peptide is operably linked to the encoded polypeptide (e.g. therapeutic polypeptide and/or antigenic polypeptide). The signal peptide may, for example, be a natural signal peptide of the encoded polypeptide. In some embodiments, the transgene does not comprise a nucleotide sequence encoding a signal peptide.

### Therapeutic Polypeptides

Suitably, the transgene encodes a therapeutic polypeptide.

As used herein, a “therapeutic polypeptide” is any polypeptide which can be used for therapy. For example, therapeutic polypeptides include therapeutic cytokines that can activate immune responses.

In some embodiments, the transgene encodes a cytokine, for example a cytokine that can activate immune responses, particularly anti-tumour responses.

Cytokines are molecular messengers that allow the cells of the immune system to communicate with one another to generate a coordinated, robust, but self-limited response to a target antigen. Cytokines directly stimulate immune effector cells and stromal cells at the tumour site, enhance tumour cell recognition by cytotoxic effector cells. Cytokines may have broad anti-tumour activity (Lee, S. and Margolin, K., 2011. Cancers, 3(4), pp. 3856-3893).

For example, any cytokine which can activate immune responses, particularly anti-tumour responses can be used. Exemplary cytokines include IFNα, IFNβ, IFNγ, IL-2, IL-12, TNFα, CXCL9, and IL-1β. Further exemplary cytokines include IL10, IL15 or IL18.

A variant of any of these cytokines may be used, provided that the variant retains the capacity to activate immune responses, particularly anti-tumour responses. A skilled person will be arrive at such variants using methods known in the art. The variant may have at least 70%, at least 80%, at least 90%, at least 95%, or at least 99% identity to any of the cytokines.

A fragment of any of these cytokines (or variants thereof) may be used, provided that the fragment retains the capacity to activate immune responses, particularly anti-tumour responses. A skilled person will be able to arrive at such fragments using methods known in the art. For example, a fragment may retain residues or domains necessary to activate an immune response.

In some embodiments, the transgene encodes a cytokine selected from IFNα, IFNβ, IFNγ, IL-2, IL-12, TNFα, CXCL9, and IL-1β, or variants and/or fragments thereof. In some embodiments, the transgene encodes a cytokine selected from IL10, IL15 or IL18, or variants and/or fragments thereof.

Interferons There are three major types of interferon (IFN). The human type I IFN genes encode a family of 17 distinct proteins (including 13 sub-types of IFNα, plus IFNβ, IFNε, IFNκ and IFNω). There is only a single type II IFN, IFNγ. The type Ill IFNs consist of IFNλ1, IFNλ2, IFNλ3, and IFNλ4. All IFNs have the potential to act on tumour cells to exert direct anti-tumour effects or on immune cells, exerting indirect anti-tumour effects (Parker, B. S., et al., 2016. Nature Reviews Cancer, 16(3), p. 131).

In some embodiments, the transgene encodes an interferon, for example a Type I interferon (e.g. IFNα, IFNβ), a Type II interferon (e.g. IFNγ), or a Type III interferon (e.g. IFNλ, IFNλ2, IFNλ3, IFNλ4). In some embodiments, the transgene encodes a Type I interferon (e.g. IFNα, IFNβ).

### IFNα

Interferon-alpha (IFNα), a type 1 interferon, is a pleiotropic cytokine playing key role in defending the organism against viral infections. It is well established that IFNα can exert anti-tumour functions including direct tumour cell killing, activation of adaptive and innate immune functions and angiostatic activity. IFNα has been approved for clinical use for several types of tumours, including melanoma, renal cell carcinoma and Kaposi's sarcoma. However, recombinant IFNα alone is not well tolerated when administered systemically, thus alternative therapeutic options to IFNα are currently preferred.

The vector of the present invention may reduce the systemic toxic effects associated to IFNα delivery by delivering therapeutic IFNα selectively to tumours. Routes of administration and expected target cells as phagocytic cells whose physiological turnover may facilitate natural loss of the vector.

In some embodiments, the transgene encodes IFNα. An exemplary human interferon-alpha (IFNα) for use in the present invention is UniProtKB P01562.

In some embodiments of the invention, the transgene encodes a polypeptide which comprises or consists of an amino acid sequence which is at least 70% identical to SEQ ID NO: 8 or a fragment thereof. Suitably, the transgene encodes a polypeptide which comprises or consists of an amino acid sequence which is at least 80%, at least 90% or at least 95% identical to SEQ ID NO: 8 or a fragment thereof.

In some embodiments of the invention, the transgene encodes a polypeptide which comprises or consists of the amino acid sequence SEQ ID NO: 8 or a fragment thereof.

Exemplary human interferon-alpha
(SEQ ID NO: 8)
MASPFALLMVLVVLSCKSSCSLGCDLPETHSLDNRRTLMLLAQMSRISPS
SCLMDRHDFGFPQEEFDGNQFQKAPAISVLHELIQQIFNLFTTKDSSAAW
DEDLLDKFCTELYQQLNDLEACVMQEERVGETPLMNADSILAVKKYFRRI
TLYLTEKKYSPCAWEVVRAEIMRSLSLSTNLQERLRRKE

In some embodiments of the invention, the transgene comprises or consists of a nucleotide sequence which is at least 70% identical to SEQ ID NO: 34 or a fragment thereof. Suitably, the transgene comprises or consists of a nucleotide sequence which is at least 80%, at least 85%, at least 90%, at least 95%, at least 98%, or at least 99% identical to SEQ ID NO: 34 or a fragment thereof.

In some embodiments of the invention, the transgene comprises or consists of the nucleotide sequence SEQ ID NO: 34 or a fragment thereof.

Exemplary Human.IFNA
(SEQ ID NO: 34)
ATGGCCTCGCCCTTTGCTTTACTGATGGTCCTGGTGGTGCTCAGCTGCAA
GTCAAGCTGCTCTCTGGGCTGTGATCTCCCTGAGACCCACAGCCTGGATA
ACAGGAGGACCTTGATGCTCCTGGCACAAATGAGCAGAATCTCTCCTTCC
TCCTGTCTGATGGACAGACATGACTTTGGATTTCCCCAGGAGGAGTTTGA
TGGCAACCAGTTCCAGAAGGCTCCAGCCATCTCTGTCCTCCATGAGCTGA
TCCAGCAGATCTTCAACCTCTTTACCACAAAAGATTCATCTGCTGCTTGG
GATGAGGACCTCCTAGACAAATTCTGCACCGAACTCTACCAGCAGCTGAA
TGACTTGGAAGCCTGTGTGATGCAGGAGGAGAGGGTGGGAGAAACTCCCC
TGATGAATGCGGACTCCATCTTGGCTGTGAAGAAATACTTCCGAAGAATC
ACTCTCTATCTGACAGAGAAGAAATACAGCCCTTGTGCCTGGGAGGTTGT
CAGAGCAGAAATCATGAGATCCCTCTCTTTATCAACAAACTTGCAAGAAA
GATTAAGGAGGAAGGAATAA

### IFNβ

In some embodiments, the transgene encodes IFNβ. An exemplary human IFNβ for use in the present invention is UniProtKB P01574.

In some embodiments of the invention, the transgene encodes a polypeptide which comprises or consists of an amino acid sequence which is at least 70% identical to SEQ ID NO: 9 or a fragment thereof. Suitably, the transgene encodes a polypeptide which comprises or consists of an amino acid sequence which is at least 80%, at least 90% or at least 95% identical to SEQ ID NO: 9 or a fragment thereof.

In some embodiments of the invention, the transgene encodes a polypeptide which comprises or consists of the polypeptide sequence SEQ ID NO: 9 or a fragment thereof.

Exemplary human interferon-beta
(SEQ ID NO: 9)
MTNKCLLQIALLLCFSTTALSMSYNLLGFLQRSSNFQCQKLLWQLNGRLE
YCLKDRMNFDIPEEIKQLQQFQKEDAALTIYEMLQNIFAIFRQDSSSTGW
NETIVENLLANVYHQINHLKTVLEEKLEKEDFTRGKLMSSLHLKRYYGRI
LHYLKAKEYSHCAWTIVRVEILRNFYFINRLIGYLRN

### IFNγ

In some embodiments, the transgene encodes IFNγ. An exemplary human IFNγ for use in the present invention is UniProtKB P01579.

In some embodiments of the invention, the transgene encodes a polypeptide which comprises or consists of an amino acid sequence which is at least 70% identical to SEQ ID NO: 10 or a fragment thereof. Suitably, the transgene encodes a polypeptide which comprises or consists of an amino acid sequence which is at least 80%, at least 90% or at least 95% identical to SEQ ID NO: 10 or a fragment thereof.

In some embodiments of the invention, the transgene encodes a polypeptide which comprises or consists of the polypeptide sequence SEQ ID NO: 10 or a fragment thereof.

Exemplary human interferon-gamma
(SEQ ID NO: 10)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ

### Other Cytokines

### IL-2

Interleukin-2 (IL-2), as well as other members of the IL-2-related family of T cell growth factors (e.g., IL-4, IL-7, IL-9, IL-15, and IL-21), utilize a common receptor signalling system that results in the activation and expansion of CD4+ and CD8+ T cells (Lee, S. and Margolin, K., 2011. Cancers, 3(4), pp. 3856-3893).

In some embodiments, the transgene encodes IL-2 or an IL-2 related-cytokine (e.g. IL-7, IL-15, IL-21). An exemplary human IL-2 for use in the present invention is UniProtKB P60568.

In some embodiments of the invention, the transgene encodes a polypeptide which comprises or consists of an amino acid sequence which is at least 70% identical to SEQ ID NO: 11 or a fragment thereof. Suitably, the transgene encodes a polypeptide which comprises or consists of an amino acid sequence which is at least 80%, at least 90% or at least 95% identical to SEQ ID NO: 11 or a fragment thereof.

In some embodiments of the invention, the transgene encodes a polypeptide which comprises or consists of the polypeptide sequence SEQ ID NO: 11 or a fragment thereof.

Exemplary human Interleukin-2 (SEQ ID NO: 11)
(SEQ ID NO: 11)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

### IL-12

In some embodiments, the transgene encodes IL-12. Exemplary human IL-12 alpha and beta subunits for use in the present invention are UniProtKBs P29459 and P29460.

In some embodiments of the invention, the transgene encodes a polypeptide which comprises or consists of an amino acid sequence which is at least 70% identical to SEQ ID NO: 12 or a fragment thereof. Suitably, the transgene encodes a polypeptide which comprises or consists of an amino acid sequence which is at least 80%, at least 90% or at least 95% identical to SEQ ID NO: 12 or a fragment thereof.

In some embodiments of the invention, the transgene encodes a polypeptide which comprises or consists of the polypeptide sequence SEQ ID NO: 12 or a fragment thereof.

Exemplary human Interleukin-12 subunit alpha
(SEQ ID NO: 12)
MCPARSLLLVATLVLLDHLSLARNLPVATPDPGMFPCLHHSQNLLRAVSN
MLQKARQTLEFYPCTSEEIDHEDITKDKISTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS

In some embodiments of the invention, the transgene encodes a polypeptide which comprises or consists of an amino acid sequence which is at least 70% identical to SEQ ID NO: 13 or a fragment thereof. Suitably, the transgene encodes a polypeptide which comprises or consists of an amino acid sequence which is at least 80%, at least 90% or at least 95% identical to SEQ ID NO: 13 or a fragment thereof.

In some embodiments of the invention, the transgene encodes a polypeptide which comprises or consists of the polypeptide sequence SEQ ID NO: 13 or a fragment thereof.

Exemplary human Interleukin-12 subunit beta
(SEQ ID NO: 13)
MCHQQLVISWFSLVFLASPLVAIWELKKDVYVVELDWYPDAPGEM
VVLTCDTPEEDGITWILDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCS

In some embodiments, the transgene encodes a single chain IL112. The single chain IL12 may comprise IL12 subunit beta (e.g. the amino acid sequence SEQ ID NO: 13 or a fragment thereof, or a sequence that is at least 70%, at least 80%, at least 90% or at least 95% identical to SEQ ID NO: 13 or a fragment thereof) and IL12 subunit alpha (the amino acid sequence SEQ ID NO: 12 or a fragment thereof, or a sequence that is at least 70%, at least 80%, at least 90% or at least 95% identical to SEQ ID NO: 12 or a fragment thereof). The single chain IL12 may be a fusion protein comprising the IL12 subunit beta and the IL12 subunit alpha. The IL12 subunit beta and IL12 subunit alpha may be joined by a linker sequence. The linker sequence may comprise or consist of the amino acid sequence SEQ ID NO: 42 or a fragment thereof, or a sequence that is at least 70%, at least 80%, at least 90% or at least 95% identical to SEQ ID NO: 42 or a fragment thereof.

In some embodiments, the transgene encodes a polypeptide which comprises or consists of an amino acid sequence which is at least 70% identical to SEQ ID NO: 37 or 46 or a fragment thereof. Suitably, the transgene encodes a polypeptide which comprises or consists of an amino acid sequence which is at least 80%, at least 90% or at least 95% identical to SEQ ID NO: 37 or 46 or a fragment thereof.

In some embodiments of the invention, the transgene encodes a polypeptide which comprises or consists of the polypeptide sequence SEQ ID NO: 37 or 46 or a fragment thereof.

Exemplary single chain human Interleukin-12
sequences:
(SEQ ID NO: 37)
MCPQKLTISWFAIVLLVSPLMAIAGQLMWELKKDVYVVELDWYPD
APGEMVVLTCDTPEEDGITWTLDQSSEVLGSGKTLTIQVKEFGDA
GQYTCHKGGEVLSHSLLLLHKKEDGIWSTDILKDQKEPKNKTFLR
CEAKNYSGRFTCWWLTTISTDLTFSVKSSRGSSDPQGVTCGAATL
SAERVRGDNKEYEYSVECQEDSACPAAEESLPIEVMVDAVHKLKY
ENYTSSFFIRDIIKPDPPKNLQLKPLKNSRQVEVSWEYPDTWSTP
HSYFSLTFCVQVQGKSKREKKDRVFTDKTSATVICRKNASISVRA
QDRYYSSSWSEWASVPCSRAGGGGSGGGGSGGGGSRIRNLPVATP
DPGMFPCLHHSQNLLRAVSNMLQKARQTLEFYPCTSEEIDHEDIT
KDKTSTVEACLPLELTKNESCLNSRETSFITNGSCLASRKTSFMM
ALCLSSIYEDLKMYQVEFKTMNAKLLMDPKRQIFLDQNMLAVIDE
LMQALNENSETVPQKSSLEEPDFYKTKIKLCILLHAFRIRAVTID
RVMSYLNAS
{SEQ ID NO: 46}
WELKKDVYVVELDWYPDAPGEMVVLTCDTPEEDGITWILDQSSEV
LGSGKTLTIQVKEFGDAGQYTCHKGGEVLSHSLLLLHKKEDGIWS
TDILKDQKEPKNKTFLRCEAKNYSGRFTCWWLTTISTDLTFSVKS
SRGSSDPQGVTCGAATLSAERVRGDNKEYEYSVECQEDSACPAAE
ESLPIEVMVDAVHKLKYENYTSSFFIRDIIKPDPPKNLQLKPLKN
SRQVEVSWEYPDTWSTPHSYFSLTFCVQVQGKSKREKKDRVFTDK
TSATVICRKNASISVRAQDRYYSSSWSEWASVPCSRAGGGGSGGG
GSGGGGSRTRNLPVATPDPGMFPCLHHSQNLLRAVSNMLQKARQT
LEFYPCTSEEIDHEDITKDKTSTVEACLPLELTKNESCLNSRETS
FITNGSCLASRKTSFMMALCLSSIYEDLKMYQVEFKTMNAKLLMD
PKRQIFLDQNMLAVIDELMQALNFNSETVPQKSSLEEPDFYKTKI
KLCILLHAFRIRAVTIDRVMSYLNAS

In some embodiments of the invention, the transgene comprises or consists of a nucleotide sequence which is at least 70% identical to SEQ ID NO: 40 or a fragment thereof. Suitably, the transgene comprises or consists of a nucleotide sequence which is at least 80%, at least 85%, at least 90%, at least 95%, at least 98%, or at least 99% identical to SEQ ID NO: 40 or a fragment thereof.

In some embodiments of the invention, the transgene comprises or consists of the nucleotide sequence SEQ ID NO: 40 or a fragment thereof.

### IL10

In some embodiments, the transgene encodes IL-10.

In some embodiments of the invention, the transgene encodes a polypeptide which comprises or consists of an amino acid sequence which is at least 70% identical to SEQ ID NO: 38 or a fragment thereof. Suitably, the transgene encodes a polypeptide which comprises or consists of an amino acid sequence which is at least 80%, at least 90% or at least 95% identical to SEQ ID NO: 38 or a fragment thereof.

In some embodiments of the invention, the transgene encodes a polypeptide which comprises or consists of the polypeptide sequence SEQ ID NO: 38 or a fragment thereof.

Exemplary human Interleukin-10
(SEQ ID NO: 38)
MHSSALLCCLVLLTGVRASPGQGTQSENSCTHFPGNLPNMLRDLR
DAFSRVKTFFQMKDQLDNLLLKESLLEDFKGYLGCQALSEMIQFY
LEEVMPQAENQDPDIKAHVNSLGENLKTLRLRLRRCHRFLPCENK
SKAVEQVKNAFNKLQEKGIYKAMSEFDIFINYIEAYMTMKIRN

In some embodiments of the invention, the transgene comprises or consists of a nucleotide sequence which is at least 70% identical to SEQ ID NO: 39 or a fragment thereof. Suitably, the transgene comprises or consists of a nucleotide sequence which is at least 80%, at least 85%, at least 90%, at least 95%, at least 98%, or at least 99% identical to SEQ ID NO: 39 or a fragment thereof.

In some embodiments of the invention, the transgene comprises or consists of the nucleotide sequence SEQ ID NO: 39 or a fragment thereof.

### IL15

In some embodiments, the transgene encodes IL-15.

In some embodiments of the invention, the transgene encodes a polypeptide which comprises or consists of an amino acid sequence which is at least 70% identical to SEQ ID NO: 44 or a fragment thereof. Suitably, the transgene encodes a polypeptide which comprises or consists of an amino acid sequence which is at least 80%, at least 90% or at least 95% identical to SEQ ID NO: 44 or a fragment thereof.

In some embodiments of the invention, the transgene encodes a polypeptide which comprises or consists of the polypeptide sequence SEQ ID NO: 44 or a fragment thereof.

Exemplary human Interleukin-15
(SEQ ID NO: 44)
NWVNVISDLKKIEDLIQSMHIDATLYTESDVHPSCKVTAMKCFLL
ELQVISLESGDASIHDTVENLIILANNSLSSNGNVTESGCKECEE
LEEKNIKEFLQSFVHIVQMFINTS

### IL18

In some embodiments, the transgene encodes IL-18.

In some embodiments of the invention, the transgene encodes a polypeptide which comprises or consists of an amino acid sequence which is at least 70% identical to SEQ ID NO: 45 or 47 or a fragment thereof. Suitably, the transgene encodes a polypeptide which comprises or consists of an amino acid sequence which is at least 80%, at least 90% or at least 95% identical to SEQ ID NO: 45 or 47 or a fragment thereof.

In some embodiments of the invention, the transgene encodes a polypeptide which comprises or consists of the polypeptide sequence SEQ ID NO: 45 or 47 or a fragment thereof.

(SEQ ID NO: 45)
Exemplary human Interleukin-18 sequences:
YFGKLESKLSVIRNLNDQVLFIDQGNRPLFEDMTDSDCRDNAPRT
IFIISMYKDSQPRGMAVTISVKCEKISTLSCENKIISFKEMNPPD
NIKDIKSDIIFFQRSVPGHDNKMQFESSSYEGYFLACEKERDLFK
LILKKEDELGDRSIMFTVQNED
(SEQ ID NO: 47)
YFGKLESKLSVIRNLNDQVLFIDQGNRPLFEDMTDSDCRDNAPRT
IFIISAYGDSRARGKAVTISVKCEKISTLSCENKIISFKEMNPPD
NIKDIKSDIIFFQRSVPGHDNKMQFESSSYEGYFLACEKERDLFK
LILKKEDELGDRSIMFTVQNED

### TNFα

In some embodiments, the transgene encodes Tumour necrosis factor alpha (TNFα). An exemplary human TNFα for use in the present invention is UniProtKB P01375.

In some embodiments of the invention, the transgene encodes a polypeptide which comprises or consists of an amino acid sequence which is at least 70% identical to SEQ ID NO: 14 or a fragment thereof. Suitably, the transgene encodes a polypeptide which comprises or consists of an amino acid sequence which is at least 80%, at least 90% or at least 95% identical to SEQ ID NO: 14 or a fragment thereof.

In some embodiments of the invention, the transgene encodes a polypeptide which comprises or consists of the polypeptide sequence SEQ ID NO: 14 or a fragment thereof.

Exemplary human TNFα
(SEQ ID NO: 14)
MSTESMIRDVELAEEALPKKTGGPQGSRRCLFLSLFSFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

### CXCL9

In some embodiments, the transgene encodes C—X—C motif chemokine 9 (CXCL9).

An exemplary human CXCL9 for use in the present invention is UniProtKB 007325.

In some embodiments of the invention, the transgene encodes a polypeptide which comprises or consists of an amino acid sequence which is at least 70% identical to SEQ ID NO: 15 or a fragment thereof. Suitably, the transgene encodes a polypeptide which comprises or consists of an amino acid sequence which is at least 80%, at least 90% at least 95% identical to SEQ ID NO: 15 or a fragment thereof.

In some embodiments of the invention, the transgene encodes a polypeptide which comprises or consists of the polypeptide sequence SEQ ID NO: 15 or a fragment thereof.

Exemplary human CXCL9
(SEQ ID NO: 15)
MKKSGVLFLLGIILLVLIGVQGTPVVRKGRCSCISTNQGTIHLQS
LKDLKQFAPSPSCEKIEIIATLKNGVQTCLNPDSADVKELIKKWE
KQVSQKKKQKNGKKHQKKKVLKVRKSQRSRQKKTT

### IL-1β

In some embodiments, the transgene encodes interleukin-1 beta (IL-1β). An exemplary human IL-1p for use in the present invention is UniProtKB P01584.

In some embodiments of the invention, the transgene encodes a polypeptide which comprises or consists of an amino acid sequence which is at least 70% identical to SEQ ID NO: 16 or a fragment thereof. Suitably, the transgene encodes a polypeptide which comprises or consists of an amino acid sequence which is at least 80%, at least 90% or at least 95% identical to SEQ ID NO: 16 or a fragment thereof.

In some embodiments of the invention, the transgene encodes a polypeptide which comprises or consists of the polypeptide sequence SEQ ID NO: 16 or a fragment thereof.

Exemplary human IL-1β
(SEQ ID NO: 16)
MAEVPELASEMMAYYSGNEDDLFFEADGPKQMKCSFQDLDLCPLD
GGIQLRISDHHYSKGFRQAASVVVAMDKLRKMLVPCPQTFQENDL
STFFPFIFEEEPIFFDTWDNEAYVHDAPVRSLNCTLRDSQQKSLV
MSGPYELKALHLQGQDMEQQVVFSMSFVQGEESNDKIPVALGLKE
KNLYLSCVLKDDKPTLQLESVDPKNYPKKKMEKRFVFNKIEINNK
LEFESAQFPNWYISTSQAENMPVFLGGTKGGQDITDFTMQFVSS

### Antigenic Polypeptides

Suitably, the transgene encodes an antigenic polypeptide.

As used herein, an “antigenic polypeptide” is any polypeptide which can induce an immune response. In particular, an antigenic polypeptide may be internalized and presented by an antigen-presenting cell (APC). Antigen presentation allows for specificity of adaptive immunity and can contribute to immune responses against both intracellular and extracellular pathogens. APCs also naturally have a role in fighting tumours, via stimulation of B and cytotoxic T cells to respectively produce antibodies against tumour-related antigens and kill malignant cells.

### Tumour Antigen

In some embodiments, the transgene encodes a tumour antigen, for example a tumour-specific antigen or a tumour-associated antigen.

As used herein, a “tumour antigen” is an antigenic substance (e.g. antigenic polypeptide) produced in tumour cells. A “tumour-specific antigen” is present only on tumours cells and not on any other cell. A “tumour-associated antigen” is present on some tumour cells and also some normal cells.

Any suitable tumour antigen can be used. Suitable tumour antigens will be well known to those of skill in the art, for example tumour antigens are recorded in the Cancer Antigenic Peptide Database.

Certain tumours have certain tumours antigens in abundance. Certain tumours antigens are thus used as tumours markers and can also be used in cancer therapy as tumour antigen vaccines.

Similar to vaccines against pathogens, tumour vaccines consist in the delivery of inactivated cancer cells or tumour antigens (TA) in combination with adjuvants. Tumour vaccines also include DCs challenged ex vivo with TAs. Despite several years of experimentation, tumour vaccines have mostly delivered disappointing results, leading to only one tumour vaccine approved for clinical use. Identifying new vaccine delivery systems that bypass the barriers to effective cancer vaccines should enable their therapeutic applicability.

The vector of the present invention may represent a valid strategy to design tumour vaccines.

In some embodiments, the transgene encodes a tumour antigen which is abundant on liver metastases.

In some embodiments, the transgene encodes a tumour antigen selected from carcinoembryonic antigen (CEA), melanoma associated antigen (MAGE) family, cancer germline (CAGE) family, B melanoma antigen (BAGE-1), synovial sarcoma x breakpoint 20 (SSX-2), Sarcoma antigen (SAGE) family, LAGE1, NY-ESO-1, HER2, EGFR, MUC-1, and GAST.

The invention contemplates the combined use of the cytokine gene therapy of the invention and the tumour vaccine of the invention.

In another aspect, the invention provides a product (e.g. a composition or kit) comprising a first vector of the invention comprising a transgene encoding a cytokine (preferably IL10) and a second vector of the invention comprising a transgene encoding a tumour antigen.

In another aspect, the invention provides a product (e.g., a composition or kit) comprising a cell comprising a first vector of the invention comprising a transgene encoding a cytokine (preferably IL10); and a second vector of the invention comprising a transgene encoding a tumour antigen.

In another aspect, the invention provides a product (e.g., a composition or kit) comprising a first vector of the invention comprising a transgene encoding a cytokine (preferably IL10) and a cell comprising a second vector of the invention comprising a transgene encoding a tumour antigen.

In another aspect, the invention provides a product (e.g., a composition or kit) comprising a first cell comprising a first vector of the invention comprising a transgene encoding a cytokine (preferably IL10) and a second cell comprising a second vector of the invention comprising a transgene encoding a tumour antigen.

The composition may be a pharmaceutical composition as disclosed herein.

In another aspect, the invention provides a first vector of the invention comprising a transgene encoding a cytokine (preferably IL10) for use in therapy, wherein the first vector is administered to a subject simultaneously, sequentially or separately in combination with a second vector of the invention comprising a transgene encoding a tumour antigen.

In another aspect, the invention provides a second vector of the invention comprising a transgene encoding a tumour antigen for use in therapy, wherein the second vector is administered to a subject simultaneously, sequentially or separately in combination with a first vector of the invention comprising a transgene encoding a cytokine (preferably IL10).

In another aspect, the invention provides use of a first vector of the invention comprising a transgene encoding a cytokine (preferably IL10) for the manufacture of a medicament, wherein the first vector is administered to a subject simultaneously, sequentially or separately in combination with a second vector of the invention comprising a transgene encoding a tumour antigen.

In another aspect, the invention provides use of a second vector of the invention comprising a transgene encoding a tumour antigen for the manufacture of a medicament, wherein the second vector is administered to a subject simultaneously, sequentially or separately in combination with a first vector of the invention comprising a transgene encoding a cytokine (preferably IL10).

In preferred embodiments, the use in therapy is treatment or prevention of cancer.

In another aspect, the invention provides a method of treating or preventing cancer comprising administering a first vector of the invention comprising a transgene encoding a cytokine (preferably IL10) and a second vector of the invention comprising a transgene encoding a tumour antigen to a subject in need thereof. The first vector and the second vector may be administered, for example, simultaneously, sequentially or separately.

In some embodiments, the first vector and/or the second vector is administered by intravenous injection, intraportal injection or intrahepatic artery injection.

In another aspect, the invention provides a cell comprising a first vector of the invention comprising a transgene encoding a cytokine (preferably IL10) and/or a second vector of the invention comprising a transgene encoding a tumour antigen.

### Exemplary Vectors

In preferred embodiments, the vector comprises from 5′ to 3′: an MRC1 promoter, a transgene, and one or more miRNA target sequence as defined herein. In other preferred embodiments, the vector comprises from 5′ to 3′: a MRC1 enhancer, an MRC1 promoter, a transgene, and one or more miRNA target sequence as defined herein.

In some embodiments, the vector comprises from 5′ to 3′: an MRC1 promoter, a transgene encoding IFNalpha, and one or more miRNA target sequence that suppresses transgene expression in hepatocytes and/or liver sinusoidal endothelial cells and/or splenic phagocytes.

In some embodiments, the vector comprises from 5′ to 3′: a MRC1 enhancer, an MRC1 promoter, a transgene encoding IFNalpha, and one or more miRNA target sequence that suppresses transgene expression in hepatocytes and/or liver sinusoidal endothelial cells and/or splenic phagocytes.

In some embodiments, the vector comprises from 5′ to 3′: a MRC1 enhancer, an MRC1 promoter, a Kozak sequence, a transgene encoding IFNalpha, a WPRE, and one or more miRNA target sequence that suppresses transgene expression in hepatocytes and/or liver sinusoidal endothelial cells and/or splenic phagocytes.

In some embodiments, the vector comprises from 5′ to 3′: a nucleotide sequence which is at least 70% identical to SEQ ID NO: 31 or a fragment thereof; a nucleotide sequence which is at least 70% identical to SEQ ID NO: 34 or a fragment thereof; and a nucleotide sequence which is at least 70% identical to SEQ ID NO: 36 or a fragment thereof.

In some embodiments, the vector comprises from 5′ to 3′: a nucleotide sequence which is at least 70% identical to SEQ ID NO: 32 or a fragment thereof; a nucleotide sequence which is at least 70% identical to SEQ ID NO: 31 or a fragment thereof; a nucleotide sequence which is at least 70% identical to SEQ ID NO: 34 or a fragment thereof; and a nucleotide sequence which is at least 70% identical to SEQ ID NO: 36 or a fragment thereof.

In some embodiments, the vector comprises from 5′ to 3′: a nucleotide sequence which is at least 70% identical to SEQ ID NO: 32 or a fragment thereof; a nucleotide sequence which is at least 70% identical to SEQ ID NO: 31 or a fragment thereof; a nucleotide sequence which is at least 70% identical to SEQ ID NO: 33 or a fragment thereof; a nucleotide sequence which is at least 70% identical to SEQ ID NO: 34 or a fragment thereof; a nucleotide sequence which is at least 70% identical to SEQ ID NO: 35 or a fragment thereof; and a nucleotide sequence which is at least 70% identical to SEQ ID NO: 36 or a fragment thereof.

In some embodiments, the vector comprises from 5′ to 3′: an MRC1 promoter, a transgene encoding IL10, and one or more miRNA target sequence that suppresses transgene expression in hepatocytes and/or liver sinusoidal endothelial cells and/or splenic phagocytes.

In some embodiments, the vector comprises from 5′ to 3′: a MRC1 enhancer, an MRC1 promoter, a transgene encoding IL10, and one or more miRNA target sequence that suppresses transgene expression in hepatocytes and/or liver sinusoidal endothelial cells and/or splenic phagocytes.

In some embodiments, the vector comprises from 5′ to 3′: a MRC1 enhancer, an MRC1 promoter, a Kozak sequence, a transgene encoding IL110, a WPRE, and one or more miRNA target sequence that suppresses transgene expression in hepatocytes and/or liver sinusoidal endothelial cells and/or splenic phagocytes.

In some embodiments, the vector comprises from 5′ to 3′: a nucleotide sequence which is at least 70% identical to SEQ ID NO: 31 or a fragment thereof; a nucleotide sequence which is at least 70% identical to SEQ ID NO: 39 or a fragment thereof; and a nucleotide sequence which is at least 70% identical to SEQ ID NO: 36 or a fragment thereof.

In some embodiments, the vector comprises from 5′ to 3′: a nucleotide sequence which is at least 70% identical to SEQ ID NO: 32 or a fragment thereof; a nucleotide sequence which is at least 70% identical to SEQ ID NO: 31 or a fragment thereof; a nucleotide sequence which is at least 70% identical to SEQ ID NO: 39 or a fragment thereof; and a nucleotide sequence which is at least 70% identical to SEQ ID NO: 36 or a fragment thereof.

In some embodiments, the vector comprises from 5′ to 3′: a nucleotide sequence which is at least 70% identical to SEQ ID NO: 32 or a fragment thereof; a nucleotide sequence which is at least 70% identical to SEQ ID NO: 31 or a fragment thereof; a nucleotide sequence which is at least 70% identical to SEQ ID NO: 33 or a fragment thereof; a nucleotide sequence which is at least 70% identical to SEQ ID NO: 39 or a fragment thereof; a nucleotide sequence which is at least 70% identical to SEQ ID NO: 35 or a fragment thereof; and a nucleotide sequence which is at least 70% identical to SEQ ID NO: 36 or a fragment thereof.

In some embodiments, the vector comprises from 5′ to 3′: an MRC1 promoter, a transgene encoding IL12, and one or more miRNA target sequence that suppresses transgene expression in hepatocytes and/or liver sinusoidal endothelial cells and/or splenic phagocytes.

In some embodiments, the vector comprises from 5′ to 3′: a MRC1 enhancer, an MRC1 promoter, a transgene encoding IL12, and one or more miRNA target sequence that suppresses transgene expression in hepatocytes and/or liver sinusoidal endothelial cells and/or splenic phagocytes.

In some embodiments, the vector comprises from 5′ to 3′: a MRC1 enhancer, an MRC1 promoter, a Kozak sequence, a transgene encoding IL112, a WPRE, and one or more miRNA target sequence that suppresses transgene expression in hepatocytes and/or liver sinusoidal endothelial cells and/or splenic phagocytes.

In some embodiments, the vector comprises from 5′ to 3′: a nucleotide sequence which is at least 70% identical to SEQ ID NO: 31 or a fragment thereof; a nucleotide sequence which is at least 70% identical to SEQ ID NO: 40 or a fragment thereof; and a nucleotide sequence which is at least 70% identical to SEQ ID NO: 36 or a fragment thereof.

In some embodiments, the vector comprises from 5′ to 3′: a nucleotide sequence which is at least 70% identical to SEQ ID NO: 32 or a fragment thereof; a nucleotide sequence which is at least 70% identical to SEQ ID NO: 31 or a fragment thereof; a nucleotide sequence which is at least 70% identical to SEQ ID NO: 40 or a fragment thereof; and a nucleotide sequence which is at least 70% identical to SEQ ID NO: 36 or a fragment thereof.

In some embodiments, the vector comprises from 5′ to 3′: a nucleotide sequence which is at least 70% identical to SEQ ID NO: 32 or a fragment thereof; a nucleotide sequence which is at least 70% identical to SEQ ID NO: 31 or a fragment thereof; a nucleotide sequence which is at least 70% identical to SEQ ID NO: 33 or a fragment thereof; a nucleotide sequence which is at least 70% identical to SEQ ID NO: 40 or a fragment thereof; a nucleotide sequence which is at least 70% identical to SEQ ID NO: 35 or a fragment thereof; and a nucleotide sequence which is at least 70% identical to SEQ ID NO: 36 or a fragment thereof.

### Variants, Derivatives, Analogues, and Fragments

In addition to the specific proteins and nucleotides mentioned herein, the invention also encompasses variants, derivatives, and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

Typically, a variant may have a certain identity with the wild type amino acid sequence or the wild type nucleotide sequence.

In the present context, a variant sequence is taken to include an amino acid sequence which may be at least 50%, 55%, 65%, 75%, 85% or 90% identical, suitably at least 95%, 96% or 97% or 98% or 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, 55%, 65%, 75%, 85% or 90% identical, suitably at least 95%, 96% or 97% or 98% or 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs detailed herein refers to a sequence which has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410), EMBOSS Needle (Madeira, F., et al., 2019. Nucleic acids research, 47(W1), pp. W636-W641) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives, and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Cells

In one aspect, the present invention provides a cell comprising the vector of the invention. The cell may be an isolated cell. The cell may be a human cell, suitably an isolated human cell. The cell may be any cell type known in the art.

The cell may comprise the first and/or second vector of the invention.

### Method of Making a Cell

The vector of the present invention may be introduced into cells using a variety of techniques known in the art, such as transfection, transduction and transformation. Suitably, the vector of the present invention is introduced into the cell by transfection or transduction.

In one aspect, the present invention provides a method of making the cell of the invention. The method may comprise introducing the vector of the invention into the cell, for example by transfection or transduction.

Suitably, the cell may be from a sample (e.g. peripheral blood, bone marrow or umbilical cord blood) isolated from a subject. The cell may be further separated from the sample by any suitable method.

The cell of the present invention may be generated by a method comprising the following steps:
(i) isolation of a cell-containing sample from a subject or provision of a cell-containing sample; and
(ii) transduction or transfection of the cell-containing sample with the vector of the invention, to provide a population of engineered cells.

The cells may be cultured prior to, or after, introducing the vector of the invention. The steps may be performed in a closed and sterile cell culture system.

### Hematopoietic Stem/Progenitor Cells and Differentiated Cells

Suitably, the cell may be a hematopoietic stem cell (HSC), a hematopoietic progenitor cell (HPC) (e.g. a myeloid/monocyte-committed progenitor cell) or a differentiated cell (e.g. a macrophage or a monocyte). Suitably, the cell may be autologous and/or allogenic to a subject.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells (HPCs) have the capacity to differentiate into a specific type of cell. In contrast to stem cells however, they are already far more specific: they are pushed to differentiate into their “target” cell. A difference between HSCs and HPCs is that HSCs can replicate indefinitely, whereas HPCs can only divide a limited number of times.

Differentiated cells have become more specialised in comparison to a stem cell or progenitor cell. Differentiated cells includes differentiated cells of the haematopoietic lineage such as monocytes, macrophages, neutrophils, basophils, eosinophils, erythrocytes, megakaryocytes/platelets, dendritic cells, T-cells, B-cells and NK-cells. For example, differentiated cells of the haematopoietic lineage can be distinguished from HSCs and HPCs by detection of cell surface molecules which are not expressed or are expressed to a lesser degree on undifferentiated cells (HSCs and HPCs). Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T), CD56 (NK).

The cell of the present invention may be used for adoptive cell transfer. As used herein the term “adoptive cell transfer” refers to the administration of a cell population to a patient. The cell may be isolated from a subject, the vector of the invention may be introduced by a method described herein before the cell is administered to the patient.

Adoptive cell transfer may be allogenic or autologous. By “autologous cell transfer” it is to be understood that the starting population of cells is obtained from the same subject as that to which the transduced cell population is administered. Autologous transfer is advantageous as it avoids problems associated with immunological incompatibility and is available to subjects irrespective of the availability of a genetically matched donor. By “allogeneic cell transfer” it is to be understood that the starting population of cells is obtained from a different subject as that to which the transduced cell population is administered. Optionally, the donor will be genetically matched to the subject to which the cells are administered to minimise the risk of immunological incompatibility. Alternatively, the donor may be mismatched and unrelated to the patient. Suitable doses of transduced cell populations are such as to be therapeutically and/or prophylactically effective. The dose to be administered may depend on the subject and condition to be treated, and may be readily determined by a skilled person.

### Producer Cells and Packaging Cells

Suitably, the cell may be a producer cell. The term “producer cell” includes a cell that produces viral particles, after transient transfection, stable transfection or vector transduction of all the elements necessary to produce the viral particles or any cell engineered to stably comprise the elements necessary to produce the viral particles. Suitable producer cells will be well known to those of skill in the art. Suitable producer cell lines include HEK 293 (e.g. HEK 293T), HeLa, and A549 cell lines.

Suitably, the cell may be a packaging cell. The term “packaging cell” includes a cell which contains some or all of the elements necessary for packaging an infectious recombinant virus. The packaging cell may lack a recombinant viral vector genome. Typically, such packaging cells contain one or more vectors which are capable of expressing viral structural proteins. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell”. Suitable packaging cells will be well known to those of skill in the art.

In some embodiments, the cell is genetically engineered to decrease expression of CD47 and/or HLA on the surface of the cell. In some embodiments, the cell comprises a genetically engineered disruption of a gene encoding CD47, and/or a gene encoding 32-microglobulin, and/or one or more genes encoding an MHC-I a chain. The cell may comprise genetically engineered disruptions in all copies of the gene encoding CD47. The expression of CD47 and/or HLA on the surface of the cell may be decreased such that the cell is substantially devoid of surface-exposed CD47 and/or HLA molecules. In some embodiments, the cell does not comprise any surface-exposed CD47 and/or HLA molecules.

In one aspect, the present invention provides a method of making the viral vector particle of the invention. The method may comprise culturing a viral particle producer or packaging cell comprising the vector of the invention under conditions suitable for the production of the viral particles. The method may comprise: (a) introducing the vector of the invention into a viral particle producer or packaging cell, for example by transfection or transduction; and (b) culturing the cell under conditions suitable for the production of the viral particles. Such conditions will be well known to those of skill in the art.

### Pharmaceutical Compositions

In one aspect, the present invention provides pharmaceutical composition comprising the vector of the invention or the cell of the invention.

A pharmaceutical composition is a composition that comprises or consists of a therapeutically effective amount of a pharmaceutically active agent i.e. the vector. It preferably includes a pharmaceutically acceptable carrier, diluent or excipient (including combinations thereof).

In some embodiments the pharmaceutical composition is a cancer vaccine. A “cancer vaccine” is a vaccine that either treats existing cancer or prevents development of cancer.

By “pharmaceutically acceptable” is included that the formulation is sterile and pyrogen free. The carrier, diluent, and/or excipient must be “acceptable” in the sense of being compatible with the vector and not deleterious to the recipients thereof. Typically, the carriers, diluents, and excipients will be saline or infusion media which will be sterile and pyrogen free, however, other acceptable carriers, diluents, and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The vector, cell, or pharmaceutical composition according to the present invention may be administered in a manner appropriate for treating and/or preventing the diseases described herein. The quantity and frequency of administration will be determined by such factors as the condition of the subject, and the type and severity of the subject's disease, although appropriate dosages may be determined by clinical trials. The pharmaceutical composition may be formulated accordingly.

The vector, cell or pharmaceutical composition according to the present invention may be administered parenterally, for example, intravenously, or by infusion techniques. The vector, cell or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The vector, cell or pharmaceutical composition according to the present invention may be administered systemically, for example by intravenous injection.

The vector, cell or pharmaceutical composition according to the present invention may be administered locally, for example by targeting administration to the liver. Suitably, the vector, cell or pharmaceutical composition may be administered by intraportal injection or by intrahepatic artery injection.

The pharmaceutical compositions may comprise vectors or cells of the invention in infusion media, for example sterile isotonic solution. The pharmaceutical composition may be enclosed in ampoules, disposable syringes or multiple dose vials made of glass or plastic.

The vector, cell or pharmaceutical composition may be administered in a single or in multiple doses. Particularly, the vector, cell or pharmaceutical composition may be administered in a single, one off dose. The pharmaceutical composition may be formulated accordingly.

The vector, cell or pharmaceutical composition may be administered at varying doses (e.g. measured in vector genomes (vg) per kg). The physician in any event will determine the actual dosage which will be most suitable for any individual subject and it will vary with the age, weight and response of the particular subject.

The pharmaceutical composition may further comprise one or more other therapeutic agents. The vector, cell or pharmaceutical composition may be administered in combination with one or more other therapeutic agents.

The invention further includes the use of kits comprising the vector, cells and/or pharmaceutical composition of the present invention. Preferably said kits are for use in the methods and used as described herein, e.g., the therapeutic methods as described herein. Preferably said kits comprise instructions for use of the kit components.

### Methods for Treating and/or Preventing Disease

In one aspect, the present invention provides the vector, cell or pharmaceutical composition according to the present invention for use as a medicament.

In a related aspect, the present invention provides use of the vector, cell or pharmaceutical composition according to the present invention in the manufacture of a medicament.

In a related aspect, the present invention provides a method of administering the vector, cell or pharmaceutical composition according to the present invention to a subject in need thereof. Suitably, the subject is a human subject.

### Cancer

The vector, cell or pharmaceutical composition according to the present invention may be used to prevent or treat cancer in a subject. Suitably, the subject is a human subject.

In one aspect, the present invention provides the vector, cell or pharmaceutical composition according to the present invention for use in preventing or treating cancer.

In a related aspect, the present invention provides use of the vector, cell or pharmaceutical composition according to the present invention for the manufacture of a medicament for preventing or treating cancer.

In a related aspect, the present invention provides a method of preventing or treating cancer comprising administering the vector, cell or pharmaceutical composition according to the present invention to a subject in need thereof.

### Liver Metastases

In some embodiments the cancer is liver cancer, for example secondary liver cancer (e.g. liver metastases).

In some embodiments the subject has or is at risk of developing a secondary liver cancer (e.g. liver metastases) and the vector, cell or pharmaceutical composition is used to prevent or treat the secondary liver cancer.

In some embodiments the subject has a primary cancer (e.g. of colorectal, pancreatic or breast origin) and the vector, cell or pharmaceutical composition is used to prevent or treat a secondary liver cancer (e.g. liver metastases).

Metastasis is the development of secondary malignant growths at a distance from a primary site of cancer. Metastases most commonly develop when cancer cells break away from the main tumour and enter the bloodstream or lymphatic system.

The liver is one of the most common sites for cancer metastasis, accounting for nearly 25% of all cases. The high frequency of liver involvement in metastatic disease can be explained by the different hypotheses of metastatic spread. The double blood supply of the liver by the portal vein and the hepatic artery facilitates entrapment of circulating cancer cells, according to the “mechanical or hemodynamic hypothesis”, which explains the high incidence of liver metastases in patients with gastrointestinal carcinomas. On the other hand, some primary tumours selectively target the liver as a metastatic location, according to the “seed-and-soil” hypothesis, examples are patients with uveal melanoma with a loss of chromosome 3, and patients with breast cancer with the human growth factor receptor 2 (HER-2) positivity in combination with estrogen (ER) and progesterone receptor (PR) positivity (de Ridder, J., et al., 2016. Oncotarget, 7(34), p. 55368).

The majority of liver metastases are carcinomas, particularly adenocarcinoma. The primary tumour may be any primary tumour, and the primary tumour may be unknown. However, most common primary tumours in patients with adenocarcinoma are from colorectal, pancreatic or breast origin (de Ridder, J., et al., 2016. Oncotarget, 7(34), p. 55368).

Subjects may be diagnosed with liver metastases by any suitable method known to those of skill in the art. For example, subjects may be diagnosed by CT imaging with a hepatic protocol, colonoscopy, and EGD.

The vector, cell or pharmaceutical composition of the present invention may be used for treating or preventing liver metastases in combination with any other suitable therapy. For example, in combination with surgical resection of hepatic metastases and/or chemotherapy.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1—Platform to Express Therapeutic Gene Products from MRC1-Expressing Phagocytic Cells in the Liver and Spleen

### Results

We designed and produced a lentiviral vector (LV) driving transgene expression from a M2-like macrophage specific promoter, i.e., the MRC1 promoter. In order to identify the main regulatory feature of the mouse Mrc1, we selected a 1.8 kb sequence immediately upstream to the Mrc1 open reading frame (ORF) and incorporated it in an LV upstream to a GFP coding sequence originating the Mrc1.GFP LV. We then transduced bone marrow-derived macrophages (BMDMs) with either the Mrc1.GFP LV or an LV driving GFP expression under the control of the constitutively expressed human PGK promoter (PGK.GFP LV). Upon stimulation of transduced BMDMs with either LPS (M1 polarization) or IL4 (M2 polarization) we found that the Mrc1.GFPdrove transgene expression in M2, but not in M1 BMDMs (FIG. 1a). Whereas the PGK.GFP LV drove transgene expression independently of BMDM polarization status. We then delivered the Mrc1.GFP LV systemically to immunodeficient mice and analysed GFP expression in distinct biological compartments by using flow cytometry. We observed selective transgene expression in KCs, and to a lesser extent in MRC1+ splenic macrophages and LSECs (FIG. 1c). Of note, GFP expression in the blood, lung and bone-marrow was not detected. In agreement with these data, we observed integrated LV copies in liver cells and in the spleen, but not in other biological compartments (FIG. 1d).

We investigated if adding enhancer sequences normally found in the Mrc1 locus to the LV design could by employed to enhance the transcriptional function of the Mrc1 promoter upon macrophage polarization. To this aim, mouse enhancer 6 (SEQ ID NO: 27) was inserted upstream to a LV expressing GFP from the Mrc1 promoter. Immortalized Kupffer cells (iKCs) were then transduced with the resulting LV and polarized with 50 ng of IL4 for 7 days. We found that the Mrc1 enhancer increased the expression of GFP driven by the Mrc1 promoter when iKCs were polarized with IL4 compared to Mrc1 promoter with no enhancer (FIG. 1b).

To increase the specificity of the Mrc1-driven LV in macrophages, we incorporated miRNA target sequences (miRTs) in the LV design. We assess the ability of the miRTs to abate transgene expression in the target cells by employing a bidirectional LV, which drives the expression of two independent transcripts, i.e. a truncated low affinity nerve growth factor receptor (dLNGFR, an inert membrane protein used as normalizer), and the GFP. Downstream to the GFP sequence in the bidirectional LV, we incorporated microRNA target sequences for miR-126-3p (miRT126 LV), expressed by LSECs, and miR-122-5p (miRT122 LV), expressed by hepatocytes. The miRT LVs were delivered systemically to immunodeficient mice and GFP expression was evaluated in the cells of interest. We observed that miRT126 abated GFP expression in LSECs, while preserving it in KCs (FIG. 2a). In a similar way, miRT122 suppressed the expression of the GFP in hepatocytes, but not in macrophages (FIG. 2b-c). These results indicate that miRT122 and miRT126 in the LV design further fine tune specific transgene expression in macrophages. Building on this result we incorporated miRT122 and miRT126 sequences to the Mrc1.GFP LV design originating the Mrc1.GFP.miRT LV.

We then investigated transgene product expression driven by the Mrc1.GFP.miRT LV in the presence of experimental LMS. To this aim, we delivered systemically either the Mrc1.GFP LV or the Mrc1.GFP.miRT to mice. One-week after LV delivery, we inoculated mCherry-positive MC38 colorectal cancer cells by intrahepatic injection to originate experimental LMS. We found that within the liver, KCs expressed the highest levels of GFP (FIG. 3a). Moreover, in agreement with our previous results, miRTs in the LV design abated transgene expression in LSECs. We found that transgene product expression, measured as GFP, was higher in areas located in proximity to liver metastases than in other areas (FIG. 3b). We then measured LV integration in inguinal lymph nodes, small intestine, lungs, brain, liver and spleen. All these biological compartments host resident macrophage populations that could potentially express GFP upon systemic delivery of the Mrc1.GFP LVs. In agreement with our previous result, we found that only the spleen and the liver showed detectable integrated LV copies (FIG. 3c). In a similar way, GFP expression above the background level was detected only in phagocytic cells, such as the splenic macrophages and KCs, but not in other cell populations (FIG. 3d, e). These results indicate that the Mrc1/miRT-driven LV can be used to selectively promote transgene product expression in phagocytic cells, especially in the presence of LMS, and to a lesser extent in splenic MRC1-positive macrophages.

In order to validate the Mrc1/miRT-driven LV platform as a tool to express proteins of interest in the liver, we delivered molecules with anti-tumor activity to liver metastases. As molecule with anti-tumor activity we employed Interferon-α (IFNα), a cytokine that can drive anti-tumor immune and angiostatic effects. We generated an LV hosting a mouse type I, IFNα, cDNA under the control of the Mrc1 promoter and miRT122/miRT126, originating the Mrc1.IFNα.miRT LV. As control for these experiments, we generated an LV containing the regulatory features of the Mrc1.IFNα.miRT LV (i.e., Mrc1 promoter and miRT122/126), but lacking IFNα cDNA, originating the ORFless LV. We then assess the capacity of the platform to express IFNα in vivo. To this aim we delivered to immunocompetent C57BL6 mice either the Mrc1.IFNα.miRT LV or the ORFless LV, and monitor IFNα in the plasma over a period of time. We observed that mice treated with the Mrc1.IFNα.miRT LV expressed robust and sustained levels of IFNα with no sign of hepatotoxicity, neutropenia, nor strong leukopenia (FIG. 4a-c).

We designed a LV to drive the expression of IL10 and IL12. To this aim mouse IL10 cDNA (SEQ ID NO: 39) was introduced at the place of the IFNα in the Mrc1.IFNα.miRT LV originating the Mrc1.IL10.miRT LV. The resulting LV was used to transduce the P388D1 monocytic cell line at distinct multiplicity of infection (MOI). IL10 concentration in the transduced P388D1-conditioned cell culture medium was measured by using ELISA. We found detectable levels in cell culture medium of Mrc1.IL10.miRT LV-transduced P388D1 (FIG. 4d). To assess the capacity of the KC LV to express IL10 in vivo, a single dose ranging from 1*10⁷ to 5*10⁷ TU per mouse of Mrc1.IL10.miRT LV was delivered to 5 week old mice i.v. Plasma was collected after 21 days from treatment. We found higher concentration of IL10 in the plasma of Mrc1.IL10.miRT LV-treated mice than in untreated control mice (FIG. 4e). Indicating that KC LV can be used to drive IL10 expression in vivo. We then generated a DNA encoding for a 557 amino acid a single chain functional IL12 molecule (SEQ ID NO: 40), where: 1) amino acids from 1 to 23 are the signal peptide of mouse IL12 beta subunit, 2) amino acids from 24 to 28 are a linker sequence composed of the amino acidic sequence AGQLM, 3) amino acids from 29 to 340 are amino acids 23 to 335 of beta subunit of mouse IL12, 4) amino acids from 341 to 360 are a linker sequence composed of the amino acidic sequence RRAGGGGSGGGGSGGGGSRT, 5) (SEQ ID NO: 42) amino acids from 361 to 553 are mouse IL12 subunit alpha (isoform 1) from amino acids 44 to 236, 6) amino acids from 554 to 557 are a termination sequence composed of the amino acidic sequence TRAS. Single chain IL12 cDNA was then inserted at the place of the IFNα in the Mrc1.IFNα.miRT LV originating the Mrc1.IL12.miRT LV. We delivered a single dose of 2*10⁶ TU per mouse of Mrc1.IL12.miRT LV to 6 week old mice i.v. and collected plasma after 10 days from treatment. We found higher levels of IL112 in the plasma from Mrc1.IL12.miRT LV-treated mice than in Mrc1.ORFless.miRT (ORFless)-treated ones (FIG. 4f). We also analysed whole blood cell (WBC) count at different time points and found that IL12 was well tolerated by treated mice since WBC remain stable during the period of observation of 40 days (FIG. 4g). Altogether these data indicate that the KC LV platform can drive the expression of virtually any therapeutic cytokine or protein in vivo, and that this treatment can be well tolerated.

In order to investigate if Mrc1.IFNα.miRT could be used as therapeutic intervention for tumors present in the liver, we treated mice hosting experimental LMS with the Mrc1.IFNα.miRT or the ORFless LVs (FIG. 5a). To this aim, we first inoculated MC38 cells by intrahepatic injection in syngeneic C57BL6 mice. Once LMS were established, we delivered systemically to the mice either the Mrc1.IFNα.miRT or the ORFless LV. We found that mice treated with the Mrc1.IFNα.miRT LV displayed sustained levels of IFNα in the plasma (FIG. 5b). We monitored tumor growth by magnetic resonance imaging (MRI). We found that IFNα expression delayed tumor progression and one out of 10 mice completely responded to the treatment (FIG. 5c). We obtained similar results employing a 10-fold lower LV dose (FIG. 5d,e). In order to investigate whether the mice that rejected the LMS developed protected immunity we rechallenge the mice with subcutaneous MC38 cells. The mouse that previously rejected the MC38 liver metastasis remained tumor free for longer period of time than the (PBS-treated) mice (FIG. 5f), suggesting that the immune system may eliminate most of the MC38 cells, delaying tumor progression in the complete responder mouse.

To further investigate the role played by the immune system in delaying MC38 tumor growth, we employed MC38 cells expressing a surrogate tumor antigen, i.e. chicken ovalbumin (OVA). In agreement with our previous result, the Mrc1.IFNα.miRT LV promoted sustained and robust IFNα expression that could be detected in the plasma of the treated mice (FIG. 6a). We observed delayed tumor growth in mice treated with the Mrc1.IFNα.miRT LV and three out of 10 mice rejected the tumor (FIG. 6b). Importantly, the percentage of OVA reactive and therefore cancer cell specific CD8 T cells was significantly increased in the Mrc1.IFNα.miRT LV group indicating activation of adaptive immunity (FIG. 6c). In the Mrc1.IFNα.miRT LV group, TAMs were skewed towards M1-like polarization, whereas the proportion of M2-like TAMs was reduced (FIG. 6d). Altogether, systemic delivery of Mrc1.IFNα.miRT LV to mice hosting MC38-based experimental LMS delayed tumor growth, reprogrammed TAMs and promoted adaptive immunity.

To assess if IL2 expression by the KC LV can be exploited therapeutically, we delivered i.v. a single dose of 2*10⁶ TU per mouse of Mrc1.IL12.miRT LV or Mrc1.ORFless.miRT LV as control to 6 week old mice. We then inoculated subcutaneous tumors based on MC38 cells constitutively expressing ovalbumin (OVA, MC38.OVA) at day 14 from the LV treatment. After 27 days from tumor inoculation mice were euthanized. We found elevated levels of cancer cell specific (pentamer+) CD8 T cells in the liver of Mrc1.IL12.miRT LV-treated mice compared to Mrc1.ORFless.miRT LV-treated controls. We also found elevated levels of CD44+ pentamer+CD8 T cells in the spleen of IL2-treated mice indicating activation of adaptive immune system (FIG. 6e). We found that IL12 expression delayed tumor growth and 3 out of 5 mice resulted tumor free at the end-point of the experiment (FIG. 6e). These data indicate that the KC LV platform can be used to express a single chain IL12 with therapeutic activity in tumors that are located in sites distant from the liver.

As experimental LMS tumour model, intrahepatic deliver of MC38 has some limitations. For example, MC38 cells directly develop in the liver, thus omitting extravasation and spontaneous seeding of the cancer cells, furthermore, malignant transformation occurred through driver mutations that do not fully resemble those observed in human cancer patients. To overcome these limitations, we employed a CRC organoid-based LMS tumor model that better recapitulates the disease as observed in patients. CRC organoids derive from spontaneous genetically engineered mouse CRC, which are endowed with a well-defined set of driver mutations (APC^{Δ716}; Kras^{G12D}; Tgfbr2^{-/-}; Trp53^{R27OH}; Fbxw7^{-/-}). CRC organoids were delivered to mice by intrasplenic injection allowing spontaneous extravasation and seeding of the cancer cells in the in the liver. One-week after LMS formation, mice were treated with either the Mrc1.IFNα.miRT LV or the control ORFless LV. In agreement with our previous results, we observed stable and robust IFNα expression in the plasma of the treated mice (FIG. 7a). We found that Mrc1.IFNα.miRT LV delayed tumor growth compared to ORFless LV and 3 out of 10 mice rejected the LMS (FIG. 7b). We observed that Mrc1.IFNα.miRT LV increased the expression of IFNα-induced genes in the liver and to a higher extent in the metastatic masses of the treated mice, indicating IFNα signalling activation (FIG. 7c). Furthermore, we found that IFNα expression by phagocytic cells, such as KCs, promoted infiltration of CD8 T cells and polarization of macrophages to an M1-like phenotype (FIG. 7d,e). We obtained similar results in an experimental duplicate (FIG. 7f,g).

Building on the fact that the Mrc1-miRT regulated LV drives transgene expression selectively in antigen presenting cell (APCs), we decided to investigate the feasibility of employing this platform as tumor vaccines to promote adaptive immunity against tumor antigens (TAs). To do this, we employed the chicken ovalbumin (OVA) as a surrogate tumor antigen. We first substituted the IFNα coding sequence in the Mrc1.IFNα.miRT with a truncated (to limit extracellular secretion) OVA expressing sequence, originating the Mrc1.OVA.miRT. In order to generate experimental liver metastases, we inoculated MC38 cells, previously transduced with an OVA expressing LV at VCN 3, intra-hepatically in syngeneic C57BL6 mice. Two days after tumor challenge, we delivered systemically, through tail vein injection, either the Mrc1.OVA.miRT LV or the ORFless LV, while a group of mice, which were not challenged with liver metastases, received PBS. We found that the presence of OVA-expressing liver metastases significantly increased the percentage of OVA-specific (pentamer positive) CD8 T cells compared to mice with no tumors (FIG. 8a). Of note, Mrc1.OVA.miRT increased the number of circulating OVA-specific PD1 expressing CD8 T cells, indicating that tumor vaccination using the Mrc1-miRT regulated LVs can enhance activation and exhaustion of CD8 T cells (FIG. 8b). We observed that liver of Mrc1.OVA.miRT treated mice displayed a very high number of OVA-specific CD8 T cells (FIG. 8c), which may protect the liver for future metastatic seeding. In summary, Mrc1.OVA.miRT LV strongly increases the number of cancer cell specific T cells indicating that the platform can be used to promote adaptive immunity against specific TAs.

### Materials and Methods

### Lentiviral Vector Construction

In order to obtain the Mrc1 promoter sequence (Seq 1), mouse BMDMs were obtained as described below, and DNA was extracted using the GEL extraction kit (Qiagen) as indicated by the manufacturer. A sequence corresponding to a putative Mrc1 promoter was amplified by PCR using the Pfu ultra II (Agilent Technologies) polymerase as indicated by the manufacture. Primers are described below. PCR was run in SensoQuest GmbH labcycler and purified using High Pure PCR product purification kit (Qiagen). After running the amplicons in 1% agarose gel, they were extracted using the Jetquick gel extraction spin kit (Genomed). The amplicon was then cloned using Xhol and Agel restriction sites replacing a PGK sequence upstream to a GFP sequence in a PGK.GFP LV described previously (Squadrito, M. L., et al., Nat Methods, 2018. 15(3): p. 183-186).

FWD:
(SEQ ID NO: 48)
AACTCGAGCCGAGCTCTGAAATGGATGCTT
REV:
(SEQ ID NO: 49)
AACCGGTTCCAATGAGGGAGAAGAGGGAG
Seq 1. Mrc1 promoter
(SEQ ID NO: 2)
CTCGAGCCGAGCTCTGAAATGGATGCTTCAAGGATTTGAAGAGAC
ACCAGAAGTGAAAAACGTGCTATTTTCCCACAGTTCCTGGCAATA
CAAAGATTGTTTTAAGGCCTATGGAAATTCCTCTTCCTCCGTTAC
CTGAAATTACAGATTTGTGTTGACTTGCTCACCCCTCCTAACCTG
ATAAAATCTTCCAATAAGATAAAAATGATGGAGACAAATCCTTTG
TGGGATGTTGGACTTCACTTTATATCACATCCAGCGTCTCGTTAC
TGATTCTGATTTTATTCCTGTGCATGTAAGACACGTTGACATAAT
AAAACCATGGATATACAGATGCCTGCAATTCAGTTAACTCTTTTT
TTTCCTCTTCAAATAAGTCAAAGCAAACCCCAATTAGGCAAAACA
ATTTGAATGGCTTGCATTTAAAAGACCAATTAAAACATTTTTTGG
TCAGCAAGCATGATGGGACACACTTATAATCCCAGCTCTCAGAAA
GTCAAAACAGAGGAACCAAGAATTCAAGGCCAGCCTGCGCTACAA
ACGCAAGACTGTTTCGGTGTTCCTGTGATAAGTCAGTTACGCAGT
GATTGAAAAGGAAACGTTTGCAGCCTCTCACCAGTTGTGGGAGAA
TTTTCTTTGTCAGTTAAGCCTTGATAGAATGAAAAAGAACGGTGG
GTCCCTTCTCAGAATCTTCCTAATTTAGGCTTTTTAAAAAGAAAA
TTCTTGAGAGAAACCACAGCTTATTGGGAAATGAGTGTGTACCTG
CCTCAGCGTGGATGGGTCTGAACAGCTTTTCACTTGAAGGTAAAC
CATCTGTTTACAACTTCTAAGTCGCCAGTGTTTCCAGAGCTTCTT
TTTGAAACGATGACATTTCCCACGCTCCAGTTTCAGGTCTTCCCT
GACTAACCACAAATATCCATTTCTAAATATTCTTAATTCTTGTTG
AACGTCTGGAAAAAAAAAATCAGTGTTTAGGTGGGTTGTGTGGTG
CTTTGTGAACGACCCTGCAAAATCATGAAGACGAAACCCCACTGT
CATCGAATCAACAAGCAACTTTTGGACTCAAGCCAGGCTTTCTTT
TGCAAGAGAGAGAGAGAGGTCTTCCCTTTTTCAAACTCTGAGGAC
TGTAATGGTTGAGGCCTGGCAGCGAACCGACAACAAAGCTATTGC
CACTATTTCCTCTGGCTTTCTAAGGAAAGCTGCTAGAACTTTCTA
TCCCTGGGCTTCATTGAGGTTGTCTTAAAATTAACTTCTGTCATT
TTCCTTCTAGAGACAGGGGCAAAACTCTACGTGAACCATACCTTT
GATCCTTTCCAAGGAGTGTGTGTGTGTGTGTGTGTGTGTGTGTGT
GTGTGTGTGTGTGTGTGTGTTGGTGCTCGGGCTCTAAGCCTGAGC
AGGAAGAGCTTCTGATGCTTTCCAGCGAGTGTCCTCCCTTTCTGA
CTGTAGAATTGTGGGTGAGAGCCTCCACAGCTGCCTCCTGGAGAC
TTTTTCCCACCCAGATAATGGCCTCCGTTTGGTTACTGCCCAGCA
CCTGTGGAGAGCTCAGCAGGGCTGCCTCTCCCTGCTGCTCATGGC
CTGGGTCCTCACTTCTCCCCACTTCCTGCGTTTTCTCCTCTCCTA
CACATGTTCCTCTCTCCCCTTCCTCCTGTGCCTTAGCTTACGAAG
CAAAGTTGTAACTTTGAATTCCTGTTTTTCTAACCGCCCCCATGT
GACAGGATATCTCTCAATTGGAGGGTTTTCCTAAATTCAGGAGTC
CTTTAAAAGGGACAGCTTCCTCTGTCCTCCTTTTCAGTCAGGCAG
CTCCCAGACCTTGGACTGAGCAAAGGGGCAACCTGGGGACCTGGT
TGTATTCTTTGCCTTTCCCAGTCTCCCTCTTCTCCCTCATTGGAA
CCGGT

In order to obtain miRT sequences, the miRT sequences for miRT-122 (Seq 2) and miRT-126 (Seq 3) were inserted downstream to the GFP in a bidirectional LV described previously (Annoni, A., et al., Blood, 2009. 114(25): p. 5152-61).

Seq 2. miRT-122 4 x miRT
(SEQ ID NO: 5)
TCTAGATAAACAAACACCATTGTCACACTCCATTCGAAACAAACA
CCATTGTCACACTCCAACGCGTACAAACACCATTGTCACACTCCA
ATGCATACAAACACCATTGTCACACTCCACCCGGGTCGAGCTCGG
TACC
Seq 3. miRT-126 4 x miRT
(SEQ ID NO: 6)
GGTACCAGCAAACGCATTATTACTCACGGTACGACCATCGCATTA
TTACTCACGGTACGAACTTCGCATTATTACTCACGGTACGACGAA
CGCATTATTACTCACGGTACGACACGTGTCGGTACC

The sequence containing miRT-122 and miRT-126 in tandem (Seq 4) was then cloned in the Mrc1.GFP LV using Kpnl originating the Mrc1.GFP.miRT LV.

Seq 4. miRT-122 / miR126 4 x miRT
(SEQ ID NO: 7)
GGTACCAGCGCTACAAACACCATTGTCACACTCCAACATACAAACA
CCATTGTCACACTCCAGATTACAAACACCATTGTCACACTCCACA
GAACAAACACCATTGTCACACTCCAGTTTAAACGCATTATTACTC
ACGGTACGACCATCGCATTATTACTCACGGTACGAACTTCGCATT
ATTACTCACGGTACGACGAACGCATTATTACTCACGGTACGACAC
GTGTCGGTACC

A DNA sequence encoding for IFNα (Seq 5) was obtained from a previously described construct (Escobar, G., et al., Nat Commun, 2018. 9(1): p. 2896) and cloned at the place of the GFP in the Mrc1.GFP.miRT LV using Agel and Sall originating the Mrc1.IFNα.miRT LV respectively.

Seq 5. IFNα
(SEQ ID NO: 50)
ACCGGTCAGTCCTCCGACAGACTGAGTCGCCCGGGGGGGATCCAC
CGGCATGGCTAGGCTCTGTGCTTTCCTGATGGTCCTGGCGGTGCT
GAGCTACTGGCCAACCTGCTCTCTAGGATGTGACCTTCCTCAGAC
TCATAACCTCAGGAACAAGAGAGCCTTGACACTCCTGGTACAAAT
GAGGAGACTCTCCCCTCTCTCCTGCCTGAAGGACAGGAAGGACTT
TGGATTCCCGCAGGAGAAGGTGGATGCCCAGCAGATCAAGAAGGC
TCAAGCCATCCCTGTCCTGAGTGAGCTGACCCAGCAGATCCTGAA
CATCTTCACATCAAAGGACTCATCTGCTGCATGGAATACAACCCT
CCTAGACTCATTCTGCAATGACCTCCACCAGCAGCTCAATGACCT
GCAAGGCTGTCTGATGCAGCAGGTGGGGGTGCAGGAATTTCCCCT
GACCCAGGAAGATGCCCTGCTGGCTGTGAGGAAATACTTCCACAG
GATCACTGTGTACCTGAGAGAGAAGAAACACAGCCCCTGTGCCTG
GGAGGTGGTCAGAGCAGAAGTCTGGAGAGCCCTGTCTTCCTCTGC
CAATGTGCTGGGAAGACTGAGAGAAGAGAAATGAGTCGAC

A DNA sequence coding chicken ovalbuimin (Seq 6) was cloned downstream to the PGK promoter of a LV previously described (Squadrito, M. L., et al., Nat Methods, 2018. 15(3): p. 183-186) using BamHI and Sall restriction sites.

Seq 6. OVA
(SEQ ID NO: 51)
GGATCCGCCACCATGGGCTCCATCGGCGCAGCAAGCATGGAATTT
TGTTTTGATGTATTCAAGGAGCTCAAAGTCCACCATGCCAATGAG
AACATCTTCTACTGCCCCATTGCCATCATGTCAGCTCTAGCCATG
GTATACCTGGGTGCAAAAGACAGCACCAGGACACAAATAAATAAG
GTTGTTCGCTTTGATAAACTTCCAGGATTCGGAGACAGTATTGAA
GCTCAGTGTGGCACATCTGTAAACGTTCACTCTTCACTTAGAGAC
ATCCTCAACCAAATCACCAAACCAAATGATGTTTATTCGTTCAGC
CTTGCCAGTAGACTTTATGCTGAAGAGAGATACCCAATCCTGCCA
GAATACTTGCAGTGTGTGAAGGAACTGTATAGAGGAGGCTTGGAA
CCTATCAACTTTCAAACAGCTGCAGATCAAGCCAGAGAGCTCATC
AATTCCTGGGTAGAAAGTCAGACAAATGGAATTATCAGAAATGTC
CTTCAGCCAAGCTCCGTGGATTCTCAAACTGCAATGGTTCTGGTT
AATGCCATTGTCTTCAAAGGACTGTGGGAGAAAGCATTTAAGGAT
GAAGACACACAAGCAATGCCTTTCAGAGTGACTGAGCAAGAAAGC
AAACCTGTGCAGATGATGTACCAGATTGGTTTATTTAGAGTGGCA
TCAATGGCTTCTGAGAAAATGAAGATCCTGGAGCTTCCATTTGCC
AGTGGGACAATGAGCATGTTGGTGCTGTTGCCTGATGAAGTCTCA
GGCCTTGAGCAGCTTGAGAGTATAATCAACTTTGAAAAACTGACT
GAATGGACCAGTTCTAATGTTATGGAAGAGAGGAAGATCAAAGTG
TACTTACCTCGCATGAAGATGGAGGAAAAATACAACCTCACATCT
GTCTTAATGGCTATGGGCATTACTGACGTGTTTAGCTCTTCAGCC
AATCTGTCTGGCATCTCCTCAGCAGAGAGCCTGAAGATATCTCAA
GCTGTCCATGCAGCACATGCAGAAATCAATGAAGCAGGCAGAGAG
GTGGTAGGGTCAGCAGAGGCTGGAGTGGATGCTGCAAGCGTCTCT
GAAGAATTTAGGGCTGACCATCCATTCCTCTTCTGTATCAAGCAC
ATCGCAACCAACGCCGTTCTCTTCTTTGGCAGATGTGTTTCCCCT
GGCGGCGGCTGAGTCGAC

A DNA sequence coding a truncated chicken ovalbuimin lacking 153 nucleotides at the 5′ end (Seq 7) was used to replace the GFP coding sequence in the Mrc1.GFP.miRT LV using Agel and Sall to originate the Mrc1.OVA.miRT.

Seq 7. Truncated OVA
(SEQ ID NO: 52)
ACCGGTCCACAAAGACAGCACCATGACACAAATAAATAAGGTTGT
TCGCTTTGATAAACTTCCAGGATTCGGAGACAGTATTGAAGCTCA
GTGTGGCACATCTGTAAACGTTCACTCTTCACTTAGAGACATCCT
CAACCAAATCACCAAACCAAATGATGTTTATTCGTTCAGCCTTGC
CAGTAGACTTTATGCTGAAGAGAGATACCCAATCCTGCCAGAATA
CTTGCAGTGTGTGAAGGAACTGTATAGAGGAGGCTTGGAACCTAT
CAACTTTCAAACAGCTGCAGATCAAGCCAGAGAGCTCATCAATTC
CTGGGTAGAAAGTCAGACAAATGGAATTATCAGAAATGTCCTTCA
GCCAAGCTCCGTGGATTCTCAAACTGCAATGGTTCTGGTTAATGC
CATTGTCTTCAAAGGACTGTGGGAGAAAGCATTTAAGGATGAAGA
CACACAAGCAATGCCTTTCAGAGTGACTGAGCAAGAAAGCAAACC
TGTGCAGATGATGTACCAGATTGGTTTATTTAGAGTGGCATCAAT
GGCTTCTGAGAAAATGAAGATCCTGGAGCTTCCATTTGCCAGTGG
GACAATGAGCATGTTGGTGCTGTTGCCTGATGAAGTCTCAGGCCT
TGAGCAGCTTGAGAGTATAATCAACTTTGAAAAACTGACTGAATG
GACCAGTTCTAATGTTATGGAAGAGAGGAAGATCAAAGTGTACTT
ACCTCGCATGAAGATGGAGGAAAAATACAACCTCACATCTGTCTT
AATGGCTATGGGCATTACTGACGTGTTTAGCTCTTCAGCCAATCT
GTCTGGCATCTCCTCAGCAGAGAGCCTGAAGATATCTCAAGCTGT
CCATGCAGCACATGCAGAAATCAATGAAGCAGGCAGAGAGGTGGT
AGGGTCAGCAGAGGCTGGAGTGGATGCTGCAAGCGTCTCTGAAGA
ATTTAGGGCTGACCATCCATTCCTCTTCTGTATCAAGCACATCGC
AACCAACGCCGTTCTCTTCTTTGGCAGATGTGTTTCCCCTTAAGT
CGAC
A DNA sequence encoding IL10 used in this
study was:
(SEQ ID NO: 39)
ATGCCAGGCTCCGCCCTGCTGTGCTGTCTGCTGCTGCTGACCGGC
ATGAGGATCAGCAGAGGACAGTACTCCCGGGAGGACAACAATTGC
ACCCACTTCCCTGTGGGACAGTCCCACATGCTGCTGGAGCTGCGC
ACAGCTTTTTCTCAGGTGAAGACCTTCTTTCAGACAAAGGACCAG
CTGGATAACATCCTGCTGACCGACAGCCTGATGCAGGATTTCAAG
GGCTACCTGGGATGTCAGGCCCTGTCCGAGATGATCCAGTTTTAT
CTGGTGGAGGTGATGCCTCAGGCTGAGAAGCACGGCCCCGAGATC
AAGGAGCACCTGAATTCTCTGGGAGAGAAGCTGAAGACACTGCGG
ATGCGCCTGAGGAGATGCCACAGGTTCCTGCCTTGTGAGAACAAG
TCTAAGGCCGTGGAGCAGGTGAAGAGCGACTTTAATAAGCTGCAG
GATCAGGGCGTGTACAAGGCCATGAACGAGTTCGATATCTTTATC
AATTGCATCGAGGCTTATATGATGATCAAGATGAAGAGCTGA
A DNA sequence encoding single chain IL12
used in this study was:
(SEQ ID NO: 40)
atgtgtcctcagaagctaaccatctcctggtttgccatcgttttg
ctggtgtctccactcatggccatcgccgggcaattgatgtgggag
ctggagaaagacgtttatgttgtagaggtggactggactcccgat
gcccctggagaaacagtgaacctcacctgtgacacgcctgaagaa
gatgacatcacctggacctcagaccagagacatggagtcataggc
tctggaaagaccctgaccatcactgtcaaagagtttctagatgct
ggccagtacacctgccacaaaggaggcgagactctgagccactca
catctgctgctccacaagaaggaaaatggaatttggtccactgaa
attttaaaaaatttcaaaaacaagactttcctgaagtgtgaagca
ccaaattactccggacggttcacgtgctcatggctggtgcaaaga
aacatggacttgaagttcaacatcaagagcagtagcagttcccct
gactctcgggcagtgacatgtggaatggcgtctctgtctgcagag
aaggtcacactggaccaaagggactatgagaagtattcagtgtcc
tgccaggaggatgtcacctgcccaactgccgaggagaccctgccc
attgaactggcgttggaagcacggcagcagaataaatatgagaac
tacagcaccagcttcttcatcagggacatcatcaaaccagacccg
cccaagaacttgcagatgaagcctttgaagaactcacaggtggag
gtcagctgggagtaccctgactcctggagcactccccattcctac
ttctccctcaagttctttgttcgaatccagcgcaagaaagaaaag
atgaaggagacagaggaggggtgtaaccagaaaggtgcgttcctc
gtagagaagacatctaccgaagtccaatgcaaaggcgggaatgtc
tgcgtgcaagctcaggatcgctattacaattcctcatgcagcaag
tgggcatgtgttccctgcagggtccgatcccggcgcgccggcggc
ggcggcagcggcggcggcggcagcggcggcggcggcagccgtacg
agggtcattccagtctctggacctgccaggtgtcttagccagtcc
cgaaacctgctgaagaccacagatgacatggtgaagacggccaga
gaaaaactgaaacattattcctgcactgctgaagacatcgaccat
gaagacatcacacgggaccaaaccagcacattgaagacctgttta
ccactggaactacacaagaacgagagttgcctggctactagagag
acttcttccacaacaagagggagctgcctgcccccacagaagacg
tctttgatgatgaccctgtgccttggtagcatctatgaggacttg
aagatgtaccagacagagttccaggccatcaacgcagcacttcag
aatcacaaccatcagcagatcattctagacaagggcatgctggtg
gccatcgacgagctgatgcagtctctgaatcataatggcgagact
ctgcgccagaaacctcctgtgggagaagcagacccttacagagtg
aaaatgaagctctgcatcctgcttcacgccttcagcacccgcgtc
gtgaccatcaacagggtgatgggctatctgagctccgccacgcgt
gctagctga

### LV Production

Vesicular stomatitis virus (VSV)-pseudotyped, third-generation LVs were produced by transient five-plasmid co-transfection into 293T cells, as described previously (Soldi, M., et al., Molecular Therapy: Methods & Clinical Development, 2020. in press). Briefly, 9 million 293T cells were seeded in 15 cm dishes 24 h before transient transfection in 20 ml of cell culture medium. For each dish, a plasmid mix was prepared containing (i) the envelope plasmid (VSV-G, 9 μg), (ii) the packaging pMDLg/pRRE plasmid (12.5 μg), (iii) the REV plasmid (6.25 μg), (iv) the pADVANTAGE plasmid (15 μg), and (v) the transfer lentiviral plasmid (32 μg). Transient transfection was performed as described previously (Soldi, M., et al., Molecular Therapy: Methods & Clinical Development, 2020. in press). After 30 h, the cell supernatant was collected, filtered (0.22 μm), and concentrated by ultracentrifugation using a Beckman ultracentrifuge equipped with a SW32Ti rotor, at 82′600 RCF for 2 h at 20° C. LV particles were collected in PBS and stored at −80° C.

The purified lentiviral vector (LV) was produced following the medium-scale process development laboratory (PDL) protocol (Soldi, M., et al., Molecular Therapy: Methods & Clinical Development, 2020. in press). Briefly, LV was produced by calcium phosphate-mediated transient transfection of adherent HEK293T cells, in Cell Factory 10-tray stacks (CF10), using the standard 3rd generation system comprised of vector transfer plasmid and plasmids encoding for the HIV gag-pol gene, the HIV rev gene and the vesicular stomatitis virus envelope G glycoprotein as described above. In addition, the pAdVAntage was added to the pool of plasmids. After 14-16 h post-transfection the medium was replaced and supplemented with sodium butyrate at 1 mM final concentration. The LV-containing supernatant (˜6 liters) was harvested 30 h after medium change, filtered and clarified through 5 μm and 0.8-0.45 μm filters, respectively, to remove cell debris and large aggregates and then loaded to anion exchange chromatography overnight at 5-10° C. After washing with a low salt concentration buffer, the vector particles bound to the column were eluted with a linear salt gradient from 0 to 1 M NaCl. To reduce the high salt concentration, a one-to-one dilution of the LV sample with PBS was performed, immediately after elution. The diluted LV was subsequently concentrated by tangential flow filtration (TFF) system. Benzonase treatment was performed twice, at 16 U/ml and 50 U/ml respectively, in presence of 2 mM MgCl2 for 4 h, at 4° C., before and after the capturing step to digest contaminant DNA. Finally, GF chromatography was employed as a polishing step to allow buffer exchange. LV was eluted in a volume of ˜15 ml PBS, achieving a final ˜500-fold volume concentration from the starting cell medium harvest. The purified vector stock was finally filtered with 0.2 μm membranes in order to eliminate the risk of microbial contamination in the final product and stored at −80° C. The vector batch was then analyzed for host (HEK293T) cell DNA and proteins, residual plasmid content, endotoxin levels and aggregates to determine product purity and safety.

LV stocks produced by the PDL or the ultracentrifugation were titred on 293T cells. The titers of LVs were calculated by measuring the copy number of vector integrated per genome by quantitative digital droplet PCR, as described previously (Soldi, M., et al., Molecular Therapy: Methods & Clinical Development, 2020. in press). We obtained titers ranging from 10⁹ to 10¹⁰ transducing units (TU)/ml after LV ultracentrifugation or purification.

### BMDM Generation and Transduction

BM cells were obtained from 6-week-old C57BL6 mice from femurs and tibias. BM cells were then incubated in RPMI medium supplemented with MCSF (100 ng) for 7 days to obtain adherent BMDMs. At day 7 BMDMs were transduced at MOI 10, after 24h medium was replaced. Transduced BMDMs were polarized by adding IL4 (20 ng/ml, Peprotech) for 24-72 h, or LPS (100 ng/ml, Sigma)+IFN-g (200 U/ml, Peprotech) for 24-48 h in the RPMI medium supplemented with M-CSF (100 ng/ml). BMDMs for flow cytometry analysis were cultured on Petri dishes (non-tissue culture treated, bacterial grade).

### MC38 Culture and Transduction

MC38 cells were cultured in IMDM medium supplemented with 10% FBS in 15 cm dishes (FALCON) and split 3 times per week in a ratio of 1/10 keeping them at a maximum confluency of 80%.

In order to produce MC38 cells expressing OVA, we transduced MC38 cells as previously described (Squadrito, M. L., et al., Nat Methods, 2018. 15(3): p. 183-186), with an LV expressing OVA from a constitutive PGK promoter (PGK.OVA LV, described above). Briefly, MC38 cancer cells were transduced with LV doses ranging from multiplicity of infection (MOI) 1 to 20. VCN was analysed by using digital droplet PCR and transduced MC38 cells with a VCN similar to 3 were used in experiments. Transduced cells were propagated for several days, and stored in liquid nitrogen.

To obtain mCherry-positive MC38, we transduced MC38 cells with an LV expressing an mCherry fluorescent protein from a constitutive PGK promoter, described previously (Squadrito, M. L., et al., Nat Methods, 2018. 15(3): p. 183-186). As above, MC38 cancer cells were transduced with LV doses ranging from MOI 1 to 20. The lowest MOI leading to 100% mCherry expression in MC38 cells was used in further experiments.

### Organoids Culture

Organoids were kept in culture as previously described (Sakai, E., et al., Cancer Res, 2018. 78(5): p. 1334-1346; and Nakayama, M., et al., Nat Commun, 2020. 11(1): p. 2333). In brief, organoids were cultured in in 30 μL droplets of growth factor reduced Matrigel (BD) in a 48 well plate (Costar) covered with 300 μL Advanced F12/DMEM medium (Thermo Fisher Scientific) supplemented with Hepes (10 mM; Thermo Fisher Scientific), GlutMAX (2 mM; Thermo Fisher Scientific), N2-supplement (1×; Gibco), B27-supplement (1×/Thermo Fisher Scientific), recombinant mouse EGF (50 ng/mL; Invitrogen), N-Acetyl-Cysteine (1 mM; Sigma-Aldrich) and 1% Pen/Strep. For spitting, medium was removed, organoids were recovered using 500 μL Cell Recovery solution (Corning), washed with ice cold PBS (Corning) and suspended in fresh Matrigel in a splitting ratio of 1:5.

### In Vivo Studies

C57BL6 and Swiss Nude mice were purchased from Charles River and maintained in specific-pathogen-free (SPF) conditions. The procedures involving animals were designed and performed with the approval of the Animal Care and Use Committee of the San Raffaele Hospital (IACUC #1007 and #1098) and communicated to the Ministry of Health and local authorities according to Italian law.

LV doses in the range of 3×10\hat{ }7-3×10\hat{ }8 were delivered to mice by tail vein injection in total 250 μl of PBS.

MC38 cells were delivered to mice through intrahepatic injections to originate single experimental liver metastases. Briefly, MC38 cells were detached by using trypsin and resuspended in PBS. 1×10\hat{ }5-5×10\hat{ }5 MC38 cells in 5 μl of PBS were delivered to either C57BL6 or Swiss Nude mice.

To originate multiple spontaneously seeding liver metastases, CRC organoids were mechanically dissociated, and 3×10\hat{ }5 organoid cells were injected with Matrigel into the spleen of C57BL/6.

Mice were subjected to abdominal MRI to measure eventual liver metastases. MRI studies were performed by using a 7-Tesla MR scanner (Bruker, BioSpec 70/30 USR, Paravision 5.1, Germany). MRI was performed in mice previously treated with an intravenous injection of gadoxetic acid (Gd-EOB-DTPA; Primovist, Bayer Schering Pharma).

### Tumor and Liver Dissociation

For preparation of liver metastases, liver and spleen for flow cytometry analysis, mice were euthanized by cervical dislocation. The whole liver was perfused by injection of 10 ml PBS supplemented with 5 mM of EDTA and 10 ml IMDM (Corning) containing collagenase type IV (Sigma Aldrich) at a concentration of 35 μg/ml. A small section of the required tissue was taken and manually cut into small pieces followed by incubation in IMDM containing collagenase type IV (Sigma Aldrich) at a concentration of 35 μg/ml and 1 mg/ml Dispase II (Roche) at 37° C. for 15 minutes. Cell suspension were then filtered using a 40 μm cell strainer (Falcon) and washed with 30 ml MACS Buffer (Miltenyi Biotec). Flow cytometry analysis was performed as described below.

### Blood Processing

Blood was taken from the tail vein of the mice and collected in Microvette® 500 K3E tubes (Sarstedt). For flow cytometry analysis, an additional red blood lysis step was performed after antibody staining using the Red Blood Cell Lysing Buffer Hybri-Max™ (Sigma). Hemocytometer analysis was performed on full blood using the ProCyte DX™ (IDEXX). For collection of plasma, blood was centrifuged for 10 minutes at 845×G and supernatant was collected.

### IFNα Quantification

The IFNα level in the plasma was quantified by ELISA assay using the IFN□ high sensitivity kit from PBL Assay Science (catalogue number: 42115-1) following manufacturer's instructions. Samples were measured in technical duplicates in the following dilutions: 1/10 or 1/50.

### Flow Cytometry

For immunophenotypic analyses (performed on FACSCanto II or Symphony; BD PharMingen), we used the antibodies listed in the table below. Single stained and Fluorescence Minus One stained cells were used as controls. Either 7-AAD Viability Staining Solution (BioLegend) or the LIVE/DEAD™ Fixable Blue Dead Cell Stain Kit (Invitrogen) staining were performed to exclude dead cells from the analysis.

### List of Antibodies for Flow Cytometry Analysis

| Target/compound | Clone | Supplier |
|---|---|---|
| CD11b | M1/70 | Biolegend |
| CD11c | N418 | Biolegend |
| MRC1 | C068C2 | Biolegend |
| LNGFR | C40-1457 | BD Pharmagen |
| PD1 | 29F.1A12 | BioLegend |
| CD31 | MEC13.3 | Biolegend |
| CD4 | RM4-5 | BD Horizon |
| CD45 | 30-F11 | Biolegend |
| B220 | RA3-6B2 | BD Pharmagen |
| B220 | RA3-6B2 | Biolegend |
| CD86 | GL-1 | Biolegend |
| CD8a | 53-6.7 | BD Pharmagen |
| F4/80 | BM8 | Biolegend |
| H-2Kb - SIINFEKL | F093 | Prolmmune |
| LY6C | HK1.4 | eBioscience |
| Ly6G | 1A8 | Biolegend |
| LY6G | 1A8 | Biosciences |

### Immunofluorescence Analysis

Liver metastases were cut into 10-20 μm cryostatic sections for immunofluorescence staining and confocal microscopy. Briefly, tumors were fixed for 2 hr in 4% paraformaldehyde, equilibrated for 12 hr in PBS containing 10% sucrose, 12 hr in PBS/20% sucrose, and eventually 12 hr in PBS/30% sucrose. The samples were then embedded in Killik OCT embedding medium (Bio-optica) on dry ice and then stored at −80° C. Cryostatic sections were laid on slides for immediate staining. Sections were then blocked with 5% fetal bovine serum in PBS containing 1% bovine serum albumin (BSA) and 0.1% Triton X-100 (PBS-T).

The following primary antibodies were used: chicken anti-GFP (AB13970, Abcam) at 1:500 working dilution; rabbit anti-mCherry (AB167453, Abcam) at 1:200 working dilution; rabbit anti-CD31 (PA5-16301, Invitrogen) at 1:200 working dilution; rat anti-F4/80 (AB6640, Abcam) at 1:200 working dilution. Goat antibodies were used for secondary staining, at 1:500 working dilution: AF488 anti-Chicken (A-11039, Invitrogen), AF647 anti-Rat (A-21247, Invitrogen) and AF546 anti-Rabbit (A-11010, Invitrogen). Counterstaining of the nuclei was done using Hoechst, diluted 1:2000 in PBS. Immunofluorescence pictures were acquired with a Leica TCS SP8 Laser Scanning Confocal, with HC PL FLUOTAR 10× (NA 0.3) Dry and HC PL APO CS 20X (NA 0.7) Dry objectives. Images were analyzed using Fiji ImageJ software.

RNA Extraction and cDNA Generation

Tissues collected from mice were stored at −80° C. RNA was extracted from 30 mg of tissue using the RNeasy® Mini Kit (Qiagen) and quantified by UV/Vis using a Nanodrop 8000 (Thermo Scientific). RNA was immediately processed for cDNA generation using the SuperScript™ IV VILO (Invitrogen) and cDNA was stored at −20° C.

### DNA Extraction

Tissues collected from mice were stored at −80° C. For DNA extraction a piece of 25 mg was used and processed using the DNeasy® Blood & Tissue Kit (Qiagen). Extracted DNA was quantified by UV/Vis using a Nanodrop 8000 (Thermo Scientific) and stored at 4° C.

### Gene Expression and VCN Determination

For gene expression analysis. Digital droplet PCR was employed using the Automated Droplet Generator (Bio-Rad) for droplet generation, the T100™ Thermal Cycler (Bio-Rad) for PCR amplification and the QX200™ Droplet Reader (Bio-Rad) for acquisition. TaqMan assays for Irf7, Ifit1 or Oas1a using a probe labeled with FAM were combined with the TaqMan assay for the normalizer gene Hprt using a probe labelled with HEX. For VCN determination the TaqMan assays targeting HIV (FAM) and Sema3a (HEX) were combined. Analysis was performed using the QuantaSoft™ Analysis Pro software (Bio-Rad). The VCN was calculated by using the following equation:

$$VCN = \frac{concentration of HIV gene}{concentration of Sema gene} \times 2$$

### List of TaqMan Assays Used for Gene Expression and VCN Determination

| | | | Forward | Primer | |
|---|---|---|---|---|---|
| Target | Supplier | Assay number | primer | reverse | Sequence probe |
| Irf7 | ThermoFisher | Mm00516788_m1 | | | |
| Ifit1 | ThermoFisher | Mm00515153_m1 | | | |
| Oas1a | ThermoFisher | Mm00836412 m1 | | | |
| Hprt | | Mm03024075_m1 | | | |
| HIV | ThermoFisher | Custom design | TACTGAC | TCTCGACG | FAM- |
| | | | GCTCTCG | CAGGACTC | ATCTCTCTCCT |
| | | | CACC | G (SEQ ID | TCTAGCCTC |
| | | | (SEQ ID | NO: 54) | (SEQ ID NO: 55) |
| | | | NO: 53) | | |
| Sema | Metabion | Custom design | ACCGATT | TCCATATTA | HEX- |
| | | | CCAGATG | ATGCAGTG | AGAGGCCTGT |
| | | | ATTGGC | CTTGC | CCTGCAGCTCA |
| | | | (SEQ ID | (SEQ ID | TGGBHQ-1 |
| | | | NO: 56) | NO: 57) | (SEQ ID NO: 58) |

All publications mentioned in the above specification are herein incorporated by reference. Various modifications and variations of the disclosed vectors, cells, compositions, methods and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A vector for liver and/or splenic phagocyte-specific expression, wherein the vector comprises a transgene operably linked to one or more expression control sequence.

2. The vector of claim 1, wherein the one or more expression control sequence comprises:
(a) a phagocyte-specific promoter and/or enhancer; and/or
(b) one or more miRNA target sequence.

3. The vector of claim 2, wherein the phagocyte is a liver and/or splenic phagocyte.

4. The vector of claim 1, wherein the phagocyte is a macrophage.

5. The vector of claim 1, wherein the phagocyte is a Kupffer cell.

6. The vector of claim 2, wherein the phagocyte-specific promoter and/or enhancer is selected from the group consisting of: a MRC1 promoter and/or enhancer; an ITGAM promoter and/or enhancer; a CD86 promoter and/or enhancer; a CD274 promoter and/or enhancer; a CD163 promoter and/or enhancer; a LYVE1 promoter and/or enhancer; a STAB1 promoter and/or enhancer; a ITGAX promoter and/or enhancer; a SIRPA promoter and/or enhancer; a TIE2 promoter and/or enhancer; a CHIL3 promoter and/or enhancer; a CD68 promoter and/or enhancer; a CSF1R promoter and/or enhancer; a VCAM1 promoter and/or enhancer; a PTGS1 promoter and/or enhancer; and a C1QA promoter and/or enhancer; a fragment thereof, and a combination thereof.

7. The vector of claim 2, wherein the phagocyte-specific promoter and/or enhancer is a MRC1 promoter and/or enhancer or a fragment thereof.

8. The vector of claim 2, wherein the one or more miRNA target sequence: (a) suppresses expression in cells other than liver phagocytes; and/or (b) suppresses transgene expression in hepatocytes and/or liver sinusoidal endothelial cells and/or splenic phagocytes.

9. The vector of claim 2, wherein the one or more miRNA target sequence comprises: (a) one or more miR-126 target sequence; and/or (b) one or more miR-122 target sequence.

10-11. (canceled)

12. The vector of claim 1, wherein the transgene encodes a cytokine or a tumour antigen.

13. The vector of claim 1, wherein the transgene encodes interferon-alpha.

14-16. (canceled)

17. The vector of claim 1, wherein the vector is a viral vector.

18. The vector of claim 1, wherein the vector is a lentiviral vector.

19-21. (canceled)

22. The vector of claim 1, wherein the transgene is substantially not expressed in cells other than the phagocytes, when transduced by the vector.

23. The vector of claim 1, wherein the transgene is substantially only expressed in liver cells and/or splenic cells.

24. A cell comprising the vector of claim 1.

25. (canceled)

26. A cancer vaccine comprising the vector of claim 1.

27. A method of treatment comprising administering the vector of claim 1 to a subject in need thereof.

28. (canceled)

29. A method of treating or preventing cancer comprising administering the vector of claim 1 to a subject in need thereof.

30-31. (canceled)

32. The vector of claim 1, wherein the phagocyte is an M2-like macrophage and/or MRC1+ macrophage; dendritic cell; or liver sinusoidal endothelial cell.


SEQUENCE LISTING (58 sequences)

SEQ ID NO: 1 - DNA, 1895 bp, Artificial Sequence
  Exemplary human MRC1 promoter
ccctgaatgtgattatatacataattcaattaaatgtatttgcttctgaaatatatataaatgtaaattaggcagtcacttttgtatatgatttatttatatttgaaagccacaaatgacccatttaaactattattttcataagccagtgaaacaatgtctgagaaacatttttgttttgtctgttctgttctataaccatcattttttttttcagtcatgtacagccttagtgacaaagaaactttggtcctctgtcctacattttcactatctttttccctccggtcaggataatctcaaatttacatgttaaaaacaatcagtaagagaactacatcacatttctaataggatggaaacttttcaactttatcacaaagacaacgaatgtggaggctttccgtttgaagataaaactattcatttaaaaaattttaaaaattacaatgtttccagtagcttctttttgaattactaacatattccacactctagtaacggtttggccagctaatcgttagtttctgctttaaaatgttctaaattcctgttctacttttgaaaaatgacaacataaatgtttggagggttattttctgcttaatgaaagatctagaaacatattttattctaagaaagaattccacttgcctttaaataaagatataccttttgaccaaacaatcagattttctttttcttttttttcttttcttttttttttttgagatggagtttcgcgtctgtcgcccaggctggagtgtagtggtgcgatcctgactcactgtaacttccacttcccaggttcaaacgattctgttgcctcagcctcctgagtagctgggcttacaggtgtgcatgatcacacccggctaacttttgtatttttagtagagacgggtttttgccatgttgaccaggctggtttcaaactcctgacctcgggtgatctgactgcctcggcctcccaaactgctgggattgcaggcgtgagccattgtgcctggccagattttctttttctagcaaggggacccacttaaacttgaagaggaccgggatggttgaggctgggcagcaaggctttactgcaaatcctttaccactgttttttctggctttctagagaacgttctagcaaaaggtttctagaactttctccttcctggcctgactgacattccctcttaggtgtagcctccttttcacttttcttctgcctggaggaaatgaagctccacggaactttctgttgaaactttccaagaaaaaaaagaaaggctctaagcactgaatgtggaaactgaaggggatgagcttcaactctgaagtgtttccagcgtaaaactgtcctttccagggcccgtgtggctgtcacttcagagtggaggttgtctgctgagggacccctgactcagctgcttcccaggggaagctccgtcttccggcacaggtaatggcctgcagcttgatctccacccagccccatctgagcaggccgggagctcccaggctgtttcacttctctccttcctgactcctcaccatcaccatcgccctctctcctccccaccccgccactcctctcccacacgtgtccctttctccccttcctctgcgtctgctcttctcagaagttagcttacgaagcaaagttgttactttgaattcctgtttttccagccaccctcatgtgacaggatgtctcctcagtagaggctttccctaaattcaggagccctttaaaagggagggcttcctctgtagttcttttcagctgggcagctctgggaacttggattaggtggagaggcagttggggggcctcgttgttttgcgtcttagttccgccctcctgtccatcaggagaaggaaaggataaaccctgggcc

SEQ ID NO: 2 - DNA, 1895 bp, Artificial Sequence
  Exemplary mouse MRC1 promoter
ctcgagccgagctctgaaatggatgcttcaaggatttgaagagacaccagaagtgaaaaacgtgctattttcccacagttcctggcaatacaaagattgttttaaggcctatggaaattcctcttcctccgttacctgaaattacagatttgtgttgacttgctcacccctcctaacctgataaaatcttccaataagataaaaatgatggagacaaatcctttgtgggatgttggacttcactttatatcacatccagcgtctcgttactgattctgattttattcctgtgcatgtaagacacgttgacataataaaaccatggatatacagatgcctgcaattcagttaactcttttttttcctcttcaaataagtcaaagcaaaccccaattaggcaaaacaatttgaatggcttgcatttaaaagaccaattaaaacattttttggtcagcaagcatgatgggacacacttataatcccagctctcagaaagtcaaaacagaggaaccaagaattcaaggccagcctgcgctacaaacgcaagactgtttcggtgttcctgtgataagtcagttacgcagtgattgaaaaggaaacgtttgcagcctctcaccagttgtgggagaattttctttgtcagttaagccttgatagaatgaaaaagaacggtgggtcccttctcagaatcttcctaatttaggctttttaaaaagaaaattcttgagagaaaccacagcttattgggaaatgagtgtgtacctgcctcagcgtggatgggtctgaacagcttttcacttgaaggtaaaccatctgtttacaacttctaagtcgccagtgtttccagagcttctttttgaaacgatgacatttcccacgctccagtttcaggtcttccctgactaaccacaaatatccatttctaaatattcttaattcttgttgaacgtctggaaaaaaaaaatcagtgtttaggtgggttgtgtggtgctttgtgaacgaccctgcaaaatcatgaagacgaaaccccactgtcatcgaatcaacaagcaacttttggactcaagccaggctttcttttgcaagagagagagagaggtcttccctttttcaaactctgaggactgtaatggttgaggcctggcagcgaaccgacaacaaagctattgccactatttcctctggctttctaaggaaagctgctagaactttctatccctgggcttcattgaggttgtcttaaaattaacttctgtcattttccttctagagacaggggcaaaactctacgtgaaccatacctttgatcctttccaaggagtgtgtgtgtgtgtgtgtgtgtgtgtgtgtgtgtgtgtgtgtgtgtgtgttggtgctcgggctctaagcctgagcaggaagagcttctgatgctttccagcgagtgtcctccctttctgactgtagaattgtgggtgagagcctccacagctgcctcctggagactttttcccacccagataatggcctccgtttggttactgcccagcacctgtggagagctcagcagggctgcctctccctgctgctcatggcctgggtcctcacttctccccacttcctgcgttttctcctctcctacacatgttcctctctccccttcctcctgtgccttagcttacgaagcaaagttgtaactttgaattcctgtttttctaaccgcccccatgtgacaggatatctctcaattggagggttttcctaaattcaggagtcctttaaaagggacagcttcctctgtcctccttttcagtcaggcagctcccagaccttggactgagcaaaggggcaacctggggacctggttgtattctttgcctttcccagtctccctcttctccctcattggaaccggt

SEQ ID NO: 3 - DNA, 22 bp, Artificial Sequence
  Exemplary miRT-126
cgcattattactcacggtacga

SEQ ID NO: 4 - DNA, 23 bp, Artificial Sequence
  Exemplary miRT-122
acaaacaccattgtcacactcca

SEQ ID NO: 5 - DNA, 139 bp, Artificial Sequence
  Exemplary miRT-122 4 x miRT
tctagataaacaaacaccattgtcacactccattcgaaacaaacaccattgtcacactccaacgcgtacaaacaccattgtcacactccaatgcatacaaacaccattgtcacactccacccgggtcgagctcggtacc

SEQ ID NO: 6 - DNA, 126 bp, Artificial Sequence
  Exemplary miRT-126 4 x miRT
ggtaccagcaaacgcattattactcacggtacgaccatcgcattattactcacggtacgaacttcgcattattactcacggtacgacgaacgcattattactcacggtacgacacgtgtcggtacc

SEQ ID NO: 7 - DNA, 237 bp, Artificial Sequence
  Exemplary miRT-122 and miR126 4 x miRT
ggtaccagcgctacaaacaccattgtcacactccaacatacaaacaccattgtcacactccagattacaaacaccattgtcacactccacagaacaaacaccattgtcacactccagtttaaacgcattattactcacggtacgaccatcgcattattactcacggtacgaacttcgcattattactcacggtacgacgaacgcattattactcacggtacgacacgtgtcggtacc

SEQ ID NO: 8 - protein, 189 aa, Artificial Sequence
  Exemplary human interferon-alpha
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 9 - protein, 187 aa, Artificial Sequence
  Exemplary human interferon-beta
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 10 - protein, 166 aa, Artificial Sequence
  Exemplary human interferon-gamma
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 11 - protein, 153 aa, Artificial Sequence
  Exemplary human Interleukin-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 219 aa, Artificial Sequence
  Exemplary human Interleukin-12 subunit alpha
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 13 - protein, 328 aa, Artificial Sequence
  Exemplary human Interleukin-12 subunit beta
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - protein, 233 aa, Artificial Sequence
  Exemplary human TNFa
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 15 - protein, 125 aa, Artificial Sequence
  Exemplary human CXCL9
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 16 - protein, 269 aa, Artificial Sequence
  Exemplary human IL-1B
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 17 - DNA, 444 bp, Artificial Sequence
  Mouse Mrc1 enhancer 1
acagaaccagcagtatagggaaggccgtggtgttgtgggactcacatgatattatttatgatatcttggaaattagagcaaagacaggttaggcattgtggtcagaggagctgggttatgacaccgaggaaacaagctgacccttgaattaaaacatattgacgccatagcaataagaggatggaaccacattgccctctgctgttggggaatcatggccgctgcccccattctgcagttaagagacccggtactgccctctgctggctggatgcacatgtttccacattctggattagtatccttttgaatttaaatttaaaaacagtctcctgctgcctgccagtgactcactgtggcctctttatgttgttagtagctttgttttactctggcagatagaaaatatgttacaggtcgccatcttggttccgggactcagca

SEQ ID NO: 18 - DNA, 503 bp, Artificial Sequence
  Human MRC1 enhancer 1
agccccaccatgttattgatggccaaacaatacgcatgctgacagccattatctgtggcctctgatgctattagccaaaccatgttattgatggtcaaacaatacgcatgctgacagccattatctgggactcagaaagttctgcatattcaagtcaggccagaggatccgagttctaatgttaagagaaaccaacacaccaacaagcaaataaacaaacctacccttgaaccaaaatatacatcaatacctccgttgcaaatggataaatggaactgcattgccctctgctgttggggaatcttggcaaccatttcaactctatggctggagatgacttactgctctgtttattttccatcctcctgcttagattattgctttcaaagtttccagaatagaagaagtcagtggtggccagttgtcctttaatggtctcttatctaccaatggctagtatcctttttgcattatcgtagctctactcttgtagatgttaaatt

SEQ ID NO: 19 - DNA, 748 bp, Artificial Sequence
  Mouse Mrc1 enhancer 2
acatgggaggcaaggcggaaggagcatgaggctgacctagcaggcaggaagcacagaaatcacattttgagctacatagaagaaggagagagagagagagagagagagagagagagagagagagagagagagagagagagagagagagagagagaaatcaggaagtgagactagtctataaaactgcaaagcctactcccactgacatacttcctttagcaatgcacagctgccacaaccctcccaaatcctgccaccaactggagaccaagggttacaataagtagacctaagggaggggtacttttcttttcaaccactgcagtggagcacacctctatgtccaacatgaaggaaatagaggctggaagaccagaaattcaaggtcacccaccagctcatcgccagttgcagatcagtttgagctacaggctatctgcctcaaatataaaactaaacagaaagtcaataaaaaggccacacttggggaagtggataatagggtcaaatattagtaaacacctcttcttccccattgttaaagcctgctccctccagttcctctgactttactgttacataacagatcttggacctgtgactgctgtgtttacaacatactcagtgacccctaacttctaatcatgaaacacatttacccggttccaggatgccatctctccacctacagctcaccatggaagcattttgcctcttagcaaaggtctttggtttctcgtgggtggca

SEQ ID NO: 20 - DNA, 1301 bp, Artificial Sequence
  Human MRC1 enhancer 2
tttattagattgtgaagactaaaagggctaaagtttgcttgagaagagagtatccgttttagcttctaaaatatacacttggggaaacagagagattaggatcagaaattagtgtgggagcaaaagcatccccgatttcttgcccccaaaatctcctccctccagtttttctggacacggctatattaatattagtctgtctctgctcctatgactgccatgttcaaaaaaccccttgtggcttctcatttcccatcaaataattaacaaaatacattttgcctgacattcaagattgccacctgctgaacatctgtactattcccggtcttgcattttgcggtcacaacaaaagaaaagtcagtcttggattccttgtttttcctcatccattctcaccctcacatcttttcccatgtcatttcttttgcctggaattctcacgattgtgtacagttgaaatgcaattctatttccatctgcatttctcttatttgcttattaccacttttattagtataatagctacttacacatatgtcctgcttccttgacaacactataaatacattaatttgttattatttatttacttattcattcattcccaagatatcttgtacccatgtgccaggctttgtgctaggtcctggggacatcaaagctgaccctgtcttgtccacatgcagcttaaaagtccaatgggaagaaacagatgtaaacaagaaaacaaacaaatgaataggtaccaattataataagtcctgcatggaaagcaaactgaaggccatgtggagatcgcgtcagagggggaaatagctgaaaacggggaatgtcttgtgaatatttatgacccctcttcccccaaggaggttcacacagagccttccaaggaagggtctcaccaaacatttgctgactgacatgttaacatgcaacaaaataaaaatacttagaaattcagtctcctgtttggaaaactagacagtcatggcaagaagacagtaaatgcagtggttcttctgtgtgaagtggcttattcatcaccttggaattctgttaaagtgatgatgcctgggctgagatcccgaagactctagatcagtaggtgtgacacagggcacaagaatctgccttttgtcaaactcctgggtgattctggggcagctggttgaaagaccatatctggcaaaacactgtgttagttcctgctgtatccaggtgctaacaccaggcagcagagcagagaggctgagagcaaagattctgcagccagactgcctaggtcccagctctgtgaa

SEQ ID NO: 21 - DNA, 1044 bp, Artificial Sequence
  Mouse Mrc1 enhancer 3
ggtcaactatatagtaatgaacacctatcaattattttccttaatatattagattttattcctcttaattcagcatcacttgcattctaatgaagatctctatgtccttccagccatgtactccttactgggcaatgcaaatggagccgtctgtgcatttccattcaagtttgaaaacaagtggtatgcagactgcacctctgccgggcgctcggacggatggctctggtgtggaaccaccactgactacgacaaagacaagctgtttggattttgtccattgcactgtaagtaactgaaaacagcacacctgggacattcagtatggtcacatgatggtagggtggactttatgtaccctctatctacctttctttgtttcttgtttcactttcacttctctctctctctctctttccttccccatctttctgtttgctaaggatcaaacccagccttgcacatgctaaacaaacactctactactgagctacatatcctgaccttattagttatttgctaagactttagggcaaattatactgaatatagcattatatatagtcagtgctggaggtaggtacatcgttctcccaaaccccaagtgttttagttttaaaaagccataggttaaagcaggaatttaaataacaccacaaacgagtttggtgggagtctgtgaaggctcttgcattttgcatcaccatgtgctggagctccttctagtacagatgatactgatggaggtttgtgaatctcaacgtaagaagggtggaattcagctgagttggcaatcaagggaagtgagtaatctatgcttcagtcctttgaaagcagaagtttggtgttactagagcatgcaagaccacataaagtaccagaacttgaattcttgaggttttatccattcgtaagaatctgtaagaaaatatgtggcagcttaggtggggctagggagggcagctgggagtcagagctagggctgagggaggagaaggttgaggtcttggcttaacttctgtatctctgaacatactttctgaa

SEQ ID NO: 22 - DNA, 2077 bp, Artificial Sequence
  Human MRC1 enhancer 3
cagcctcccgagtagctgggactacaggcgcccgccaccacgcctggctaattttttgtatttttagtagagacggggtttcactgtgttagccaggatggtctcgatctgctaacctcgtgatccgcccacctcgcctcccaaagtgttgggtttacaggcatgagccggcgcgcccgactatgacctctttatttttaccaactatcagttactcagcgaaaattatcctgtacacaatatagtatatacttaacacgggaggtaggtaggcatgctattattccctcatctctgaaatgccttacctttatgaaaccatagatcaaaaacagaattcaaaggaagccacaagcatgtggtttgatggagaaagacgtgaaggtcctgaattttgtgtcaatgtcatcaaaagtgttcttcacagtgaaaatgatagtcaggatactcttcttatgtttaaattaagaagggtgccatttagtttcacacagctagaaatcaatggaactcggtaatttatatgcttcaaggtttcttaaaaatgaaaattcaggttttattgcaaggatgcaaaataagcagtatcgcattatcttagtgggaaagcaccagaacagatgaattcttggtgttcttccacgcttaaaaatctgtaggcaaatagtgaagcacattttaggtggagtggaggagggcgtcatgttgaaggcagagctggggctgaggaaaaaggaaaaggaagtattcctcttagcttcacgttccccatcaccagacaccctcctcctgatgctggctccacccttcccaaacttcttacccccgacctctcacctgctactttagaccagatcagagtagctcttgtttcctggttatcacccagaactctttctcctgctgccctgcaaagggactgggcagagcaaagagcattcgatatggtctgggatgattgtgacaccacctgagtaacaatagaatcacgactatcacaactcaactttccagaccacaaatccacaagtaatcacactatttcaagcattattgtaaaacagaacaacttaaaaaatacctgaatttgacgaacaaaagccagaattctaagaattgtacttatttatctctctggatttataatccctaattatcacactaaaagtaaatttaatttctgagccccatatactattgtaattgtcttcagagtgcagtctctccaatccgaatgaatactcacaaaagcccataggctttctgttcatagcgacactgctgcctcggtcttaactgaggtagttctatttgtctctcttatgtcaatctttagaaagacatttgatttccattcaaggtttttagatgtcgaattttgtattcgaagtatttttgtctgaaacacattgagcaatttttttctaagataaagcaatacttggttttcaagtgattgaaagtgtctttctcctttacttaataggaatgatattttcttaatctgtttcatggactttcttaagggtatatatttcatgggtccaactatagcatcctccacatcctttgaaattgacaaaggagttagatgaatgtgtgatttcctgaatgaaatgtggaggacaagtggtaagttactaatcacaaagaaaactcacaatcttggaaatccttggatgtgtgttggagacgtatcttgagtttgttcagtggaataattttttagtcttattacttgtatttatgccttcactgtcaaattatatattttttcctgttaaatgtaaaataatcgtagaaaataaattgatttggtttcaatatgcattaaaattttaaatcacgttttgtacatttaatatctttcttaaagggctttatagtcttccagtctgtttcattttgtgttcttttcaaaagagtttttattgtatttatttatttatttatttttgagacagggtctcactctgtcatccagactggagtgcattagcatgatcttggctcaccacaacctctgcctcccaggctcaagtgattctcctgcctcagcctcctgagtagctgggat

SEQ ID NO: 23 - DNA, 321 bp, Artificial Sequence
  Mouse Mrc1 enhancer 4
aataaacgtctaggaacatttaccctaaagtactgccctctctatgtgaacaaacttaagcctgtgttctttcctttttgtgaacagacgcgaggcaatttttaatctataatgaagatcacaagcgctgcgtggacgctctaagtgccatctcagttcagacggcaacttgcaacccggaagctgaatcccagaaattccgctgggtgtcagattctcagatcatgagtgttgctttcaaattatgtttgggagtgccatcaaaaactgactgggcttccgtcaccctgtatgcctgtgattcgaaaagtgaatatcaga

SEQ ID NO: 24 - DNA, 311 bp, Artificial Sequence
  Human MRC1 enhancer 4
tggaagagttggaaacttttgacctaaaagatcgtccttgttacatgaatccacttagccatgcttgctttcttcttcttttcctgcttctttctttttaaacagacaccaggcaatttttaatctataatgaagatcacaagcgctgcgtggatgcagtgagtcccagtgccgtccaaaccgcagcttgcaaccaggatgccgaatcacagaaattccgatgggtgtccgaatctcagattatgagtgttgcatttaaattatgcctgggagtgccatcaaaaacggactgggttgctatcactctctat

SEQ ID NO: 25 - DNA, 426 bp, Artificial Sequence
  Mouse Mrc1 enhancer 5
tgtcaggttctctggagcaccctctcacctgttcagactaatttcctaagttcggcgggtcccggaccaagatggcgacccgctacatttcattcttacatgcaggggatgagcgcactgtttcaccactttgattgccttttttgagcatggtagatattcagtaagcaacccatggattgaattctactttatgtttaatgcaggacgaaaggcgggatgtgttgccatgaaaaccggagtggcaggtggcttatgggatgttttgagttgtgaagaaaaggcaaaatttgtgtgcaaacattgggcagaaggagtgactcgcccaccagagcccacaacaactcctgaacccaaatgtccagaaaactggggtaccaccagtaaaaccagcatgtgtttcaaagtaaggatcactcgccaaat

SEQ ID NO: 26 - DNA, 658 bp, Artificial Sequence
  Human MRC1 enhancer 5
catcctcattttattttatgtacttctttgttcgttaaagctggcattccttacagttctatgaggcaggtcttggtatttgcatttggagaggagaaagcaagttcagagcgtttgagtaacttacctaaaatctctagttgagacgtgtctcattttgaaatctgtgaaaaactttggtcctggaaaacctacgtagaccttagtgggaggaaaagaatcttaaaaaattaaaaaagaaagaaagcagagaaaaccagaaaggggaggaagggaagaaggaaggaaaaagggaaggaaggagggagggagagagaagcagtaaactatttttgccattatggtgaatttgataatataaaatattttatcattaaatgcctgtgtagggggcactttgccaaatgttagaaatataaagtgttacaaacccccctgcatctgagatcataattgggcatcagaaccctgatgctcggttctgagtgccttctgtgagcacggcaggccttcagcaggcacctgtcaagtgaattctacttcatatatttaatgcagggcgaaagccagggtgtgttgccatgagaaccgggattgcagggggcttatgggatgttttgaaatgtgatgaaaaggcaaaatttgtgtgcaagcactg

SEQ ID NO: 27 - DNA, 303 bp, Artificial Sequence
  Mouse Mrc1 enhancer 6
gagtgattgtgcatgaacttgtggagacctcaattgttcttgcaacttgtctcttctattactattgcaaaaggaatggctaagtctttcttgaaagaattcatatagttctctttcagagacctgcagcagttaccactttggggaactagagaaaagttatttttaagtttctctggaatgaaaggcacaattctataatttggccttattgcttaatccaccagttttaagttccttgtttgtaaaatatgaatgttagtaactcttcttctttaaaatctcgttatatcatcaagcttg

SEQ ID NO: 28 - DNA, 1001 bp, Artificial Sequence
  Human MRC1 enhancer 6
tttggccaagatcctaaatagatatagatgcgggacctggatgttgggtttgattatcctttacaggctctccatagtgacggtgggtatctttagagaaagctcaccatttttgcattttacctctactattcctcctctaggagaaatagtgtattttttccttttttggaagccttcattacaattctctttctaagacttggaatttccatgttgccaaagaggagaatagttactttatagtttctctggtacagcactcagaattctgtaacttggccttactgcttaacctgccggtgctgggatcctcatgtgtaaaatggaaatgttcatgactcttcttcctgagataaaattttgttcatttcatcaaacactcagtacattcttattcctccatgatgccttcttcactcgctaaccacttgatgtcagtttctgaacatctctatactccctggattaatgattctgttttatctataaactcaaataaaccagagcttggaaaagcgtatcagagttcaaattatgcagcatacgggattagcaacagccttaggcaagaattcaacctcaaaccctgtgaattattgtaacactaacccgtttgtccataatcctcaggtctctagggctgtactctctggcttagcagccacttaaccgcatgtggccactgagcacatgagctgtggctagaggaacaaatcatcttctgtggctgccccagggaactccccttcatttcactcaagttggttgttttcatgctttcaaatatgtttaaagttcatcatttcagtttttgaaggacagcattggctgatactattttcaatttcctaggtagcaaaattaaaataacccaccagagggctccaaagctgtactaagcttgctttctttttctttttcttctttttttttttgtaaccactggaagtgcacagaatctaaattgtattgagggaatagaatttttttaaatatgc

SEQ ID NO: 29 - DNA, 1312 bp, Artificial Sequence
  Mouse Mrc1 enhancer 7
atgagacgtctgtcctggtttgaactttgccaactgagccttattgccagcctgactgttactaggaatgggtcatgaaataaatgcttctgtcagaatagtttattcggattgaatgtgctctgcaacctctgctgacagccatcttcccgagtgtgtgcagcaaccagcccgaatgtgtcagcaatggctttcaggcacctgtgacacacgtatcacaagtaggatgttttgatgtttgcagggttatagcttcttcaggcaaaacttgcagggcatgaagaaagcagattcagcaaggaccttagcctgagcagctgactcgaatcgactgccaagtagcaaggaatctggcacgcgttctgagctccttggccagcctgaaccggctgaagctcaagcctcaagctcgcctctgcacccccgcacctccccccgccccccaccagtgcagacagttttcctgctttttgttgttgttcctttcttgttttatttaaaagccagattcctttcatgaagggcagcaaacatgtgagctctgcacatgcgcagcagtgaagaaattagctgaggaagttgaggctgtgtcagggcacctttcctgaagtggatccttggacatccaagccactgtgttcttttggcctgtcttcaacggagtacgttgtatggtgccaagcctcaggataccaaagaactgcttacaaaacacttgctccttcacaagaagcacagcagtttagccaagataacatcgctgccaagaactctcacataggctaagataaaaactgaaagccccagcacatgagagtgagttcttggtaggaaaaagcacagtaagttcttctcagccctcaccgtcaagatggctggcacgtgccacctactcagcagagatctggatgtctcaacagtatagttcacctttctgtgtggatgggcatctccctgtccactgagggcctaggaagaacaaaagcagcagcaggaggagtccactcttttgcttctagtcttcctgtttaagccgggagcttcccacctcactgtccttggaataggtttcacgccactggctggcttccctgctttgtgtgtcttagaacttagattgaattctaccacctgctcctctgggtcttcaacttgcagggaggtcttggaattatcagccttcacaatttggccagcctgaaccgctcaagctcaagcctcaagctcgcctctgcaaccccgcacctccccccccccgccccagtgcagacagttttcctgtttttgttgttgttcctttctgaattaattt

SEQ ID NO: 30 - DNA, 1387 bp, Artificial Sequence
  Human MRC1 enhancer 7
gaagaaaacacatccagttcttggaggaaaattgcaataaatattttgaagagagttccatctcttattctccctcaattttctgaaagtcagagtaacacttggctataaaagtgatagggaaactaagtgcctatcatataccaggcacagtggcatgcaatcaagtgggatttcatgtatttcccaagtgtgttttgctggctgccatgtaagaccctagtgttaattccaaaactcagaggtcctggctcttgaatgggtggggacaggaggtggatttaaagtttccagcagagaagaagtgtgggactgatcgtctgctggaaccagttctctgaatatgatggtttatctggcaaggtttgattccctcaaggaagttccaggctaaaagaggagctaagcttctacagtctctgagctttttgtgttactgatcttgagtcttattaaaaaaaaaaaaaaaaaaaaaaaaaccctgactttatctggccgtgccaggctcccttctgggcccttgctgccgtgtgtcagtacgctgtaactagagatggattatgtaagaaatgtttttgtcagaagaggctgttgcagtattttatgtgccctggtgcacagcacctctgctgtgagtcccctccccgtgtgagttgcagcctcgctggatacacctgctactggtctcaagcacctgtgatttattggtcatgaagcaggtactgggactcctgcttttattatctgcacggccatccctcagccagtatagatgcccaggctagacttgcagagcatgaagaaggaagcagaaccagtttgggaccttcgcctgagcagctgactcaactccactgccaagcagccaggaatctggcacagtttctgagatgtctcagccaactggtatttctgaagccatagttttcctctgagctccccctcaacatgggtatagtcattgtgcttgttttgcttcttccttgttcttgtttaaaagccagttttcttccttgaaaggcagtgaaatctctgggttttccatgtgggtagagaggagcaggcggaacaagcttagggaggccaggcggtaacaggtaaccatttcttgaagttgatcctcagagcatccaagccagtgggttcatttggatgatcttcagccaggcatagtccctggtgccaagcctcaggatatcaaagaactacttacaaaatatgtgttccttcataaggaaaagaatggtttagctcagagggcgtgcctgccaataaatctcacataggttaagacacaaactgaaaaaatgctcaaggaccatgagccacagtcactggagaagccacagtcattcattctccagcagttccccttaaccacctacca

SEQ ID NO: 31 - DNA, 1896 bp, Artificial Sequence
  Exemplary XhoI-Human.MRC1.promoter
ctcgagccctgaatgtgattatatacataattcaattaaatgtatttgcttctgaaatatatataaatgtaaattaggcagtcacttttgtatatgatttatttatatttgaaagccacaaatgacccatttaaactattattttcataagccagtgaaacaatgtctgagaaacatttttgttttgtctgttctgttctataaccatcattttttttttcagtcatgtacagccttagtgacaaagaaactttggtcctctgtcctacattttcactatctttttccctccggtcaggataatctcaaatttacatgttaaaaacaatcagtaagagaactacatcacatttctaataggatggaaacttttcaactttatcacaaagacaacgaatgtggaggctttccgtttgaagataaaactattcatttaaaaaattttaaaaattacaatgtttccagtagcttctttttgaattactaacatattccacactctagtaacggtttggccagctaatcgttagtttctgctttaaaatgttctaaattcctgttctacttttgaaaaatgacaacataaatgtttggagggttattttctgcttaatgaaagatctagaaacatattttattctaagaaagaattccacttgcctttaaataaagatataccttttgaccaaacaatcagattttctttttcttttttttcttttcttttttttttttgagatggagtttcgcgtctgtcgcccaggctggagtgtagtggtgcgatcctgactcactgtaacttccacttcccaggttcaaacgattctgttgcctcagcctcctgagtagctgggcttacaggtgtgcatgatcacacccggctaacttttgtatttttagtagagacgggtttttgccatgttgaccaggctggtttcaaactcctgacctcgggtgatctgactgcctcggcctcccaaactgctgggattgcaggcgtgagccattgtgcctggccagattttctttttctagcaaggggacccacttaaacttgaagaggaccgggatggttgaggctgggcagcaaggctttactgcaaatcctttaccactgttttttctggctttctagagaacgttctagcaaaaggtttctagaactttctccttcctggcctgactgacattccctcttaggtgtagcctccttttcacttttcttctgcctggaggaaatgaagctccacggaactttctgttgaaactttccaagaaaaaaaagaaaggctctaagcactgaatgtggaaactgaaggggatgagcttcaactctgaagtgtttccagcgtaaaactgtcctttccagggcccgtgtggctgtcacttcagagtggaggttgtctgctgagggacccctgactcagctgcttcccaggggaagctccgtcttccggcacaggtaatggcctgcagcttgatctccacccagccccatctgagcaggccgggagctcccaggctgtttcacttctctccttcctgactcctcaccatcaccatcgccctctctcctccccaccccgccactcctctcccacacgtgtccctttctccccttcctctgcgtctgctcttctcagaagttagcttacgaagcaaagttgttactttgaattcctgtttttccagccaccctcatgtgacaggatgtctcctcagtagaggctttccctaaattcaggagccctttaaaagggagggcttcctctgtagttcttttcagctgggcagctctgggaacttggattaggtggagaggcagttggggggcctcgttgttttgcgtcttagttccgccctcctgtccatcaggagaaggaaaggataaaccct

SEQ ID NO: 32 - DNA, 509 bp, Artificial Sequence
  Exemplary XhoI-Human.MRC1.enhancer
ctcgagagccccaccatgttattgatggccaaacaatacgcatgctgacagccattatctgtggcctctgatgctattagccaaaccatgttattgatggtcaaacaatacgcatgctgacagccattatctgggactcagaaagttctgcatattcaagtcaggccagaggatccgagttctaatgttaagagaaaccaacacaccaacaagcaaataaacaaacctacccttgaaccaaaatatacatcaatacctccgttgcaaatggataaatggaactgcattgccctctgctgttggggaatcttggcaaccatttcaactctatggctggagatgacttactgctctgtttattttccatcctcctgcttagattattgctttcaaagtttccagaatagaagaagtcagtggtggccagttgtcctttaatggtctcttatctaccaatggctagtatcctttttgcattatcgtagctctactcttgtagatgttaaatt

SEQ ID NO: 33 - DNA, 12 bp, Artificial Sequence
  Exemplary BamHI-KOZAC
ggatccgccacc

SEQ ID NO: 34 - DNA, 570 bp, Artificial Sequence
  Exemplary Human.IFNA
atggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 35 - DNA, 630 bp, Artificial Sequence
  Exemplary SalI-WPRE
gtcgacccgacagtttcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacc

SEQ ID NO: 36 - DNA, 225 bp, Artificial Sequence
  Exemplary AfeI-4xmiRT122-4xmiRT126-PmlI
agcgctacaaacaccattgtcacactccaacatacaaacaccattgtcacactccagattacaaacaccattgtcacactccacagaacaaacaccattgtcacactccagtttaaacgcattattactcacggtacgaccatcgcattattactcacggtacgaacttcgcattattactcacggtacgacgaacgcattattactcacggtacgacacgtgtc

SEQ ID NO: 37 - protein, 549 aa, Artificial Sequence
  Exemplary single chain human Interleukin-12
MCPQKLTISWFAIVLLVSPLMAIAGQLMWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAGGGGSGGGGSGGGGSRTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 38 - protein, 178 aa, Artificial Sequence
  Exemplary human Interleukin-10
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 39 - DNA, 537 bp, Artificial Sequence
  IL10
atgccaggctccgccctgctgtgctgtctgctgctgctgaccggcatgaggatcagcagaggacagtactcccgggaggacaacaattgcacccacttccctgtgggacagtcccacatgctgctggagctgcgcacagctttttctcaggtgaagaccttctttcagacaaaggaccagctggataacatcctgctgaccgacagcctgatgcaggatttcaagggctacctgggatgtcaggccctgtccgagatgatccagttttatctggtggaggtgatgcctcaggctgagaagcacggccccgagatcaaggagcacctgaattctctgggagagaagctgaagacactgcggatgcgcctgaggagatgccacaggttcctgccttgtgagaacaagtctaaggccgtggagcaggtgaagagcgactttaataagctgcaggatcagggcgtgtacaaggccatgaacgagttcgatatctttatcaattgcatcgaggcttatatgatgatcaagatgaagagctga

SEQ ID NO: 40 - DNA, 1674 bp, Artificial Sequence
  IL12
atgtgtcctcagaagctaaccatctcctggtttgccatcgttttgctggtgtctccactcatggccatcgccgggcaattgatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcccggcgcgccggcggcggcggcagcggcggcggcggcagcggcggcggcggcagccgtacgagggtcattccagtctctggacctgccaggtgtcttagccagtcccgaaacctgctgaagaccacagatgacatggtgaagacggccagagaaaaactgaaacattattcctgcactgctgaagacatcgaccatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgacgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccacgcgtgctagctga

SEQ ID NO: 41 - protein, 5 aa, Artificial Sequence
  linker
AGQLM

SEQ ID NO: 42 - protein, 20 aa, Artificial Sequence
  linker
RRAGGGGSGGGGSGGGGSRT

SEQ ID NO: 43 - protein, 4 aa, Artificial Sequence
  termination sequence
TRAS

SEQ ID NO: 44 - protein, 114 aa, Artificial Sequence
  Exemplary human Interleukin-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - protein, 157 aa, Artificial Sequence
  Exemplary human Interleukin-18
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 521 aa, Artificial Sequence
  Exemplary single chain human Interleukin-12
WELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAGGGGSGGGGSGGGGSRTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 47 - protein, 157 aa, Artificial Sequence
  Exemplary human Interleukin-18
YFGKLESKLSVIRNLNDQVLFIDQGNRPLFEDMTDSDCRDNAPRTIFIISAYGDSRARGKAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 48 - DNA, 30 bp, Artificial Sequence
  primer
aactcgagccgagctctgaaatggatgctt

SEQ ID NO: 49 - DNA, 29 bp, Artificial Sequence
  primer
aaccggttccaatgagggagaagagggag

SEQ ID NO: 50 - DNA, 625 bp, Artificial Sequence
  IFNa
accggtcagtcctccgacagactgagtcgcccgggggggatccaccggcatggctaggctctgtgctttcctgatggtcctggcggtgctgagctactggccaacctgctctctaggatgtgaccttcctcagactcataacctcaggaacaagagagccttgacactcctggtacaaatgaggagactctcccctctctcctgcctgaaggacaggaaggactttggattcccgcaggagaaggtggatgcccagcagatcaagaaggctcaagccatccctgtcctgagtgagctgacccagcagatcctgaacatcttcacatcaaaggactcatctgctgcatggaatacaaccctcctagactcattctgcaatgacctccaccagcagctcaatgacctgcaaggctgtctgatgcagcaggtgggggtgcaggaatttcccctgacccaggaagatgccctgctggctgtgaggaaatacttccacaggatcactgtgtacctgagagagaagaaacacagcccctgtgcctgggaggtggtcagagcagaagtctggagagccctgtcttcctctgccaatgtgctgggaagactgagagaagagaaatgagtcgac

SEQ ID NO: 51 - DNA, 1188 bp, Artificial Sequence
  OVA
ggatccgccaccatgggctccatcggcgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccctggcggcggctgagtcgac

SEQ ID NO: 52 - DNA, 1039 bp, Artificial Sequence
  Truncated OVA
accggtccacaaagacagcaccatgacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaagtcgac

SEQ ID NO: 53 - DNA, 18 bp, Artificial Sequence
  primer
tactgacgctctcgcacc

SEQ ID NO: 54 - DNA, 17 bp, Artificial Sequence
  primer
tctcgacgcaggactcg

SEQ ID NO: 55 - DNA, 20 bp, Artificial Sequence
  ATCTCTCTCCTTCTAGCCTC
atctctctccttctagcctc

SEQ ID NO: 56 - DNA, 20 bp, Artificial Sequence
  primer
accgattccagatgattggc

SEQ ID NO: 57 - DNA, 22 bp, Artificial Sequence
  primer
tccatattaatgcagtgcttgc

SEQ ID NO: 58 - DNA, 24 bp, Artificial Sequence
  probe
agaggcctgtcctgcagctcatgg